(19) United States
(12) Patent Application Publication
(10) Pub. No.: US 2014/0357662 A1
(43) Pub. Date: Dec. 4, 2014

(54) THIENO (2,3 - C) PYRAZOLES FOR USE AS POTASSIUM CHANNEL INHIBITORS

(71) Applicant: XENTION LIMITED, PAMPISFORD,CB (GB)

(72) Inventors: Derek Edward John, Pampisford (GB); Basil Hartzoulakis, Pampisford (GB); Simon D. Edwards, Pampisford (GB)

(21) Appl. No.: 14/358,721

(22) PCT Filed: Nov. 15, 2012
(86) PCT No.: PCT/GB2012/052841

§ 371 Date: May 15, 2014

(30) Foreign Application Priority Data
Nov. 15, 2011 (GB) 1119745.6
Aug. 28, 2012 (GB) 1215284.9

(51) Int. Cl. C07D 495/04

(52) U.S. Cl. CPC C07D 495/04
USPC 514/299; 546/199; 514/322; 548/360.5; 514/406; 546/112

(57) ABSTRACT

The present invention provides compounds of formula (I): wherein A, R¹, R², R³ _{I}X, and Z are defined herein, which are potassium channel inhibitors. The invention further provides pharmaceutical compositions comprising the compounds of formula (I) and their use in therapy, in particular in treatment of diseases or conditions that are mediated by Kir3.1 and/or Kᵢᵣ3.4 or any heteromultimers thereof, or that require inhibition of Kᵢᵣ3.1 and/or Kᵢᵣ3.4 or any heteromultimers thereof.

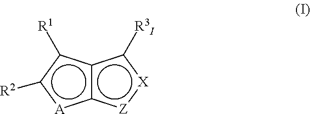

### TECHNICAL FIELD

The present invention relates to compounds of formula (I) which are potassium channel inhibitors. Pharmaceutical compositions comprising the compounds, their use in therapy and methods of treatment employing the compounds are also provided.

### BACKGROUND ART

Ion channels are proteins that span the lipid bilayer of the cell membrane and provide an aqueous pathway through which specific ions such as Na⁺, K⁺, Ca²⁺ and Cl⁻ can pass (Hille et al., 1999). Potassium channels represent the largest and most diverse sub-group of ion channels and they play a central role in regulating the membrane potential, cell volume, signal transduction controlling cellular excitability (Armstrong & Hille, 1998). Potassium channels have been categorized into gene families based on their amino acid sequence and their biophysical properties (for nomenclature see (Gutman et al., 2003) and http://www.iuphar-db.org/DATABASE/ReceptorFamiliesForward?type=IC).

Compounds which modulate potassium channels have multiple therapeutic applications in a number of areas/disorders including cardiovascular, neuronal, renal, metabolic, endocrine, auditory, pain, respiratory, immunological, inflammation, gastrointestinal, reproduction, cancer and cell proliferation, (for reviews see (Ehrlich, 2008; Wulff & Zhorov, 2008; Kobayashi & Ikeda, 2006; Mathie & Veale, 2007; Wulff et al., 2009; Camerino et al., 2008; Shieh et al., 2000; Ford et al., 2002; Geibel, 2005). More specifically potassium channels such as those formed by Kir3.x, Kv4.x, Kir2.x, Kir6.x, Kv11.x, Kv7.x, K_{Ca}, K_{2P}, and Kv1.x along with their ancillary subunit are involved in the repolarisation phase of the action potential in cardiac myocytes (Tamargo et al., 2004). These potassium channels subtypes have been associated with cardiovascular diseases and disorders including atrial arrhythmias, ventricular arrhythmias, cardiomyopathy, hypertrophy long QT syndrome, short QT syndrome, Brugada syndrome; and all of which can cause cardiac failure and fatality (Marban, 2002; Novelli et al., 2010; Tamargo et al., 2004).

Inwardly rectifying potassium channels are members of a large superfamily comprised of Kir1.x to Kir7.x. The Kir3.x subfamily are G-protein coupled inwardly rectifying potassium ion channels comprised of 4 mammalian subunit members Kir3.1 to Kir3.4. These subunits form homo- or hetero-tetrameric ion channels involved in potassium flux across the membrane. Kir3.x ion channels are expressed in the cardiovascular system (Kir3.1 and Kir3.4), central nervous system (Kir3.1, Kir3.2, Kir3.3>Kir3.4), gastrointestinal tract (Kir3.1 and Kir3.2) and have been implicated in a number of disease areas including cardiac arrhythmias, pain, Parkinson's disease, Down's Syndrome, epilepsy/seizure, addiction, depression and ataxia (Luscher & Slesinger, 2010; Tamargo et al., 2004) The human G-protein coupled inwardly-rectifying potassium channel subunits Kir3.1 and Kir3.4 are predominantly expressed in the supraventricular regions (including atria, nodal tissue, pulmonary sleeve) and conduction system of the heart and are believed to offer therapeutic opportunities for the management of atrial fibrillation for several different reasons (see review of (Ehrlich, 2008):

### (1) Kir3.1/3.4 Underlies IKACh:

There is evidence that a tetrameric assembly of Kir3.1 and/or Kir3.4 subunits underlies the cardiac acetylcholine/adenosine activated inwardly-rectifying potassium current (hereto referred to as IKACh) in the heart due to similar biophysical (Krapivinsky et al., 1995; Duprat et al., 1995; Corey & CLAPHAM, 1998; Corey et al., 1998) and pharmacological (Jin & Lu, 1998; Jin et al., 1999; Jin & Lu, 1999; Drici et al., 2000; Cha et al., 2006; Dobrev et al., 2005; Voigt et al., 2010b) properties (for review see (Hibino et al., 2010; Belardinelli et al., 1995)).

### (2) IKACh is Involved in AF:

The Kir3.1 subunit cannot form a functional homotetramer or cannot traffic to the membrane (Philipson et al., 1995; Hedin et al., 1996; Woodward et al., 1997) and as such genetic knockout of Kir3.4 gene in the mouse results in the lack of a functional IKACh in the atria (Wickman et al., 1998). This genetic ablation of IKACh results in resistance to atrial fibrillation (Kovoor et al., 2001). These data support the notion of an assembly of Kir3.1/3.4 and the importance of IKACh in the initiation and sustaining of AF. Furthermore, single nucleotide polymorphisms of Kir3.4 gene have been correlated with paroxysmal lone AF in a Chinese population (Zhang et al., 2009). However, no function has been ascribed to these polymorphisms.

### (3) IKACh is an Atrial-Specific Target:

High levels of Kir3.1 and Kir3.4 gene expression (Gaborit et al., 2007b) and large IKACh are found in both the left and right human atria (Dobrev et al., 2001; Dobrev et al., 2005; Voigt et al., 2010b; Wettwer et al., 2004; Bosch et al., 1999; Voigt et al., 2010a). This contrasts with the human ventricle, where mRNA (Gaborit et al., 2007b) and current expression are considerable smaller, and the number of cells expressing IKACh and the ACh sensitivity is small compared to the atria (Koumi & Wasserstrom, 1994; Koumi et al., 1994). In conjunction with a lower density of parasympathetic innervations (Kent et al., 1974), this argues against a functional role of I_{KACh} in human ventricles (Brodde & Michel, 1999; Belardinelli et al., 1995). This is further supported by the lack of effect of selective IKACh inhibitors on ventricular repolarisation in in vitro (Cha et al., 2006) and in vivo dog studies (Hashimoto et al., 2006; Hashimoto et al., 2008; Machida et al., 2011). The predominant expression of IKACh in the atria cf. the ventricle provides a mechanism to modulate atrial repolarisation without interfering with ventricular repolarisation and potentially inducing fatal ventricular arrhythmia (Hashimoto et al., 2006).

### (4) Constitutive-Activation of IKACh in Chronic AF:

The carbachol-induced IKACh recorded from atrial myocytes from patients with chronic AF is smaller than those from patients in sinus rhythm, a phenomenon initially thought to be due to decreased Kir3.4 mRNA and protein levels (Bosch et al., 1999; Brundel et al., 2001a; Brundel et al., 2001b; Dobrev et al., 2001). However, it was later demonstrated that the blunted response to carbachol is due to IKACh being constitutively active in the absence of agonist (Dobrev et al., 2005). Similar observations have also been reported in the atria and pulmonary vein in the tachypaced-dog model of AF (Cha et al., 2006; Ehrlich et al., 2004; Voigt et al., 2008; Makary et al., 2011). Ionic remodeling (for review see (Schotten et al., 2011; Workman et al., 2008), including the constitutive activation of IKACh, contributes to the shortening of action potential duration observed in chronic AF human patients (Dobrev et al., 2001; Dobrev et al., 2005; Bosch et al., 1999; Wettwer et al., 2004) and tachypaced dog atrial myocytes (Ehrlich et al., 2004; Ehrlich et al., 2007; Cha et al., 2006), which, in turn, causes a reduction in the atrial effective refractory period (Brundel et al., 2002b; Brundel et al., 2002a; Workman et al., 2008) predisposing to the generation of arrhythmias. In addition, the heterogeneous distribution (Gaborit et al., 2007a; Lomax et al., 2003; Sarmast et al., 2003; Voigt et al., 2010b) of constitutively active IKACh (Dobrev et al., 2005; Cha et al., 2006; Ehrlich et al., 2004) across the atria is expected to increase the dispersion of atrial repolarization/refractoriness (Liu & Nattel, 1997; Kabell et al., 1994; Schauerte et al., 2000; Chiou et al., 1997) and in turn increase vulnerability to transient atrial arrhythmias (Liu & Nattel, 1997; Kabell et al., 1994). Pharmacological studies have shown that selective inhibition of IKACh has as a more pronounced prolonging effect on action potential duration in the remodeled dog atria (Cha et al., 2006; Ehrlich et al., 2007). Prolonging the action potential duration by inhibiting IKACh or the constitutive IKACh could present safer pharmacological interventions for protecting against atrial arrhythmias such as chronic atrial fibrillation and atrial flutter compared to traditional class III antiarrhythmics by prolonging the atrial refractory period while leaving ventricular refractoriness unaltered (Cha et al., 2006; Tanaka & Hashimoto, 2007; Hashimoto et al., 2007; Machida et al., 2011).

### (5) IKACh Inhibitors in AF:

Class III antiarrhythmics have been widely reported as a preferred method for treating cardiac arrhythmias (Colatsky et al., 1990). Traditional and novel class III antiarrhythmic potassium channel blockers have been reported to have a mechanism of action that includes the direct modulation of Kir3.1/3.4 or IKACh. The known antiarrhythmics dronedarone (Altomare et al., 2000; Guillemare et al., 2000), amiodarone (Watanabe et al., 1996; Guillemare et al., 2000), propafenone (Voigt et al., 2010a) and flecainide (Voigt et al., 2010a), ibutilide (Borchard et al., 2005) quinidine (Kurachi et al., 1987; Hara & Kizaki, 2002), verapamil (Hibino et al., 2010), AVE0118 (Gögelein et al., 2004; Voigt et al., 2010a) NIP-142 (Matsuda et al., 2006; Hashimoto et al., 2007; Tanaka & Hashimoto, 2007), NIP-151 (Hashimoto et al., 2008), NTC-801 (Machida et al., 2011) have all been reported as potassium channel blockers of Kir3.1/3.4 or IKACh in atrial myocytes. A benzopyran derivative, NIP-142, preferentially blocks Kir3.1/3.4 with selectivity over other cardiac channels, prolongs the atrial refractory period and terminates atrial fibrillation and flutter in in vivo canine models (Nagasawa et al., 2002; Tanaka & Hashimoto, 2007). From the same chemical class, both NIP-151 and NTC-801 are highly selective IKACh inhibitors and have been shown to be effective in terminating AF in the vagal-induced and aconitine-induced canine models of AF (Hashimoto et al., 2008; Machida et al., 2011). The latter, NTC-801, has also been shown to prevent the induction of AF in an atrial-tachypacing dog model of persistent AF (AT-AF) (Machida et al., 2011) in which the atria exhibit electrical remodeling akin to chronic AF in man (Cha et al., 2006; Ehrlich et al., 2004; Voigt et al., 2008; Makary et al., 2011). The selective IKACh inhibitor peptide tertiapin (Jin & Lu, 1998; Drici et al., 2000) has also been shown to be effective in terminating AF in both vagal-induced and acontine-induced canine models of AF (Hashimoto et al., 2006). None of the agents were shown to affect ventricular repolarisation (QTc or VERP) at therapeutically relevant doses. These data support the utility of IKACh inhibitors for the cardioversion and prevention of recurrence of supraventricular arrhythmias such as AF and atrial flutter without effecting ventricular function. A combination of anti-arrhythmics with other ion channel modulating drugs may also provide greater (synergistic) benefit in the treatment of atrial arrhythmias as shown for the non-selective anti-arrhythmics drugs amiodarone/dronedarone and ranolozine (Burashnikov et al., 2010; Sicouri et al., 2009) and the combination of the IKr inhibitor sotalol with an IKur inhibitor BMS-394136 (Sun et al., 2010). As such, the combination of a selective IKACh inhibitor with other ion channel or ion exchanger modulating drugs could provide added clinical benefit.

### (6) IKACh Inhibition in Stroke Prevention in AF:

Atrial fibrillation is associated with a 5-fold increased risk for stroke and in the United States approximately 15% to 25% of all strokes can be attributed to AF (Steinberg, 2004). Regardless of the approach to arrhythmias treatment (rate, rhythm, ablation), the prevention of thromboembolism is a cornerstone of clinical treatment of atrial arrhythmias. Constitutive activation of IKACh has been reported to contribute to the contractile deficit associated with AF in the tachypaced-atrial dog model of AF Inhibition of IKACh could be a novel target to prevent hypocontractility-related thrombo-embolic complications (Koo et al., 2010). IKACh inhibitors alone or in combination with other anti-platelet or anti-coagulant therapies may significant reduce the risk of stroke and thromboembolism in AF.

### (7) Role of Autonomic System in AF:

Clinical (Coumel, 1994; Coumel, 1996; Pappone et al., 2004; Tan et al., 2006; Yamashita et al., 1997; Huang et al., 1998) and experimental (Liu & Nattel, 1997; Ogawa et al., 2007; Sharifov et al., 2004; Jayachandran et al., 2000; Scherlag et al., 2005; Horikawa-Tanami et al., 2007; Po et al., 2006) observations highlight the importance of the autonomic nervous system and in particular parasymthpathetic/vagal activation in AF. The electrophysiologic substrate of AF is often latent until vagal activation which is sufficient to induce and maintain AF via IKACh activation. IKACh inhibitors are expected to be effective in the treatment of paroxysmal AF with a neurogenic (vagal) component.

### (8) Autonomic System in the Initiation of AF:

Ectopic activity arising from the pulmonary veins and sleeves (PV) has been shown to play a prominent role in the initiation and maintenance of AF (Haissaguerre et al., 1998; Pappone et al., 2000). Pulmonary vein isolation is a procedure used frequently to eliminate the triggers arising from the pulmonary veins. Electrical activity, originating from PV sleeves following parasympathetic and/or sympathetic stimulation, has been proposed as a potential trigger in the initiation of AF (Burashnikov & Antzelevitch, 2006; Patterson et al., 2005; Patterson et al., 2006; Wongcharoen et al., 2007; Lo et al., 2007). Studies in animal models have shown an increase in the time-dependent IKACh in the pulmonary sleeve of the AT-AF dog (Ehrlich et al., 2004). Autonomic nerve stimulation reduces PV-sleeve action potential duration and causes triggered PV firing that is suppressed by muscarinic cholinergic receptor blockade (Patterson et al., 2005). Fibrillatory cycle length shortening in response to vagal stimulation points to ACh effects on PV drivers (Takahashi et al., 2006). Thus, inhibition of IKACH could remove vagally enhanced PV drivers that initiate and maintain AF.

### (9) Autonomic Nervous System in Atrial Remodeling:

Auto-antibodies to the muscarinic M2 receptor have been shown to increase expression of Kir3.1 and Kir3.4 mRNA and Kir3.4 protein in the rabbit heart, resulting in both electrical and structural remodeling creating a substrate for AF (Hong et al., 2009). Increased vagal-nerve activity has been shown to promote atrial electrical remodeling in atrial tachypaced dogs; this effect was partially revered by atropine and fully reversed by a combination of cholinergic block and a vasoactive intestinal polypeptide (VIP) antagonist (Yang et al., 2011). Clinical studies have also shown that parasympathetic block may promote the recovery from AERP shortening associated with rapid atrial pacing (Miyauchi et al., 2004). Although the mechanism that underlies these observations is not fully elucidated, inhibition of IKACH alone or in combination with other agents could prevent or reverse atrial remodeling associated with AF.

Beyond use in the treatment of atrial arrhythmias, Kir3.1/3.4 inhibitors may have utility in a number of other indications:

### (1) IKACh and Sinoatrial and Atrioventricular Node Function:

Acetylcholine (ACh) is an important neuromodulator of cardiac function that is released upon stimulation of the vagus nerve. Negative chronotropic and dromotropic effects are cardiovascular features associated with ACh release upon parasympathetic stimulation. In the mammalian heart, cholinergic parasympathetic fibres are extensively distributed to the sinus node, to the atria and to the atrioventricular (AV) node. Vagal stimulation produces a negative chronotropic and dromotropic effect on the heart and can induce or predispose to atrial arrhythmias due to shortening of the atrial ERP. Vagal stimulation increases AV-ERP (ALANIS et al., 1958; ALANIS et al., 1959), prolongs atrial conduction time (Martin, 1977) and produces a negative dromotropic effect. Selective inhibition of IKACh with tertiapin has been shown to inhibit the dromotropic and blunts the chronotropic effects of ACh on the heart and relieve AV block (Drici et al., 2000). The abundance of Kir3.1 and Kir3.4, is reported to be equal in the sinus node and atrial muscle (Tellez et al., 2006). Activation of IKACh causes decreased spontaneous activity, hyperpolarization of the maximum diastolic potential, and a decrease in the diastolic depolarization rate of the SA node contributing to the negative chronotropic effect of ACh (Dobrzynski et al., 2007; Han & Bolter, 2011; Rodriguez-Martinez et al., 2011). Atrial fibrillation is associated with structure and ionic remodelling in the atria (for review see (Schotten et al., 2011; Workman et al., 2008) and damage to the SAN (Thery et al., 1977). Clinical studies have shown that sick sinus syndrome is frequently associated with AF and atrial flutter (Ferrer, 1968; Gomes et al., 1981). Sinoatrial node dysfunction is a heterogeneous disorder of unknown etiology characterized by a variety of supraventricular arrhythmias with symptoms of persistent bradycardia, tachycardia, syncope, palpitations, and dizziness. The mechanism underlying the abnormal rhythm is incompletely understood. However, atropine, a muscarinic antagonist, is used in the treatment of sick sinus syndrome. However, side-effects preclude its long term use (1973). Taken together, these data highlight both the presence and functional importance of IKACh in the SAN and AVN and indicate the potential of an IKACh inhibitor to modulate AV conduction in setting of hypervagotony or early inferior myocardial infarctions (Drici et al., 2000) and provide a novel mechanism in the treatment of sinus node dysfunction.

### (2) Kir3.1/3.4 Inhibitors and Prevention of Thromboembolism:

Current approaches to the prevention of thromboembolism include the use of anti-platelet therapy (e.g. aspirin) or anticoagulation therapy including the use vitamin K antagonist warfarin, and oral agents, including direct thrombin inhibitors such as dabigatran, ximelagatran and factor Xa inhibitors such as apixaban, rivaroxaban, and edoxaban, betrixaban and YM150 (for review see (Ezekowitz et al., 2010)). Damaged blood vessels, red blood cells and platelets release ADP and induce platelet aggregation. Pathological thrombosis formation can lead to vascular occlusion, resulting in ischemic insults. The platelet ADP receptor designated P2Y12, the target of the antithrombotic agents like clopidogrel, activates Kir3.x channels via Gi/o proteins (Hollopeter et al., 2001). Human platelets have been shown to express both Kir3.1 and Kir3.4 protein by Western blot (Shankar et al., 2004). Kir3.1/3.4 inhibitors, such as SCH23390 and ethosuximide, can inhibit ADP- and thrombin-mediated platelet aggregation (Shankar et al., 2004; Kobayashi et al., 2009). Therefore, Kir3.1/3.4 inhibitors may be effective for preventing thrombosis and thromboembolic diseases including stroke, myocardial infarction and peripheral vascular diseases (Kobayashi & Ikeda, 2006).

### (3) Kir3.4 and Pancreatic Function:

Although predominantly expressed in the heart Kir3.4 has been cloned from the human pancreas (Chan et al., 1996) and has been detected in α, β, δ cells of the mouse pancreas (Yoshimoto et al., 1999; Ferrer et al., 1995; Iwanir & Reuveny, 2008). Electrophysiological studies have shown that somatostatin and α2-adrenoceptor agonists activate sulfonylurea-insensitive K⁺ channels by a G protein-dependent mechanisms, and thereby inhibit activity of Kir3.4-expressing β-cells (Rorsman et al., 1991), (Yoshimoto et al., 1999), suggesting that activation of Kir3 channels may inhibit insulin secretion. Additionally, somatostatin released from 6 cells activates Kir3 channels in glucagon-expressing α cells (Yoshimoto et al., 1999). The adrenaline-induced hyperpolarisation of mouse pancreatic cells has been shown to be a tertiapin-sensitive inwardly-rectifying potassium current (Iwanir & Reuveny, 2008). Therefore, pancreatic Kir3.4 channels may be related to control of pancreatic hormone secretion and have utility in the treatment of diabetes mellitus alone or in combination with sulfonylureas and other oral agents (Kobayashi & Ikeda, 2006).

### (4) Kir3.1/3.4 in the Central Nervous System:

In addition to expression in the heart, Kir3.1 and Kir3.4 mRNA have been detected in the parts of the brain (Wickman et al., 2000; Mark & Herlitze, 2000; Hibino et al., 2010). A number of psychotropic and antidepressant drugs have been shown to inhibit the Kir3.1/3.4 channels including paroxetine (Kobayashi et al., 2006), fluoxetine (Kobayashi et al., 2003), reboxetine (Kobayashi et al., 2010), atomoxetine (Kobayashi et al., 2010), mipramine, desipramine, amitriptyline, nortriptyline, clomipramine, maprotiline, citalopram (Kobayashi et al., 2004), and ethosuximide (Kobayashi et al., 2009). This suggests that the Kir3.x inhibition may underlie some of the therapeutic effects related to the CNS. As such, Kir3.1/3.4 inhibits may have utility in the treatment of neurological and neuropsychiatric disorders diseases including pain, depression, anxiety, attention-deficit/hyperactivity disorder, and epilepsy.

### (5) Kir3.1/3.4 and Pituitary Function:

Kir3.1 and Kir3.4 have been detected in the pituitary cells of the rat (Gregerson et al., 2001; Wulfsen et al., 2000) where they potentially play a critical role in excitation-secretion coupling. As such, Kir3.1/3.4 inhibitors could be used to modulate neuro-endocrine function and the secretion of pituitary hormones. However, corroborative data in man is currently lacking.

### (6) Kir3.1/3.4 and Cancer:

In addition, other reports have cloned Kir3.1 and Kir3.4 from human breast cancer cell line (Wagner et al., 2010) and suggest they may be involved in cellular signaling and cancer (Dhar & Plummer, III, 2006; Plummer, III et al., 2004). Although additional data are required to establish a causal link, targeting Kir3.1/3.4 could be useful in the treatment of breast cancer.

Nissan Chemical Industries have reported a series of substituted benzopyrans as atrial-specific antiarrythmics.

In WO 01/21610 Nissan discloses a series of benzopyran derivatives which are claimed to increase the functional refractory period in an ex vivo preparation of guinea pig atrial tissue with potential use as atrial-specific antiarrythmics.

In WO 02/064581, WO 03/000675 and WO 2005/080368 Nissan discloses a series of 4-amino substituted benzopyran derivatives which are claimed to selectively prolong the atrial refractory period in an in vivo dog model of vagal-induced atrial fibrillation with potential use as atrial-specific antiarrythmics.

In WO 2008/0004262 Nissan discloses a series of fused tricyclic benzopyran derivatives which are claimed to selectively prolong the atrial refractory period in an in vivo dog model of vagal-induced atrial fibrillation with potential use as atrial-specific antiarrythmics.

The above Nissan patents do not specify a biological target, but in subsequent publications (Hashimoto et al, 2008) compounds of these documents have been disclosed as blockers of the Kir3.1/3.4 channel and the IKACh cardiac current.

WO 2010/0331271 discloses a series of derivatives of the flavone acacetin which are claimed inter alia as blockers of the cardiac acetylcholine-activated current (IKACh) with potential use as atrial-specific antiarrythmics.

In WO 2009/104819 Otsuka Pharmaceuticals discloses a series of benzodiazepine derivatives which are claimed as blockers of the Kir3.1/3.4 channel with potential use as atrial-specific antiarrythmics.

Thienopyrazoles have been shown to have activity against voltage-gated and ligand-gated ion channels.

Akritopolou-Zanze et al (2006) disclose a series of thieno[2,3-c]pyrazoles as sub-micromaolar inhibitors of KDR kinase.

Brotherton-Pleiss et al (2010) and the related patent application US2007/0037974 disclose a series of thieno[2,3-c]pyrazoles as potent and selective analogues of the P2X3 receptor and identify a lead compound RO-85 from this series.

WO2011/058766 (Raqualia Pharmaceuticals) discloses a series of aryl carboxamides, including a thieno[2,3-c]pyrazole as blockers of TTX-sensitive sodium channels for the treatment of neuropathic pain.

Thienopyrazoles, thienooxazoles and thienopyrroles have been shown to have activity against other biological targets and disease areas.

Binder et al (1987) disclose a series of thieno[2,3-c]oxazoles as analogues of the anticonvulsant AD-810, which were inactive in a mouse electroshock assay.

EP1775298 (Daiichi Asubio Pharma) discloses a series of thieno[2,3-c]pyrazoles as inhibitors of PDE7 for the treatment of immunological disorders.

WO2005/026984 (Aventis) discloses a series of thieno[2,3-c]pyrazoles, which exhibit anticancer properties via inhibition of certain kinases.

US2011/0152243 (Abbott) discloses a series of substituted thienopyrroles with kinase inhibitory activity for the treatment of cancer.

US2005/074922 (Pharmacia) discloses a series of thieno[2,3-c]pyrazoles with inhibitory activity against Aurora kinase for the treatment of cancer.

WO2011/006066 (Ironwood Pharmaceuticals) discloses a series of thieno[2,3-b]pyrroles as agonists of the cannabinoid receptor.

### DISCLOSURE OF THE INVENTION

A first aspect of the invention provides a compound of formula (I)

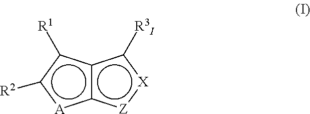

or a pharmaceutically acceptable derivative thereof, wherein:
A is O or S;
X is selected from N, O, CR³_{II} and NR³_{IV};
Z is selected from N, O, CR³_{III} and NR³_{V};
R¹ is selected from H, optionally substituted alkyl, optionally substituted cycloalkyl, optionally substituted aryl, and optionally substituted heteroaryl;

R² is selected from H, halo, —CN, trifluoromethyl, optionally substituted alkyl, optionally substituted alkoxy, —NR⁴R⁵, —NR⁶C(O)R⁷, —NR⁶S(O)₂R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴;
R³_{I} is selected from H, halo, —CN, trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, —C≡C-J, optionally substituted cycloalkyl-J and —(NR^{a}R^{b})-J;
Each of R³_{II} and R³_{III} is independently selected from H, halo, —CN, trifluoromethyl, optionally substituted alkyl, optionally substituted alkoxy, optionally substituted heterocycloalkoxy, optionally substituted heterocycloalkylalkyl, —NR⁶C(O)R⁷, —NR⁶S(O)₂R⁷, —S(O)₂NR⁴R⁵, —CONR⁴R⁵, optionally substituted -alkylene-CONR⁴R⁵, —CO₂R⁷, —SO₂R⁷, —NR¹⁰R¹¹, —C≡C-J, optionally substituted cycloalkyl-J and —(NR^{a}R^{b})-J;

Each of R³_{IV} and R³_{V} is independently selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, —C≡C-J, and optionally substituted cycloalkyl-J;
provided that at least one of R³_{I}, R³_{II} and R³_{III} is present as —C≡C-J, optionally substituted cycloalkyl-J or —(NR^{a}R^{b})-J, or at least one of R³_{IV} and R³_{V} is present as —C≡C-J or optionally substituted cycloalkyl-J;
wherein R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—;
J is selected from H and —(CR¹²R¹³)_{q}-L-M-W,
   wherein
q is 0, 1 or 2;
L is —O— or —N(G)-; and
G is selected from hydrogen, optionally substituted alkyl and optionally substituted cycloalkyl;
M is —(CR¹²R¹³)ₜ—;
t is 0, 1, 2 or 3;
W is selected from the group consisting of optionally substituted alkyl, optionally substituted alkoxy, optionally substituted alkenyl, optionally substituted cycloalkyl, optionally substituted heterocycloalkyl, optionally substituted aryl, optionally substituted heteroaryl and —NR⁸R⁹,
wherein when W is optionally substituted cycloalkyl it may optionally be bridged by a bond or optionally substituted C₁₋₂alkylene, and
wherein when W is optionally substituted heterocycloalkyl it may optionally be bridged by a bond, optionally substituted C₁₋₂alkylene, —NR⁶—, —O—, or —S(O)_{z}—;
alternatively, when L=—N(G)-, L, G, M and W may be linked to form an optionally substituted heterocycloalkyl, an optionally substituted heterocycloalkenyl, or an optionally substituted heteroaryl;
z is 0, 1 or 2;
R⁴ and R⁵ are, at each instance, independently selected from H, optionally substituted alkyl, optionally substituted aryl, optionally substituted heteroaryl, and optionally substituted cycloalkyl, or are linked to form an optionally substituted heterocycloalkyl;
R⁶ and R⁷ are, at each instance, independently selected from H and optionally substituted alkyl, or are linked to form an optionally substituted heterocycloalkyl;
R⁸ and R⁹ are, at each instance, independently selected from H, optionally substituted alkyl, optionally substituted aryl, optionally substituted heterocycloalkyl, optionally substituted heteroaryl, and optionally substituted cycloalkyl;
R¹⁰ and R¹¹ are, at each instance, independently selected from H, optionally substituted alkyl, optionally substituted aryl, optionally substituted heterocycloalkyl, optionally substituted heteroaryl, and optionally substituted cycloalkyl;
R¹² and R¹³ are, at each instance, independently selected from H, hydroxy, and optionally substituted alkyl, or may be linked to form an optionally substituted cycloalkyl ring, or may together form ═O; and
R¹⁴ is optionally substituted alkyl,
wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —C₁₋₆alkylene-NHSO₂C₁₋₆alkyl, —C₁₋₆alkylene-NC₁₋₆alkylSO₂Cl₁₋₆alkyl, —C₁₋₆alkylene-SO₂NH₂, —C₁₋₆alkylene-SO₂NHC₁₋₆alkyl, —C₁₋₆alkylene-SO₂N(C₁₋₆alkyl)₂, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment, A is S and Z is N. In a further embodiment, A is S and Z is NR³_{V}. In a further embodiment, X is N. In a further embodiment, R¹ is phenyl. In a further embodiment, R² is selected from H, trifluoromethyl, substituted alkyl, optionally substituted alkoxy, —NR⁴R⁵, —NR⁶C(O)R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴. In a further embodiment, R³_{I} is selected from trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, optionally substituted cycloalkyl-J and —(NR^{a}R^{b})-J. In a further embodiment, R³_{V} is selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, and optionally substituted cycloalkyl-J. In a further embodiment, R³_{V} is selected from H, optionally substituted alkyl, —C(O)R⁷, and —SO₂R⁷. In a further embodiment, R³_{I} is —(NR^{a}R^{b})-J and J is —(CR¹²R¹³)q-L-M-W. In a further embodiment, q is 0 or 1. In a further embodiment, q is 1. In a further embodiment, t is 0, 1 or 2. In a further embodiment, t is 2. In a further embodiment, L is O, or, in an alternative embodiment, L is —N(G)-. In a further embodiment, R¹² and R¹³ are, at each instance, H. In a further embodiment, W is optionally substituted heterocycloalkyl.

A second aspect of the invention provides a pharmaceutical composition comprising at least one compound of formula (I) and, optionally, one or more pharmaceutically acceptable excipients.

A third aspect of the invention provides a compound of formula (I) or a composition comprising at least one compound of formula (I) for use in therapy.

A fourth aspect of the invention provides a method for the treatment of a disease or condition that is mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, comprising administering to a subject an effective amount of at least one compound of formula (I) or composition comprising at least one compound of formula (I).

A fifth aspect of the invention provides a compound of formula (I) or a composition comprising at least one compound of formula (I) for use in a method for the treatment of a disease or condition that is mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, comprising administering to a subject an effective amount of at least one compound of formula (I) or composition comprising at least one compound of formula (I).

A sixth aspect of the invention provides the use of a compound of formula (I) for the manufacture of a medicament for use in the treatment of a disease or condition that is mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof.

As discussed above, inhibition of Kᵢᵣ3.1 and/or Kᵢᵣ3.4 (or heteromultimers thereof) has implications in:
the diagnosis and treatment of cardiovascular diseases, such as atrial fibrillation (AF), atrial flutter (AFL), atrioventricular (AV) dysfunction and sinoatrial node (SAN) dysfunction;
the prevention of recurrence of supraventriclar arrhythmias including AF and AFL;
the maintenance of sinus rhythm;
the termination and cardioversion of supraventriclar arrhythmias;
the treatment of sinus node dysfunction;
the treatment of AV node dysfunction, including AV block;
the treatment of conduction dysfunction;
the prevention or reversal of atrial structural and ionic remodeling;
the prevention of thrombosis, thromboembolism and thromboembolic diseases, such as stroke, myocardial infarction, and peripheral vascular diseases;
the improvement of cardiac contractility;
the treatment of metabolic diseases, such as diabetes mellitus;
the modulation of neuro-endocrine function;
the modulation of the secretion of pituitary hormones;
the treatment of neurological and neuropsychiatric disorders, such as pain, depression, anxiety, attention deficit/hyperactivity disorder and epilepsy; and
the treatment of cancer, such as breast cancer.

### DETAILED DESCRIPTION OF THE INVENTION

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

However, combinations of features are permissible only if such combinations result in stable compounds. Compounds of the invention are typically stable and isolatable at room temperature and pressure. A “stable” compound is sufficiently robust to survive isolation to a useful degree of purity from a reaction mixture, and formulation into an efficacious therapeutic agent.

As is clear from formula (I), the core ring system of the claimed compounds, which contains A, X and Z, is aromatic. Therefore, combinations of X and Z that result in non-aromatic rings are not covered by formula (I). Specifically:
if X is N or CR³_{II}, then Z is not N or CR³_{III}; and
if X is O or NR³_{IV}, then Z is not O or NR³_{V}.

In one embodiment, A is S. In another embodiment, A is O.

In one embodiment, Z is O and X is N. In another embodiment, Z is N and X is NR³_{IV}. In another embodiment, Z is NR³_{V} and X is N.

In a specific embodiment, A is S, Z is NR³_{V} and X is N, i.e. the compounds are thienopyrazoles.

In one embodiment, at least one of R³_{I}, R³_{II} and R³_{III} is present as optionally substituted cycloalkyl-J or —(NR^{a}R^{b})-J, and/or at least one of R³_{IV} and R³_{V} is present as optionally substituted cycloalkyl-J. In another embodiment, at least one of R³_{I}, R³_{II} and R³_{III} is present as —(NR^{a}R^{b})-J, and/or at least one of R³_{IV} and R³_{V} is present as optionally substituted cycloalkyl-J. In another embodiment, at least one of R³_{I}, R³_{II} and R³_{III} is present as —(NR^{a}R^{b})-J.

In one embodiment, R³_{I} is selected from H, halo, —CN, trifluoromethyl, optionally substituted alkyl, optionally substituted alkoxy, —NR⁶C(O)R⁷, —NR⁶S(O)₂R⁷, —S(O)₂NR⁴R⁵, —CONR⁴R⁵, —CO₂R⁷, —NR⁸R⁹, —C≡C-J, optionally substituted cycloalkyl-J and —(NR^{a}R^{b})-J. In another embodiment, R³_{I} is selected from trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, optionally substituted cycloalkyl-J and —(NR^{a}R^{b})-J. In another embodiment, R³_{I} is selected from H, —(NR^{a}R^{b})-J, optionally substituted cycloalkyl-J and —C≡C-J. In another embodiment, R³_{I} is selected from —(NR^{a}R^{b})-J, and —C≡C-J. In another embodiment, R³_{I} is selected from —(NR^{a}R^{b})-J, and optionally substituted cycloalkyl-J. In another embodiment, R³_{I} is —(NR^{a}R^{b})-J. In another embodiment, R³_{I} is —(NR^{a}R^{b})-J and J is (CR¹²R¹³)_{q}-L-M-W.

In one embodiment, each of R³_{II} and R³_{III} is independently selected from H, halo, —CN, trifluoromethyl, optionally substituted alkyl, optionally substituted alkoxy, —NR⁶C(O)R⁷, —NR⁶S(O)₂R⁷, —S(O)₂NR⁴R⁵, —CONR⁴R⁵, —CO₂R⁷, —SO₂R⁷, —NR¹⁰R¹¹, —C≡C-J, optionally substituted cycloalkyl-J and —(NR^{a}R^{b})-J. In another embodiment, each of R³_{II} and R³_{III} is independently selected from H, halo, —CN, trifluoromethyl, optionally substituted alkyl, optionally substituted alkoxy, optionally substituted heterocycloalkoxy, optionally substituted heterocycloalkylalkyl, —NR⁶C(O)R⁷, —NR⁶S(O)₂R⁷, —S(O)₂NR⁴R⁵, —CONR⁴R⁵, optionally substituted -alkylene-CONR⁴R⁵, —CO₂R⁷, —SO₂R⁷, —NR¹⁰R¹¹, optionally substituted cycloalkyl-J and —(NR^{a}R^{b})-J. In another embodiment, each of R³_{II} and R³_{III} is independently selected from H, halo, —CN, trifluoromethyl, optionally substituted alkoxy, optionally substituted heterocycloalkoxy, optionally substituted heterocycloalkylalkyl, —NR⁶C(O)R⁷, —NR⁶S(O)₂R⁷, —S(O)₂NR⁴R⁵, —CONR⁴R⁵, optionally substituted -alkylene-CONR⁴R⁵, —CO₂R⁷, —SO₂R⁷, —NR¹⁰R¹¹, —C≡C-J, optionally substituted cycloalkyl-J and —(NR^{a}R^{b})-J. In another embodiment, each of R³_{II} and R³_{III} is independently selected from H, —NR¹⁰R¹¹, —C≡C-J, optionally substituted cycloalkyl-J and —(NR^{a}R^{b})-J. In one embodiment, each of R³_{II} and R³_{III} is independently selected from H, —NR¹⁰R¹¹, —C≡C-J and —(NR^{a}R^{b})-J. In another embodiment, R³_{II} and R³_{III} are H. In another embodiment, R³_{II} and R³_{III} are independently selected from —C≡C-J, optionally substituted cycloalkyl-J and —(NR^{a}R^{b})-J. In another embodiment, R³_{II} and R³_{III} are selected from optionally substituted cycloalkyl-J and —(NR^{a}R^{b})-J. In another embodiment, R³_{II} and R³_{III} are —(NR^{a}R^{b})-J.

In one embodiment, each of R³_{IV} and R³_{V} is independently selected from H, —CN, trifluoromethyl, optionally substituted alkyl, —S(O)₂NR⁴R⁵, —CONR⁴R⁵, —CO₂R⁷, —C(O)R⁷, —SO₂R⁷, —C≡C-J, and optionally substituted cycloalkyl-J In another embodiment, each of R³_{IV} and R³_{V} is independently selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, and optionally substituted cycloalkyl-J. In another embodiment, each of R³_{IV} and R³_{V} is independently selected from H, —CN, trifluoromethyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, —C≡C-J, and optionally substituted cycloalkyl-J. In another embodiment, each of R³_{IV} and R³_{V} is independently selected from H, —C≡C-J, and optionally substituted cycloalkyl-J. In one embodiment, each of R³_{IV} and R³_{V} is independently selected from H, and —C≡C-J. In another embodiment, R³_{IV} and R³_{V} are independently selected from —C≡C-J, and optionally substituted cycloalkyl-J. In another embodiment, each of R³_{IV} and R³_{V} is independently selected from H, optionally substituted alkyl, —C(O)R⁷, and —SO₂R⁷. In another embodiment, R³_{V} is selected from H, optionally substituted alkyl, —C(O)R⁷, and —SO₂R⁷.

In one embodiment, R¹ is selected from optionally substituted alkyl, optionally substituted cycloalkyl, optionally substituted aryl, and optionally substituted heteroaryl In another embodiment, R¹ is selected from optionally substituted alkyl, optionally substituted heteroaryl and optionally substituted aryl. In another embodiment, R¹ is selected from optionally substituted alkyl and optionally substituted aryl. In another embodiment, R¹ is selected from optionally substituted heteroaryl and optionally substituted aryl. In another embodiment, R¹ is selected from optionally substituted alkyl and optionally substituted phenyl. In another embodiment, R¹ is selected from optionally substituted methyl, optionally substituted ethyl, optionally substituted i-propyl, and optionally substituted phenyl. In another embodiment, R¹ is selected from methyl, ethyl, i-propyl, and phenyl, wherein phenyl is optionally substituted by one or more of halo, —NO₂ and —SO₂N(C₁₋₆alkyl)₂. In another embodiment, R¹ is selected from methyl, ethyl, i-propyl, and phenyl, wherein phenyl is optionally substituted by one or more of F, —NO₂ and —SO₂NMe₂. In another embodiment, R¹ is optionally substituted phenyl. In another embodiment, R¹ is phenyl. In another embodiment, R¹ is substituted phenyl. In another embodiment, R¹ is selected from methyl, ethyl and i-propyl. In embodiments in which R¹ is substituted phenyl, it may be substituted at the 2-, 3-, 4-, 5- and/or 6-position(s). In one embodiment, R¹ is 2-substituted phenyl and in a further embodiment, the 2-substituent is methoxy.

R² is selected from H, halo, —CN, trifluoromethyl, optionally substituted alkyl, optionally substituted alkoxy, —NR⁴R⁵, —NR⁶C(O)R⁷, —NR⁶S(O)₂R⁷, —S(O)₂NR⁴R⁵, —CONR⁴R⁵, and —CO₂R⁷. In one embodiment, R² is selected from halo, —CN, trifluoromethyl, optionally substituted alkyl, optionally substituted alkoxy, —NR⁴R⁵, —NR⁶C(O)R⁷, —NR⁶S(O)₂R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴. In another embodiment, R² is selected from H, halo, —CN, trifluoromethyl, optionally substituted alkyl, optionally substituted alkoxy, —NR⁴R⁵, —NR⁶C(O)R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴. In another embodiment, R² is selected from H, trifluoromethyl, substituted alkyl, optionally substituted alkoxy, —NR⁴R⁵, —NR⁶C(O)R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴. In another embodiment, R² is selected from H, halo, —CN, optionally substituted alkyl, —NR⁴R⁵, —NR⁶C(O)R⁷, and —CONR⁴R⁵. In another embodiment, R² is selected from H, halo, —CN, optionally substituted methyl, ethyl, and i-propyl, —NR⁴R⁵, —NR⁶C(O)R⁷, and —CONR⁴R⁵. In another embodiment, R² is selected from H, bromo, —CN, methyl, ethyl, i-propyl, —NR⁴R⁵, —NR⁶C(O)R⁷, and —CONR⁴R⁵. In another embodiment, R² is selected from H, —NR⁶C(O)R⁷, and —CONR⁴R⁵. In another embodiment, R² is selected from H and —CONR⁴R⁵. In another embodiment, R² is H. In one embodiment, optionally substituted oxazolinyl is optionally substituted 2-oxazolinyl.

In a specific embodiment, R¹ is phenyl and R² is H.

R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—. J may be attached to any atom on the ring or, if present, the bridge. In one embodiment, NR^{a}R^{b} forms an optionally bridged, optionally substituted heterocycloalkyl selected from the group consisting of azetidinyl, pyrrolidinyl, piperidinyl, morpholinyl, tetrahydro-1,3-oxazinyl, piperazinyl, hexahydropyrimidinyl, 1,4-thiazanyl, azepanyl, 1,4-oxaazepanyl, 1,4-thieazepanyl and 1,4-diazepanyl. In one embodiment, NR^{a}R^{b} forms an optionally bridged, optionally substituted ring of formula (II):

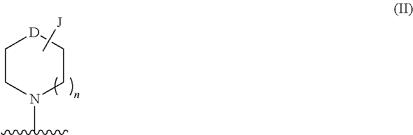

wherein
n is 0, 1 or 2;
D is selected from —CH₂—, —CHJ-, —O—, —N(H)— and —N(J)-.

In one embodiment, D is selected from —CHJ- and —N(J)-. In one embodiment, n is 0 or 1. In one embodiment, n is 1. In another embodiment, n is 0.

In one embodiment, NR^{a}R^{b} is optionally bridged by bond, —CH₂—, —C₂H₄— or —CHJ-. In another embodiment, NR^{a}R^{b} is optionally bridged by bond, —CH₂— or —CHJ-. In another embodiment, NR^{a}R^{b} is bridged by bond, —CH₂—, —C₂H₄— or —CHJ-. In another embodiment, NR^{a}R^{b} is bridged by bond, —CH₂— or —CHJ-. In another embodiment, NR^{a}R^{b} is not bridged.

In one embodiment, NR^{a}R^{b} is selected from optionally substituted pyrrolidinyl, optionally substituted piperidinyl, optionally substituted morpholinyl, optionally substituted piperazinyl, optionally substituted azabicyclohexanyl, optionally substituted azabicycloheptanyl, and optionally substituted azabicyclooctanyl. In another embodiment, NR^{a}R^{b} is selected from optionally substituted pyrrolidinyl, optionally substituted piperidinyl, optionally substituted morpholinyl, optionally substituted piperazinyl, optionally substituted azabicyclo[3.1.0]hexanyl, optionally substituted azabicyclo[2.2.1]heptanyl, and optionally substituted azabicyclo[3.2.1]octanyl. In another embodiment, NR^{a}R^{b} is selected from optionally substituted pyrrolidinyl, optionally substituted piperidinyl, optionally substituted morpholinyl, optionally substituted piperazinyl, optionally substituted 3-azabicyclo[3.1.0]hexanyl, optionally substituted 2-azabicyclo[2.2.1]heptanyl, and optionally substituted 8-azabicyclo[3.2.1]octanyl. In another embodiment, NR^{a}R^{b} is selected from optionally substituted pyrrolidinyl, piperidinyl, morpholinyl, piperazinyl, and 3-azabicyclo[3.1.0]hexanyl. In another embodiment, NR^{a}R^{b} is selected from pyrrolidinyl, piperidinyl, piperazinyl, and 3-azabicyclo[3.1.0]hexanyl. In another embodiment, NR^{a}R^{b} is selected from pyrrolidinyl, piperidinyl, and piperazinyl. In another embodiment, NR^{a}R^{b} is selected from pyrrolidinyl and piperidinyl. In one embodiment, NR^{a}R^{b} is pyrrolidinyl. In another embodiment, NR^{a}R^{b} is piperidinyl.

J may be attached to any atom on the ring or, if present, the bridge. In one embodiment, NR^{a}R^{b} is pyrrolidinyl and J is present at the 3-position. In another embodiment, NR^{a}R^{b} is piperidinyl and J is present at the 4-position.

In one embodiment, J is —(CR¹²R¹³)_{q}-L-M-W. In another embodiment, J is H. In another embodiment, if more than one J group is present, then, in at least one instance J is present as —(CR¹²R¹³)_{q}-L-M-W.

In one embodiment, q is 0 or 1. In one embodiment, q is 1 or 2. In another embodiment, q is 0 or 2. In another embodiment, q is 0. In another embodiment, q is 1. In another embodiment, q is 2. In another embodiment, q is 1 or 2 and R¹² and R¹³ are independently selected from H and alkyl. In another embodiment, q is 1 or 2 and R¹² and R¹³ are both H. In another embodiment, q is 1 and R¹² and R¹³ are both H.

In one embodiment, L is O. In another embodiment, L is —N(G)-.

In one embodiment, L is —N(G)- and L, G, M and W may be linked to form an optionally substituted heterocycloalkyl. In one embodiment, L is —N(G)- and L, G, M and W are linked to form an optionally substituted heterocycloalkyl. In another embodiment, L is —N(G)- and L, G, M and W are linked to form optionally substituted azetidinyl, optionally substituted pyrrolidinyl, optionally substituted piperidinyl or optionally substituted morpholinyl. In another embodiment, L is —N(G)- and L, G, M and W are linked to form azetidinyl, pyrrolidinyl, piperidinyl or morpholinyl, wherein each of pyrrolidinyl, piperidinyl and morpholinyl is optionally substituted by one or more groups selected from halo, trihalomethyl, —OH, —C₁₋₆alkyl, —O—C₁₋₆alkyl, —N(C₁₋₆alkyl)₂, —C₁₋₆alkylene-OH, aryl, haloaryl, —C(═O)NH₂ and —C₃₋₆heterocycloalkyl. In another embodiment, L is —N(G)- and L, G, M and W are linked to form pyrrolidinyl, piperidinyl or morpholinyl substituted by one or more groups selected from pyrrolidinyl, —OH, —F, -Me, —OMe, —CH₂OH, —CF₃, —NMe₂, phenyl, F-phenyl, —CONH₂.

In one embodiment, G is selected from hydrogen, and optionally substituted alkyl. In another embodiment, G is selected from H, optionally substituted methyl and optionally substituted ethyl. In another embodiment, G is selected from H, methyl and ethyl, wherein ethyl is optionally substituted by —OH or —O—C₁₋₆alkyl. In another embodiment, G is selected from H, methyl and ethyl, wherein ethyl is optionally substituted by —OH or —O-Me. In another embodiment, G is selected from H and methyl.

In one embodiment, t is 0, 1 or 2. In another embodiment, t is 0. In another embodiment, t is 1. In another embodiment, t is 2. In another embodiment, t is 3. In another embodiment, M is selected from bond, —(CH₂)—, —(CH₂)₂—, —(CH₂)₃—, -cycloalkyl-, —CHOH—CH₂—, —CH₂—CHOH—, —CH₂—C(alkyl)₂-, —(CH₂)—C(═O)—, —C(═O)—(CH₂)—. In another embodiment, M is selected from bond, —(CH₂)—, —(CH₂)₂—, —(CH₂)₃—, -cyclopentyl-, —CHOH—CH₂—, —CH₂—C(Me)₂-, —(CH₂)—C(═O)—. In another embodiment, M is selected from bond, —(CH₂)—, —(CH₂)₂— and —(CH₂)₃—.

In one embodiment, W is selected from the group consisting of substituted alkyl, alkoxy, alkenyl, cycloalkyl, optionally substituted heterocycloalkyl, aryl, heteroaryl. In another embodiment, W is selected from substituted alkyl, alkoxy, cyclopropyl, cyclobutyl, optionally substituted pyrrolidinyl, optionally substituted piperidinyl, optionally substituted piperazinyl, optionally substituted morpholinyl, tetrahydrofuran, furan, thiophene, phenyl, and pyridine. In another embodiment, W is selected from alkyl substituted by one or more groups selected from halo, —OH, —NH₂, and —N(C₁₋₆alkyl)₂, alkoxy, cyclopropyl, cyclobutyl, pyrrolidinyl, piperidinyl, piperazinyl, morpholinyl, tetrahydrofuran, furan, thiophene, phenyl, and pyridine, wherein each of pyrrolidinyl, piperidinyl, piperazinyl, and morpholinyl is optionally substituted by one or more groups selected from halo, C₁₋₆alkyl, —C(═O)C₁₋₆alkyl, —CO₂C₁₋₆alkyl, —N(C₁₋₆alkyl)₂, —NHC(═O)C₁₋₆alkyl, —C(═O)NH₂, and ═O. In another embodiment, W is selected from alkyl substituted by one or more groups selected from —F, —OH, —NH₂, and —N(Me)₂, alkoxy, cyclopropyl, cyclobutyl, pyrrolidinyl, piperidinyl, piperazinyl, morpholinyl, tetrahydrofuran, furan, thiophene, phenyl, and pyridine, wherein each of pyrrolidinyl, piperidinyl, piperazinyl, and morpholinyl is optionally substituted by one or more groups selected from —F, -Me, -Et, -iPr, —C(═O)Me, —CO₂tBu, —NHC(═O)Me, —C(═O)NH₂, and ═O. In another embodiment, W is selected from cyclopropyl, cyclobutyl, pyrrolidinyl, and piperidinyl, wherein each of pyrrolidinyl and piperidinyl is optionally substituted by one of -Me, -Et and -iPr. In another embodiment, W is selected from pyrrolidinyl, and piperidinyl, wherein each of pyrrolidinyl and piperidinyl is optionally substituted by one of -Me, -Et and -iPr. In one embodiment, W is 1-methylpyrrolidin-2-yl.

In one embodiment, z is 0. In another embodiment, z is 1. In another embodiment, z is 2.

In one embodiment, R⁴ and R⁵ are, at each instance, independently selected from H and optionally substituted alkyl, or are linked to form an optionally substituted heterocycloalkyl. In another embodiment, R⁴ and R⁵ are, at each instance, independently selected from H, optionally substituted methyl, optionally substituted ethyl, optionally substituted i-propyl, and optionally substituted pyrrolidinyl. In another embodiment, R⁴ and R⁵ are, at each instance, independently selected from H, methyl, ethyl, i-propyl, and pyrrolidinyl optionally substituted by ═O.

R⁶ and R⁷ are, at each instance, independently selected from H and optionally substituted alkyl, or, in the groups —NR⁶C(O)R⁷, —NR⁶S(O)₂R⁷, may be linked to form an optionally substituted heterocycloalkyl.

In one embodiment, R⁶ is, at each instance, independently selected from H and optionally substituted alkyl. In another embodiment, R⁶ is H.

In one embodiment, R⁷ is, at each instance, independently selected from H and optionally substituted alkyl. In another embodiment, R⁷ is alkyl. In another embodiment, R⁷ is methyl.

In one embodiment, R⁸ and R⁹ are, at each instance, independently selected from optionally substituted alkyl, optionally substituted aryl, optionally substituted heteroaryl, and optionally substituted cycloalkyl. In another embodiment, R⁸ and R⁹ are, at each instance, independently selected from optionally substituted alkyl, and optionally substituted cycloalkyl. In another embodiment, R⁸ and R⁹ are, at each instance, independently selected from optionally substituted aryl, optionally substituted heteroaryl, and optionally substituted cycloalkyl.

In one embodiment, R¹⁰ and R¹¹ are, at each instance, independently selected from H, optionally substituted alkyl, optionally substituted aryl, optionally substituted heteroaryl, and optionally substituted cycloalkyl. In another embodiment, R¹⁰ and R¹¹ are, at each instance, independently selected from H, optionally substituted alkyl, and optionally substituted cycloalkyl. In another embodiment, R¹⁰ and R¹¹ are, at each instance, independently selected from H, optionally substituted methyl, optionally substituted ethyl, and optionally substituted i-propyl. In another embodiment, R¹⁰ and R¹¹ are, at each instance, independently selected from optionally substituted methyl, optionally substituted ethyl, and optionally substituted i-propyl. In another embodiment, R¹⁰ and R¹¹ are, at each instance, independently selected from H, methyl, ethyl, and i-propyl, wherein each of methyl, ethyl, and i-propyl is optionally substituted by one or more of —OH, —O—C₁₋₆alkyl, —C₃₋₆cycloalkyl, —C₃₋₆heterocycloalkyl and —C(═O)NH₂. In another embodiment, R¹⁰ and R¹¹ are, at each instance, independently selected from H, methyl, ethyl, and i-propyl, wherein each of methyl, ethyl, and i-propyl is optionally substituted by one or more of —OH, -OMe, cyclopropyl, pyrrolidinyl and —C(═O)NH₂. In another embodiment, R¹⁰ and R¹¹ are, at each instance, independently selected from H, methyl, ethyl, and i-propyl, wherein each of methyl, ethyl, and i-propyl is substituted by one or more of —OH, -OMe, cyclopropyl, pyrrolidinyl and —C(═O)NH₂. In one embodiment, R¹⁰ is H.

In one embodiment, R¹² is H and R¹³ is, at each instance, independently selected from hydroxy, and optionally substituted alkyl, or R¹² and R¹³ are linked to form an optionally substituted cycloalkyl ring, or together form ═O. In another embodiment, R¹² and R¹³ are, at each instance, independently selected from H, hydroxy, and optionally substituted alkyl. In another embodiment, R¹² and R¹³ are, at each instance, independently selected from H, hydroxy, optionally substituted methyl, and optionally substituted ethyl. In another embodiment, R¹² and R¹³ are, at each instance, H.

In one embodiment, R¹⁴ is alkyl. In another embodiment, R¹⁴ is methyl.

### Specific Embodiments

In one embodiment,
A is O or S;
X is selected from N, O, CR³_{II} and NR³_{IV};
Z is selected from N, O, CR³_{III} and NR³_{V};
   wherein if X is CR³_{II}, then Z is not CR³_{III};
R¹ is selected from H, optionally substituted alkyl, optionally substituted cycloalkyl, optionally substituted aryl, and optionally substituted heteroaryl;
R² is selected from H, halo, —CN, trifluoromethyl, optionally substituted alkyl, optionally substituted alkoxy, —NR⁴R⁵, —NR⁶C(O)R⁷, —NR⁶S(O)₂R⁷, —S(O)₂NR⁴R⁵, —CONR⁴R⁵, —CO₂R⁷;
R³_{I} is selected from H, halo, —CN, trifluoromethyl, optionally substituted alkyl, optionally substituted alkoxy, —NR⁶C(O)R⁷, —NR⁶S(O)₂R⁷, —S(O)₂NR⁴R⁵, —CONR⁴R⁵, —CO₂R⁷, —NR⁸R⁹, —C≡C-J, optionally substituted cycloalkyl-J and —(NR^{a}R^{b})-J;
Each of R³_{II} and R³_{III} is independently selected from H, halo, —CN, trifluoromethyl, optionally substituted alkyl, optionally substituted alkoxy, —NR⁶C(O)R⁷, —NR⁶S(O)₂R⁷, —S(O)₂NR⁴R⁵, —CONR⁴R⁵, —CO₂R⁷, —SO₂R⁷, —NR¹⁰R¹¹—C≡C-J, optionally substituted cycloalkyl-J and —(NR^{a}R^{b})-J;
Each of R³_{IV} and R³_{V} is independently selected from H, —CN, trifluoromethyl, optionally substituted alkyl, —S(O)₂NR⁴R⁵, —CONR⁴R⁵, —CO₂R⁷, —C(O)R⁷, —SO₂R⁷, —C≡C-J, and optionally substituted cycloalkyl-J;
provided that at least one of R³_{I}, R³_{II} and R³_{III} is present as —C≡C-J, optionally substituted cycloalkyl-J or —(NR^{a}R^{b})-J, or at least one of R³_{IV} and R³_{V} is present as —C≡C-J or optionally substituted cycloalkyl-J;
wherein R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—;
J is selected from H and —(CR¹²R¹³)_{q}-L-M-W,
   wherein
q is 0, 1 or 2;
L is —O— or —N(G)-; and
G is selected from hydrogen and optionally substituted alkyl;
M is —(CR¹²R¹³)ₜ—;
t is 0, 1, 2 or 3;
W is selected from the group consisting of optionally substituted alkyl, optionally substituted alkoxy, optionally substituted alkenyl, optionally substituted cycloalkyl, optionally substituted heterocycloalkyl, optionally substituted aryl, optionally substituted heteroaryl and —NR⁸R⁹,
wherein when W is optionally substituted cycloalkyl it may optionally be bridged by a bond or optionally substituted C₁₋₂alkylene, and
wherein when W is optionally substituted heterocycloalkyl it may optionally be bridged by a bond, optionally substituted C₁₋₂alkylene, —NR⁶—, —O—, or —S(O)_{z}—;
   alternatively, when L=—N(G)-, L, G, M and W may be linked to form an optionally substituted heterocycloalkyl, an optionally substituted heterocycloalkenyl, or an optionally substituted heteroaryl;
z is 0, 1 or 2;
R⁴ and R⁵ are, at each instance, independently selected from H, optionally substituted alkyl, optionally substituted aryl, optionally substituted heteroaryl, and optionally substituted cycloalkyl, or are linked to form an optionally substituted heterocycloalkyl;
R⁶ is, at each instance, independently selected from H and optionally substituted alkyl;
R⁷ is, at each instance, independently selected from H and optionally substituted alkyl;
R⁸ and R⁹ are, at each instance, independently selected from H, optionally substituted alkyl, optionally substituted aryl, optionally substituted heteroaryl, and optionally substituted cycloalkyl; and
R¹⁰ and R¹¹ are, at each instance, independently selected from H, optionally substituted alkyl, optionally substituted aryl, optionally substituted heteroaryl, and optionally substituted cycloalkyl; and
R¹² and R¹³ are, at each instance, independently selected from H, hydroxy, and optionally substituted alkyl, or may be linked to form an optionally substituted cycloalkyl ring, or may together form ═O,
wherein the optional substituents are independently selected from halo, trihalomethyl, trihaloethyl, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —SO₂NH₂, —SO₂NHC₁₋₆alkyl, —SO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, ═O, —N(C₁₋₆alkyl)₂, —C(═O)NH₂, —C(═O)NHC₁₋₆alkyl, —C(═O)N(C₁₋₆alkyl)₂, —C₁₋₆alkyl, —C₃₋₆cycloalkyl, —C₃₋₆heterocycloalkyl, aryl, haloaryl, —Z^{t}H, —Z^{t}—C₁₋₆alkyl, —C₁₋₆alkylene-Z^{t}H or —Z^{t}—C₃₋₆cycloalkyl, wherein Z^{t} is independently O, S, NH or N(C₁₋₆alkyl).

In one embodiment,
A is S;
X is N;
Z is NR³_{V};
R¹ is selected from H, optionally substituted alkyl, optionally substituted cycloalkyl, optionally substituted aryl, and optionally substituted heteroaryl;
R² is selected from H, halo, —CN, trifluoromethyl, optionally substituted alkyl, optionally substituted alkoxy, —NR⁴R⁵, —NR⁶C(O)R⁷, —NR⁶S(O)₂R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴;
R³_{I} is selected from H, halo, —CN, trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, —C≡C-J, optionally substituted cycloalkyl-J and —(NR^{a}R^{b})-J;
R³_{V} is selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, —C≡C-J, and optionally substituted cycloalkyl-J;
provided that R³_{I} is present as —C≡C-J, optionally substituted cycloalkyl-J or —(NR^{a}R^{b})-J, or R³_{V} is present as —C≡C-J or optionally substituted cycloalkyl-J;
wherein R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—;
J is selected from H and —(CR¹²R¹³)_{q}-L-M-W,
   wherein
q is 0, 1 or 2;
L is —O— or —N(G)-; and
G is selected from hydrogen, optionally substituted alkyl and optionally substituted cycloalkyl;
M is —(CR¹²R¹³)ₜ—;
t is 0, 1, 2 or 3;
W is selected from the group consisting of optionally substituted alkyl, optionally substituted alkoxy, optionally substituted alkenyl, optionally substituted cycloalkyl, optionally substituted heterocycloalkyl, optionally substituted aryl, optionally substituted heteroaryl and —NR⁸R⁹,
wherein when W is optionally substituted cycloalkyl it may optionally be bridged by a bond or optionally substituted C₁₋₂alkylene, and
wherein when W is optionally substituted heterocycloalkyl it may optionally be bridged by a bond, optionally substituted C₁₋₂alkylene, —NR⁶—, —O—, or —S(O)_{z}—; alternatively, when L=—N(G)-, L, G, M and W may be linked to form an optionally substituted heterocycloalkyl, an optionally substituted heterocycloalkenyl, or an optionally substituted heteroaryl;
z is 0, 1 or 2;
R⁴ and R⁵ are, at each instance, independently selected from H, optionally substituted alkyl, optionally substituted aryl, optionally substituted heteroaryl, and optionally substituted cycloalkyl, or are linked to form an optionally substituted heterocycloalkyl;
R⁶ is, at each instance, independently selected from H and optionally substituted alkyl;
R⁷ is, at each instance, independently selected from H and optionally substituted alkyl;
R⁸ and R⁹ are, at each instance, independently selected from H, optionally substituted alkyl, optionally substituted aryl, optionally substituted heterocycloalkyl, optionally substituted heteroaryl, and optionally substituted cycloalkyl;
R¹² and R¹³ are, at each instance, independently selected from H, hydroxy, and optionally substituted alkyl, or may be linked to form an optionally substituted cycloalkyl ring, or may together form ═O; and
R¹⁴ is optionally substituted alkyl,
   wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment,
A is S;
X is N;
Z is NR³_{V};
R¹ is selected from H, optionally substituted alkyl, optionally substituted cycloalkyl, optionally substituted aryl, and optionally substituted heteroaryl;
R² is selected from H, trifluoromethyl, substituted alkyl, optionally substituted alkoxy, —NR⁴R⁵, —NR⁶C(O)R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴;
R³_{I} is selected from H, halo, —CN, trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, —C≡C-J, optionally substituted cycloalkyl-J and —(NR^{a}R^{b})-J;
R³_{V} is selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, —C≡C-J, and optionally substituted cycloalkyl-J;
provided that R³_{I} is present as —C≡C-J, optionally substituted cycloalkyl-J or —(NR^{a}R^{b})-J, or R³_{V} is present as —C≡C-J or optionally substituted cycloalkyl-J;
wherein R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—;
J is selected from H and —(CR¹²R¹³)_{q}-L-M-W,
   wherein
q is 0, 1 or 2;
L is —O— or —N(G)-; and
G is selected from hydrogen, optionally substituted alkyl and optionally substituted cycloalkyl;
M is —(CR¹²R¹³)ₜ—;
t is 0, 1, 2 or 3;
W is selected from the group consisting of optionally substituted alkyl, optionally substituted alkoxy, optionally substituted alkenyl, optionally substituted cycloalkyl, optionally substituted heterocycloalkyl, optionally substituted aryl, optionally substituted heteroaryl and —NR⁸R⁹,
wherein when W is optionally substituted cycloalkyl it may optionally be bridged by a bond or optionally substituted C₁₋₂alkylene, and
wherein when W is optionally substituted heterocycloalkyl it may optionally be bridged by a bond, optionally substituted C₁₋₂alkylene, —NR⁶—, —O—, or —S(O)_{z}—; alternatively, when L=—N(G)-, L, G, M and W may be linked to form an optionally substituted heterocycloalkyl, an optionally substituted heterocycloalkenyl, or an optionally substituted heteroaryl;
z is 0, 1 or 2;
R⁴ and R⁵ are, at each instance, independently selected from H, optionally substituted alkyl, optionally substituted aryl, optionally substituted heteroaryl, and optionally substituted cycloalkyl, or are linked to form an optionally substituted heterocycloalkyl;
R⁶ is, at each instance, independently selected from H and optionally substituted alkyl;
R⁷ is, at each instance, independently selected from H and optionally substituted alkyl;
R⁸ and R⁹ are, at each instance, independently selected from H, optionally substituted alkyl, optionally substituted aryl, optionally substituted heterocycloalkyl, optionally substituted heteroaryl, and optionally substituted cycloalkyl;
R¹² and R¹³ are, at each instance, independently selected from H, hydroxy, and optionally substituted alkyl, or may be linked to form an optionally substituted cycloalkyl ring, or may together form ═O; and
R¹⁴ is optionally substituted alkyl,
   wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment,
A is S;
X is N;
Z is NR³_{V};
R¹ is selected from H, optionally substituted alkyl, optionally substituted cycloalkyl, optionally substituted aryl, and optionally substituted heteroaryl;
R² is selected from H, trifluoromethyl, substituted alkyl, optionally substituted alkoxy, —NR⁴R⁵, —NR⁶C(O)R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴;
R³_{I} is selected from trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, optionally substituted cycloalkyl-J and —(NR^{a}R^{b})-J;
R³_{V} is selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, and optionally substituted cycloalkyl-J;
provided that R³_{I} is present as optionally substituted cycloalkyl-J or —(NR^{a}R^{b})-J, or R³_{V} is present as optionally substituted cycloalkyl-J;
wherein R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—;
J is selected from H and —(CR¹²R¹³)_{q}-L-M-W,
   wherein
q is 0 or 1;
L is —O— or —N(G)-; and
G is selected from hydrogen, optionally substituted alkyl and optionally substituted cycloalkyl;
M is —(CR¹²R¹³)ₜ—;
t is 0, 1 or 2;
W is selected from the group consisting of optionally substituted alkyl, optionally substituted alkoxy, optionally substituted alkenyl, optionally substituted cycloalkyl, optionally substituted heterocycloalkyl, optionally substituted aryl, optionally substituted heteroaryl and —NR⁸R⁹,
wherein when W is optionally substituted cycloalkyl it may optionally be bridged by a bond or optionally substituted C₁₋₂alkylene, and
wherein when W is optionally substituted heterocycloalkyl it may optionally be bridged by a bond, optionally substituted C₁₋₂alkylene, —NR⁶—, —O—, or —S(O)_{z}—; alternatively, when L=—N(G)-, L, G, M and W may be linked to form an optionally substituted heterocycloalkyl, an optionally substituted heterocycloalkenyl, or an optionally substituted heteroaryl;
z is 0, 1 or 2;
R⁴ and R⁵ are, at each instance, independently selected from H, optionally substituted alkyl, optionally substituted aryl, optionally substituted heteroaryl, and optionally substituted cycloalkyl, or are linked to form an optionally substituted heterocycloalkyl;
R⁶ is, at each instance, independently selected from H and optionally substituted alkyl;
R⁷ is, at each instance, independently selected from H and optionally substituted alkyl;
R⁸ and R⁹ are, at each instance, independently selected from H, optionally substituted alkyl, optionally substituted aryl, optionally substituted heterocycloalkyl, optionally substituted heteroaryl, and optionally substituted cycloalkyl;
R¹² and R¹³ are, at each instance, independently selected from H, hydroxy, and optionally substituted alkyl, or may be linked to form an optionally substituted cycloalkyl ring, or may together form ═O; and
R¹⁴ is optionally substituted alkyl,
   wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment,
A is S;
X is N;
Z is NR³_{V};
R¹ is selected from H, optionally substituted alkyl, optionally substituted cycloalkyl, optionally substituted aryl, and optionally substituted heteroaryl;
R² is selected from H, trifluoromethyl, substituted alkyl, optionally substituted alkoxy, —NR⁴R⁵, —NR⁶C(O)R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴;
R³_{I} is —(NR^{a}R^{b})-J;
R³_{V} is selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, and optionally substituted cycloalkyl-J;
wherein R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—;
J is selected from H and —(CR¹²R¹³)_{q}-L-M-W,
   wherein
q is 0 or 1;
L is —O— or —N(G)-; and
G is selected from hydrogen, optionally substituted alkyl and optionally substituted cycloalkyl;
M is —(CR¹²R¹³)ₜ—;
t is 0, 1 or 2;
W is selected from the group consisting of optionally substituted alkyl, optionally substituted alkoxy, optionally substituted alkenyl, optionally substituted cycloalkyl, optionally substituted heterocycloalkyl, optionally substituted aryl, optionally substituted heteroaryl and —NR⁸R⁹,
wherein when W is optionally substituted cycloalkyl it may optionally be bridged by a bond or optionally substituted C₁₋₂alkylene, and
wherein when W is optionally substituted heterocycloalkyl it may optionally be bridged by a bond, optionally substituted C₁₋₂alkylene, —NR⁶—, —O—, or —S(O)_{z}—; alternatively, when L=—N(G)-, L, G, M and W may be linked to form an optionally substituted heterocycloalkyl, an optionally substituted heterocycloalkenyl, or an optionally substituted heteroaryl;
z is 0, 1 or 2;
R⁴ and R⁵ are, at each instance, independently selected from H, optionally substituted alkyl, optionally substituted aryl, optionally substituted heteroaryl, and optionally substituted cycloalkyl, or are linked to form an optionally substituted heterocycloalkyl;
R⁶ is, at each instance, independently selected from H and optionally substituted alkyl;
R⁷ is, at each instance, independently selected from H and optionally substituted alkyl;
R⁸ and R⁹ are, at each instance, independently selected from H, optionally substituted alkyl, optionally substituted aryl, optionally substituted heterocycloalkyl, optionally substituted heteroaryl, and optionally substituted cycloalkyl;
R¹² and R¹³ are, at each instance, independently selected from H, hydroxy, and optionally substituted alkyl, or may be linked to form an optionally substituted cycloalkyl ring, or may together form ═O; and
R¹⁴ is optionally substituted alkyl,
   wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment:

### A is S;

### X is N;

### Z is NR³_{V};

R¹ is selected from optionally substituted alkyl and optionally substituted phenyl;
R² is selected from H, halo, —CN, optionally substituted alkyl, —NR⁴R⁵, —NR⁶C(O)R⁷, and —CONR⁴R⁵;
R³_{I} is selected from H, —(NR^{a}R^{b})-J, optionally substituted cycloalkyl-J and —C≡C-J;
R³_{V} is selected from H, optionally substituted alkyl, —C(O)R⁷, and —SO₂R⁷;
NR^{a}R^{b} forms an optionally bridged, optionally substituted ring of formula (II):

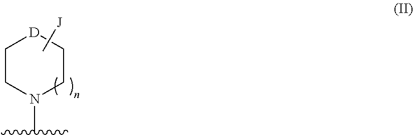

wherein n and D are defined above;
J is present in at least one instance as —(CR¹²R¹³)_{q}-L-M-W;
q is 1 or 2;
G is selected from H, optionally substituted methyl and optionally substituted ethyl;
M is selected from bond, —(CH₂)—, —(CH₂)₂—, —(CH₂)₃—, -cycloalkyl-, —CHOH—CH₂—, —CH₂—CHOH—, —CH₂—C(alkyl)₂-, —(CH₂)—C(═O)—, —C(═O)—(CH₂)—;
W is selected from substituted alkyl, alkoxy, cyclopropyl, cyclobutyl, optionally substituted pyrrolidinyl, optionally substituted piperidinyl, optionally substituted piperazinyl, optionally substituted morpholinyl, tetrahydrofuran, furan, thiophene, phenyl, and pyridine;
alternatively, when L=—N(G)-, L, G, M and W may be linked to form an optionally substituted heterocycloalkyl;
R⁴ and R⁵ are, at each instance, independently selected from H and optionally substituted alkyl, or are linked to form an optionally substituted heterocycloalkyl;

### R⁶ is H; and

R⁷ is alkyl;
wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment:

### A is S;

### X is N;

### Z is NR³_{V};

R¹ is selected from methyl, ethyl, i-propyl, and phenyl, wherein phenyl is optionally substituted by one or more of halo, —NO₂ and —SO₂N(C₁₋₆alkyl)₂;
R² is selected from H, bromo, —CN, methyl, ethyl, i-propyl, —NR⁴R⁵, —NR⁶C(O)R⁷, —CONR⁴R⁵;
R³_{I} is selected from —(NR^{a}R^{b})-J, and —C≡C-J;
R³_{V} is selected from H, alkyl, —C(O)R⁷, and —SO₂R⁷;
NR^{a}R^{b} is selected from optionally substituted pyrrolidinyl, piperidinyl, morpholinyl, piperazinyl, and 3-azabicyclo[3.1.0]hexanyl;
J is present in at least one instance as —(CR¹²R¹³)_{q}-L-M-W;
q is 1;
G is selected from H, methyl and ethyl, wherein ethyl is optionally substituted by —OH or —O—C₁₋₆alkyl;
M is selected from bond, —(CH₂)—, —(CH₂)₂—, —(CH₂)₃—, -cyclopentyl-, —CHOH—CH₂—, —CH₂—C(Me)₂-, —(CH₂)—C(═O)—;
W is selected from alkyl substituted by one or more groups selected from halo, —OH, —NH₂, and —N(C₁₋₆alkyl)₂, alkoxy, cyclopropyl, cyclobutyl, pyrrolidinyl, piperidinyl, piperazinyl, morpholinyl, tetrahydrofuran, furan, thiophene, phenyl, and pyridine, wherein each of pyrrolidinyl, piperidinyl, piperazinyl, and morpholinyl is optionally substituted by one or more groups selected from halo, C₁₋₆alkyl, —C(═O)C₁₋₆alkyl, —CO₂Cl₁₋₆alkyl, —N(C₁₋₆alkyl)₂, —NHC(═O)C₁₋₆alkyl, —C(═O)NH₂, and ═O;
alternatively, when L is —N(G)-, L, G, M and W may be linked to form azetidinyl, pyrrolidinyl, piperidinyl or morpholinyl, wherein each of pyrrolidinyl, piperidinyl and morpholinyl is optionally substituted by one or more groups selected from halo, trihalomethyl, —OH, —C₁₋₆alkyl, —O—C₁₋₆alkyl, —N(C₁₋₆alkyl)₂, —C₁₋₆alkylene-OH, aryl, haloaryl, —C(═O)NH₂ and —C₃₋₆heterocycloalkyl;
R⁴ and R⁵ are, at each instance, independently selected from H, methyl, ethyl, i-propyl, and pyrrolidinyl optionally substituted by ═O;

### R⁶ is H; and

R⁷ is methyl;
wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment:

### A is S;

### X is N;

### Z is NR³_{V};

R¹ is phenyl;

### R² is H;

R³_{I} is —(NR^{a}R^{b})-J and J is (CR¹²R¹³)_{q}-L-M-W;
R³_{V} is selected from H, methyl, —C(O)R⁷, and —SO₂R⁷;
NR^{a}R^{b} is selected from pyrrolidinyl and piperidinyl;
J is present in at least one instance as —(CR¹²R¹³)_{q}-L-M-W;
q is 1;
G is selected from H and methyl;
M is selected from bond, —(CH₂)—, —(CH₂)₂— and —(CH₂)₃—;
W is selected from pyrrolidinyl, and piperidinyl, wherein each of pyrrolidinyl and piperidinyl is optionally substituted by one of -Me, -Et and -iPr;
alternatively, when L is —N(G)-, L, G, M and W may be linked to form pyrrolidinyl, piperidinyl or morpholinyl substituted by one or more groups selected from pyrrolidinyl, —OH, —F, -Me, -OMe, —CH₂OH, —CF₃, —NMe₂, phenyl, F-phenyl, —CONH₂; and
R⁷ is methyl;
wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

In one embodiment, the compound of the invention is selected from:
4-phenyl-3-[4-(2-pyrrolidin-1-ylethoxymethyl)-1-piperidyl]-1H-thieno[2,3-c]pyrazole
5-bromo-4-phenyl-3-[5-(2-pyrrolidin-1-ylethoxy)-2-azabicyclo[2.2.1]heptan-2-yl]-1H-thieno[2,3-c]pyrazole
4-phenyl-3-[4-(2-pyrrolidin-1-ylethoxymethyl)-1-piperidyl]-1H-thieno[2,3-c]pyrazole-5-carbonitrile
4-phenyl-3-[5-(2-pyrrolidin-1-ylethoxy)-2-azabicyclo[2.2.1]heptan-2-yl]-1H-thieno[2,3-c]pyrazole
4-(4-fluorophenyl)-3-[5-(2-pyrrolidin-1-ylethoxy)-2-azabicyclo[2.2.1]heptan-2-yl]-1H-thieno[2,3-c]pyrazole
4-phenyl-3-[3-(2-pyrrolidin-1-ylethoxymethyl)-8-azabicyclo[3.2.1]octan-8-yl]-1H-thieno[2,3-c]pyrazole
4-phenyl-3-[3-(2-pyrrolidin-1-ylethoxy)-8-azabicyclo[3.2.1]octan-8-yl]-1H-thieno[2,3-c]pyrazole
4-phenyl-3-[3-(2-pyrrolidin-1-ylethoxymethyl)-1-piperidyl]-1H-thieno[2,3-c]pyrazole
4-phenyl-3-[3-(2-pyrrolidin-1-ylethoxymethyl)pyrrolidin-1-yl]-1H-thieno[2,3-c]pyrazole
N-cyclobutyl-8-(4-phenyl-1H-thieno[2,3-c]pyrazol-3-yl)-8-azabicyclo[3.2.1]octan-3-amine
3-[3-(azetidin-1-yl)-8-azabicyclo[3.2.1]octan-8-yl]-4-phenyl-1H-thieno[2,3-c]pyrazole
N-isopropyl-8-(4-phenyl-1H-thieno[2,3-c]pyrazol-3-yl)-8-azabicyclo[3.2.1]octan-3-amine
1-[4-phenyl-3-[4-(2-pyrrolidin-1-ylethoxymethyl)-1-piperidyl]thieno[2,3-c]pyrazol-1-yl]ethanone
1-[4-phenyl-3-[5-(2-pyrrolidin-1-ylethoxy)-2-azabicyclo[2.2.1]heptan-2-yl]thieno[2,3-c]pyrazol-1-yl]ethanone
1-[4-(4-fluorophenyl)-3-[5-(2-pyrrolidin-1-ylethoxy)-2-azabicyclo[2.2.1]heptan-2-yl]thieno[2,3-c]pyrazol-1-yl]ethanone
4-(4-fluorophenyl)-1-methylsulfonyl-3-[5-(2-pyrrolidin-1-ylethoxy)-2-azabicyclo[2.2.1]heptan-2-yl]thieno[2,3-c]pyrazole
1-[4-phenyl-3-[3-(2-pyrrolidin-1-ylethoxy)-8-azabicyclo[3.2.1]octan-8-yl]thieno[2,3-c]pyrazol-1-yl]ethanone
1-[4-phenyl-3-[3-(2-pyrrolidin-1-ylethoxymethyl)-1-piperidyl]thieno[2,3-c]pyrazol-1-yl]ethanone
1-[4-phenyl-3-[3-(2-pyrrolidin-1-ylethoxymethyl)pyrrolidin-1-yl]thieno[2,3-c]pyrazol-1-yl]ethanone
1-methylsulfonyl-4-phenyl-3-[4-(2-pyrrolidin-1-ylethoxymethyl)-1-piperidyl]thieno[2,3-c]pyrazole
1-methyl-4-phenyl-3-[4-(2-pyrrolidin-1-ylethoxymethyl)-1-piperidyl]thieno[2,3-c]pyrazole
N,N,1-trimethyl-4-phenyl-3-[4-(2-pyrrolidin-1-ylethoxymethyl)-1-piperidyl]thieno[2,3-c]pyrazole-5-carboxamide
N,N-dimethyl-4-phenyl-3-[4-(2-pyrrolidin-1-ylethoxymethyl)-1-piperidyl]-1H-thieno[2,3-c]pyrazole-5-carboxamide
N-isopropyl-4-phenyl-3-[4-(2-pyrrolidin-1-ylethoxymethyl)-1-piperidyl]-1H-thieno[2,3-c]pyrazole-5-carboxamide and
N-methyl-4-phenyl-3-[4-(2-pyrrolidin-1-ylethoxymethyl)-1-piperidyl]-1H-thieno[2,3-c]pyrazole-5-carboxamide.

### Chemical Groups

### Halo

The term “halogen” (or “halo”) includes fluorine, chlorine, bromine and iodine (or fluoro, chloro, bromo, and iodo).

### Alkyl, Alkylene, Alkenyl, Alkynyl, Cycloalkyl Etc.

The terms “alkyl”, “alkylene”, “alkenyl”, or “alkynyl” are used herein to refer to both straight and branched chain acyclic forms. Cyclic analogues thereof are referred to as cycloalkyl, etc.

The term “alkyl” includes monovalent, straight or branched, saturated, acyclic hydrocarbyl groups. Alkyl may be C₁₋₁₀alkyl, or C₁₋₆alkyl, or C₁₋₄alkyl. Examples include methyl, ethyl, n-propyl, i-propyl, n-butyl, s-butyl, t-butyl, n-pentyl, and s-pentyl.

The term “cycloalkyl” includes monovalent, saturated, cyclic hydrocarbyl groups. Cycloalkyl may be C₃₋₁₀cycloalkyl, or C₃₋₆cycloalkyl. Examples include cyclopropyl, cyclobutyl, cyclopentyl and cyclohexyl. A cycloalkyl may optionally be “bridged”, which occurs when ring carbon atoms are further linked by a bond, or by one or more carbon atoms. Typical bridges are one or two carbon atoms, e.g. methylene or ethylene groups. When a ring is bridged, the substituents recited for the ring may also be present on the bridge.

The term “alkoxy” means alkyl-O—. Examples include methoxy, ethoxy, n-propoxy, i-propoxy, n-butoxy, s-butoxy, t-butoxy, n-pentoxy, and s-pentoxy.

The term “alkenyl” includes monovalent, straight or branched, unsaturated, acyclic hydrocarbyl groups having at least one carbon-carbon double bond at any point along the carbon chain and, optionally, no carbon-carbon triple bonds. Alkenyl may be C₂₋₁₀alkenyl, or C₂₋₆alkenyl, or C₂₋₄alkenyl. Examples include ethenyl and propenyl.

The term “cycloalkenyl” includes monovalent, partially unsaturated, cyclic hydrocarbyl groups having at least one carbon-carbon double bond and, optionally, no carbon-carbon triple bonds. Cycloalkenyl may be C₃₋₁₀cycloalkenyl, or C₅₋₁₀cycloalkenyl. Examples include cyclohexenyl and benzocyclohexyl.

The term “alkynyl” includes monovalent, straight or branched, unsaturated, acyclic hydrocarbyl groups having at least one carbon-carbon triple bond at any point along the carbon chain and, optionally, no carbon-carbon double bonds. Alkynyl may be C₂₋₁₀alkynyl, or C₂₋₆alkynyl, or C₂₋₄alkynyl. Examples include ethynyl and propynyl.

The term “alkylene” includes divalent, straight or branched, saturated, acyclic hydrocarbyl groups. Alkylene may be C₁₋₁₀alkylene, or C₁₋₆alkylene, or C₁₋₄alkylene, such as methylene, ethylene, n-propylene, i-propylene or t-butylene groups.

The term “alkenylene” includes divalent, straight or branched, unsaturated, acyclic hydrocarbyl groups having at least one carbon-carbon double bond and, optionally, no carbon-carbon triple bonds. Alkenylene may be C₂₋₁₀alkenylene, or C₂₋₆alkenylene, or C₂₋₄alkenylene.

### Heteroalkyl Etc.

The term “heteroalkyl” includes alkyl groups in which up to three carbon atoms, or up to two carbon atoms, or one carbon atom, are each replaced independently by O, S(O)_{z} (z=0, 1 or 2) or N, provided at least one of the alkyl carbon atoms remains. The heteroalkyl group may be C-linked or hetero-linked, i.e. it may be linked to the remainder of the molecule through a carbon atom or through O, S(O)_{z} or N.

The term “heterocycloalkyl” includes cycloalkyl groups in which up to three carbon atoms, or up to two carbon atoms, or one carbon atom, are each replaced independently by O, S(O)_{z} or N, provided at least one of the cycloalkyl carbon atoms remains. Examples of heterocycloalkyl groups include oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, tetrahydro-1,3-oxazinyl, 1,4-dithianyl, piperazinyl, hexahydropyrimidinyl, 1,4-thiazanyl, oxepanyl, thiepanyl, azepanyl, 1,4-dioxepanyl, 1,4-oxathiepanyl, 1,4-oxaazepanyl, 1,4-dithiepanyl, 1,4-thieazepanyl and 1,4-diazepanyl. The heterocycloalkyl group may be C-linked or N-linked, i.e. it may be linked to the remainder of the molecule through a carbon atom or through a nitrogen atom. A heterocycloalkyl may optionally be “bridged”, which occurs when ring carbon or nitrogen atoms are further linked by a bond or one or more atoms (e.g. C, O, N, or S). Typical bridges include, but are not limited to, one carbon atom, two carbon atoms, one nitrogen atom, two nitrogen atoms, and a carbon-nitrogen group. When a ring is bridged, the substituents recited for the ring may also be present on the bridge. A cycloalkyl bridged by one or more atoms including a heteroatom (i.e. O, N, or S) may be viewed as a heterocycloalkyl with a carbon bridge. Examples of bridged heterocycloalkyl groups include azabicyclohexanyl, (e.g. 3-azabicyclo[3.1.0]hexanyl), azabicycloheptanyl (e.g. 2-azabicyclo[2.2.1]heptanyl), azabicyclooctanyl (e.g. 8-azabicyclo[3.2.1]octanyl), and 2-oxa-5-azabicyclo[2.2.1]heptane (or 5-aza-2-oxabicyclo[2.2.1]heptane). The values given herein in terms such as “4 to 7 membered heterocycloalkyl ring” refer specifically to the number of atoms present in the ring; any “bridging” atoms are counted separately.

The term “heteroalkenyl” includes alkenyl groups in which up to three carbon atoms, or up to two carbon atoms, or one carbon atom, are each replaced independently by O, S(O)_{z} or N, provided at least one of the alkenyl carbon atoms remains. The heteroalkenyl group may be C-linked or hetero-linked, i.e. it may be linked to the remainder of the molecule through a carbon atom or through O, S(O)_{z} or N.

The term “heterocycloalkenyl” includes cycloalkenyl groups in which up to three carbon atoms, or up to two carbon atoms, or one carbon atom, are each replaced independently by O, S(O)_{z} or N, provided at least one of the cycloalkenyl carbon atoms remains. Examples of heterocycloalkenyl groups include 3,4-dihydro-2H-pyranyl, 5-6-dihydro-2H-pyranyl, 2H-pyranyl, 1,2,3,4-tetrahydropyridinyl and 1,2,5,6-tetrahydropyridinyl. The heterocycloalkenyl group may be C-linked or N-linked, i.e. it may be linked to the remainder of the molecule through a carbon atom or through a nitrogen atom.

The term “heteroalkynyl” includes alkynyl groups in which up to three carbon atoms, or up to two carbon atoms, or one carbon atom, are each replaced independently by O, S(O)_{z} or N, provided at least one of the alkynyl carbon atoms remains. The heteroalkynyl group may be C-linked or hetero-linked, i.e. it may be linked to the remainder of the molecule through a carbon atom or through O, S(O)_{z} or N.

The term “heteroalkylene” includes alkylene groups in which up to three carbon atoms, or up to two carbon atoms, or one carbon atom, are each replaced independently by O, S(O)_{z} or N, provided at least one of the alkylene carbon atoms remains.

The term “heteroalkenylene” includes alkenylene groups in which up to three carbon atoms, or up to two carbon atoms, or one carbon atom, are each replaced independently by O, S(O)_{z} or N, provided at least one of the alkenylene carbon atoms remains.

The term “heterocycloalkoxy” means heterocycloalkyl-O—.

The term “heterocycloalkylalkyl” means alkyl substituted with a heterocycloalkyl group.

### Aryl

The term “aryl” includes monovalent, aromatic, cyclic hydrocarbyl groups, such as phenyl or naphthyl (e.g. 1-naphthyl or 2-naphthyl). In general, the aryl groups may be monocyclic or polycyclic fused ring aromatic groups. Preferred aryl groups are C₆-C₁₄aryl.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term “arylalkyl” means alkyl substituted with an aryl group, e.g. benzyl.

### Heteroaryl

The term “heteroaryl” includes monovalent, heteroaromatic, cyclic hydrocarbyl groups additionally containing one or more heteroatoms independently selected from O, S, N and NR^{N}, where R^{N} is selected from H, alkyl (e.g. C₁₋₆alkyl) and cycloalkyl (e.g. C₃₋₆cycloalkyl). In general, the heteroaryl groups are monocyclic or polycyclic (e.g. bicyclic) fused ring heteroaromatic groups. A heteroaryl groups may contain 5-13 ring members (preferably 5-10 members) and 1, 2, 3 or 4 ring heteroatoms independently selected from O, S, N and NR^{N}, or may be a 5, 6, 9 or 10 membered, e.g. 5-membered monocyclic, 6-membered monocyclic, 9-membered fused-ring bicyclic or 10-membered fused-ring bicyclic.

Monocyclic heteroaromatic groups include heteroaromatic groups containing 5-6 ring members and 1, 2, 3 or 4 heteroatoms selected from O, S, N or NR^{N}.

Examples of 5-membered monocyclic heteroaryl groups are pyrrolyl, furanyl, thiophenyl, pyrazolyl, imidazolyl, isoxazolyl, oxazolyl, isothiazolyl, thiazolyl, 1,2,3 triazolyl, 1,2,4 triazolyl, 1,2,3 oxadiazolyl, 1,2,4 oxadiazolyl, 1,2,5 oxadiazolyl, 1,3,4 oxadiazolyl, 1,3,4 thiadiazolyl, pyridyl, pyrimidinyl, pyridazinyl, pyrazinyl, 1,3,5 triazinyl, 1,2,4 triazinyl, 1,2,3 triazinyl and tetrazolyl.

Examples of 6-membered monocyclic heteroaryl groups are pyridinyl, pyridazinyl, pyrimidinyl and pyrazinyl.

Bicyclic heteroaromatic groups include fused-ring heteroaromatic groups containing 9-13 ring members and 1, 2, 3, 4 or more heteroatoms selected from O, S, N or NR^{N}.

Examples of 9-membered fused-ring bicyclic heteroaryl groups are benzofuranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolininyl, imidazo[1,2-a]pyridinyl, imidazo[1,5-a]pyridinyl, pyrazolo[1,2-a]pyridinyl, pyrrolo[1,2-b]pyridazinyl and imidazo[1,2-c]pyrimidinyl.

Examples of 10-membered fused-ring bicyclic heteroaryl groups are quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl and pyrimido[4,5-d]pyrimidinyl.

The term “heteroarylalkyl” means alkyl substituted with a heteroaryl group.

### General

Unless indicated explicitly otherwise, where combinations of groups are referred to herein as one moiety, e.g. arylalkyl, the last mentioned group contains the atom by which the moiety is attached to the rest of the molecule.

Where reference is made to a carbon atom of an alkyl group or other group being replaced by O, S(O)_{z} or N, what is intended is that:

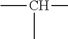

is replaced by

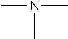

—CH═ is replaced by —N═;
≡C—H is replaced by ≡N; or
—CH₂— is replaced by —O—, —S(O)_{z}— or —NR^{N}—.

By way of clarification, in relation to the above mentioned heteroatom containing groups (such as heteroalkyl etc.), where a numerical of carbon atoms is given, for instance C₃₋₆heteroalkyl, what is intended is a group based on C₃₋₆alkyl in which one of more of the 3-6 chain carbon atoms is replaced by O, S(O)_{z} or N. Accordingly, a C₃₋₆heteroalkyl group, for example, will contain less than 3-6 chain carbon atoms.

### Substitution

Groups of the compounds of the invention (e.g. alkyl, cycloalkyl, alkoxy, alkenyl, cycloalkenyl, alkynyl, alkylene, alkenylene, heteroalkyl, heterocycloalkyl, heteroalkenyl, heterocycloalkenyl, heteroalkynyl, heteroalkylene, heteroalkenylene, heterocycloalkoxy, heterocycloalkylalkyl, aryl, arylalkyl, arylheteroalkyl, heteroaryl, heteroarylalkyl or heteroarylheteroalkyl groups etc.) may be substituted or unsubstituted. Typically, substitution involves the notional replacement of one or more hydrogen atoms on a designated atom (e.g. a carbon atom or a nitrogen atom) with one or more substituent groups (provided that the designated atom's normal valency is not exceeded), or two hydrogen atoms in the case of substitution by ═O. Alternatively, in the case of bivalent substituent groups such as C₁₋₆alkylenedioxy, substitution involves the notional replacement of a hydrogen atom on a designated atom and a hydrogen atom on an adjacent atom with the substituent group.

Where an “optionally substituted” group is indeed substituted, there will generally be 1 to 5 substituents on the group, or 1 to 3 substituents, or 1 or 2 substituents, or 1 substituent. The substituents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —C₁₋₆alkylene-NHSO₂C₁₋₆alkyl, —C₁₋₆alkylene-NHSO₂H, —C₁₋₆alkylene-NC₁₋₆alkylSO₂H, —C₁₋₆alkylene-NC₁₋₆alkylSO₂C₁₋₆alkyl, —C₁₋₆alkylene-SO₂NH₂, —C₁₋₆alkylene-SO₂NHC₁₋₆alkyl, —C₁₋₆alkylene-SO₂N(C₁₋₆alkyl)₂, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl). “C₁₋₆alkyl” and “C₁₋₆alkylene” in the above substituents may optionally be replaced by “C₁₋₆heteroalkyl” and “C₁₋₆heteroalkylene” respectively.

In one embodiment, the substituents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(═NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl). “C₁₋₆alkyl” and “C₁₋₆alkylene” in the above substituents may optionally be replaced by “C₁₋₆heteroalkyl” and “C₁₋₆heteroalkylene” respectively.

In another embodiment, the substituents are independently selected from halo, trihalomethyl, trihaloethyl, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —SO₂NH₂, —SO₂NHC₁₋₆alkyl, —SO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, ═O, —N(C₁₋₆alkyl)₂, —C(═O)NH₂, —C(═O)NHC₁₋₆alkyl, —C(═O)N(C₁₋₆alkyl)₂, —C₁₋₆alkyl, —C₃₋₆cycloalkyl, —C₃₋₆heterocycloalkyl, aryl, haloaryl, —Z^{t}H, —Z^{t}—C₁₋₆alkyl, —C₁₋₆alkylene-Z^{t}H or —Z^{t}—C₃₋₆cycloalkyl, wherein Z^{t} is independently O, S, NH or N(C₁₋₆alkyl).

Where a group has at least 2 positions which may be substituted, the group may be substituted by both ends of an alkylene or heteroalkylene chain to form a cyclic moiety.

The molecular weight of the compounds of the invention may, optionally, be less than 1000 g/mole, or less than 950 g/mole, or less than 900 g/mole, or less than 850 g/mole, or less than 800 g/mole, or less than 750 g/mole, or less than 700 g/mole, or less than 650 g/mole, or less than 600 g/mole, or less than 550 g/mole, or less than 500 g/mole.

The compounds of the invention may include any isotopes of the atoms comprised in the compounds. Examples include ²H and ³H, and ¹³C and ¹⁴C.

### Pharmaceutically Acceptable Derivatives

The term “pharmaceutically acceptable derivative” includes any pharmaceutically acceptable salt, solvate, hydrate or prodrug of a compound of the invention. In one embodiment, the pharmaceutically acceptable derivatives are pharmaceutically acceptable salts, solvates or hydrates of a compound of the invention.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Pharmaceutically Acceptable Salts

The term “pharmaceutically acceptable salt” includes a derivative of a compound of the invention that is a salt prepared from pharmaceutically acceptable non-toxic acids or bases including inorganic or organic acids and bases.

Compounds of the invention which contain basic, e.g. amino, groups are capable of forming pharmaceutically acceptable salts with acids. Pharmaceutically acceptable acid addition salts of the compounds of the invention may include, but are not limited to, those of inorganic acids such as hydrohalic acids (e.g. hydrochloric, hydrobromic and hydroiodic acid), sulfuric acid, sulfamic acid, nitric acid, and phosphoric acid. Pharmaceutically acceptable acid addition salts of the compounds of the invention may include, but are not limited to, those of organic acids such as aliphatic, aromatic, carboxylic and sulfonic classes of organic acids, examples of which include: aliphatic monocarboxylic acids such as formic acid, acetic acid, propionic acid or butyric acid; aliphatic hydroxy acids such as lactic acid, citric acid, tartaric acid or malic acid; dicarboxylic acids such as oxalic acid, maleic acid, hydroxymaleic acid, fumaric acid or succinic acid; aromatic carboxylic acids such as benzoic acid, p-chlorobenzoic acid, 2-acetoxybenzoic acid, phenylacetic acid, diphenylacetic acid or triphenylacetic acid; aromatic hydroxyl acids such as o-hydroxybenzoic acid, p-hydroxybenzoic acid, 1-hydroxynaphthalene-2-carboxylic acid or 3 hydroxynaphthalene-2-carboxylic acid; and sulfonic acids such as methanesulfonic acid, ethanesulfonic acid, ethanedisulfonic acid, isethionic acid, benzenesulfonic acid, toluenesulfonic acid. Other pharmaceutically acceptable acid addition salts of the compounds of the invention include, but are not limited to, those of ascorbic acid, glycolic acid, glucuronic acid, furoic acid, glutamic acid, anthranilic acid, salicylic acid, mandelic acid, embonic (pamoic) acid, pantothenic acid, stearic acid, sulfanilic acid, algenic acid, and galacturonic acid. Compounds of the invention which contain acidic, e.g. carboxyl, groups are capable of forming pharmaceutically acceptable salts with bases. In one embodiment, pharmaceutically acceptable basic salts of the compounds of the invention include, but are not limited to, metal salts such as alkali metal or alkaline earth metal salts (e.g. sodium, potassium, magnesium or calcium salts) and zinc or aluminium salts. In one embodiment, pharmaceutically acceptable basic salts of the compounds of the invention include, but are not limited to, salts formed with ammonia or pharmaceutically acceptable organic amines or heterocyclic bases such as ethanolamines (e.g. diethanolamine), benzylamines, N-methyl-glucamine, amino acids (e.g. lysine) or pyridine.

Hemisalts of acids and bases may also be formed, e.g. hemisulphate salts.

Pharmaceutically acceptable salts of compounds of the invention may be prepared by methods well-known in the art. For instance, such salts can be prepared by reacting the free acid or base forms of these compounds with a stoichiometric amount of the appropriate base or acid in water or in an organic solvent, or in a mixture of the two. Generally, nonaqueous media like ether, ethyl acetate, ethanol, isopropanol, or acetonitrile are preferred. For a review of pharmaceutically acceptable salts, see Stahl and Wermuth, Handbook of Pharmaceutical Salts: Properties, Selection and Use (Wiley-VCH, Weinheim, Germany, 2002).

### Solvates & Hydrates

The compounds of the invention may exist in both unsolvated and solvated forms. The term “solvate” includes molecular complexes (e.g. crystals) comprising a compound of the invention and one or more pharmaceutically acceptable solvent molecules such as water or C₁₋₆ alcohols, e.g. ethanol. The term “hydrate” means a “solvate” where the solvent is water.

### Prodrugs

The invention includes prodrugs of the compounds of the invention. Prodrugs are derivatives of compounds of the invention (which may have little or no pharmacological activity themselves), which can, when administered in vivo, be converted into compounds of the invention.

Prodrugs can, for example, be produced by replacing functionalities present in the compounds of the invention with appropriate moieties which are metabolized in vivo to form a compound of the invention. The design of prodrugs is well-known in the art, as discussed in Bundgaard, Design of Prodrugs 1985 (Elsevier), The Practice of Medicinal Chemistry 2003, 2nd Ed, 561-585 and Leinweber, Drug Metab. Res. 1987, 18: 379.

Examples of prodrugs of compounds of the invention are esters and amides of the compounds of the invention. For example, where the compound of the invention contains a carboxylic acid group (—COOH), the hydrogen atom of the carboxylic acid group may be replaced to form an ester (e.g. the hydrogen atom may be replaced by —C₁₋₆alkyl). Where the compound of the invention contains an alcohol group (—OH), the hydrogen atom of the alcohol group may be replaced in order to form an ester (e.g. the hydrogen atom may be replaced by —C(O)C₁₋₆alkyl. Where the compound of the invention contains a primary or secondary amino group, one or more hydrogen atoms of the amino group may be replaced in order to form an amide (e.g. one or more hydrogen atoms may be replaced by —C(O)C₁₋₆alkyl).

### Amorphous & Crystalline Forms

The compounds of the invention may exist in solid states from amorphous through to crystalline forms. “Amorphous” refers to a solid form of a molecule, atom, and/or ions that is not crystalline. Different crystalline forms (“polymorphs”) have the same chemical composition but different spatial arrangements of the molecules, atoms, and/or ions forming the crystal. All such solid forms are included within the invention.

### Purity

The compounds of the invention may, subsequent to their preparation, be isolated and purified to obtain a composition containing an amount by weight equal to or greater than 99% of said compound (“substantially pure” compound), which is then used or formulated as described herein.

### Isomeric Forms

Compounds of the invention may exist in one or more geometrical, optical, enantiomeric, diastereomeric and tautomeric forms, including but not limited to cis- and trans-forms, E- and Z-forms, R-, S- and meso-forms, keto-, and enol-forms. All such isomeric forms are included within the invention. The isomeric forms may be in isomerically pure or enriched form (e.g. one enantiomer may be present in excess, also known as a scalemic mixture), as well as in mixtures of isomers (e.g. racemic or diastereomeric mixtures).

If one enantiomer is present in a greater amount that its corresponding enantiomer, the enantiomeric excess may be expressed as a percentage of the whole. For instance, a 98:2 mixture of one enantiomer to another has a 96% enantiomeric excess of the first enantiomer. The enantiomeric excess may be at least 5%, 10%, 15%, 20%, 25%, 30%, 35%, 40%, 45%, 50%, 55%, 60%, 65%, 70%, 75%, 80%, 85%, 90%, 95% or up to 100% (i.e. enantiomerically pure, up to the detection limit of purity).

The invention therefore provides:
stereoisomeric mixtures of compounds of the invention;
a diastereomerically enriched or diastereomerically pure isomer of a compound of the invention; or
an enantiomerically enriched or enantiomerically pure isomer of a compound of the invention.

The processes for preparation can utilize racemates, enantiomers, or diastereomers as starting materials. Where appropriate, isomers can be prepared by the application or adaptation of known methods (e.g. asymmetric synthesis). When diastereomeric or enantiomeric products are prepared, they can be separated by conventional methods for example, chromatographic or fractional crystallization.

### Isotopic Labeling

The invention includes pharmaceutically acceptable isotopically-labelled compounds of the invention wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of the invention, for example, those incorporating a radioactive isotope, are useful in drug and/or substrate tissue distribution studies. The radioactive isotopes ³H and ¹⁴C are particularly useful for this purpose in view of their ease of incorporation and ready means of detection. Substitution with heavier isotopes such as ²H may afford certain therapeutic advantages resulting from greater metabolic stability, for example, increase in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of the invention can generally be prepared by conventional techniques known to those skilled in the art or by processes analogous to those described herein using an appropriate isotopically-labelled reagent in place of the non-labelled reagent previously employed.

### Treatment of Diseases and Conditions

Compounds of the invention are inhibitors of Kᵢᵣ3.1 and/or Kᵢᵣ3.4.

The invention provides a compound of the invention for use in therapy. The invention further provides a pharmaceutical composition comprising a compound of the invention in combination with a pharmaceutically acceptable excipient.

The invention further provides a method for the treatment of a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, comprising the step of administering a therapeutically effective amount of a compound of the invention to a patient. The invention also provides the use of a compound of the invention for the manufacture of a medicament for the treatment of a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof. The invention also provides a compound of the invention for use in a method for the treatment of a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof.

Preferred compounds of the invention have an IC₅₀ in the Kir3.1/3.4 Electrophysiology Method (described below) of <100 μM, <10 μM, <3 μM, <1 μM, <100 nM, or <10 nM.

Diseases and Conditions Mediated by Kᵢᵣ3.1 and/or Kᵢᵣ3.4 or Heteromultimers Thereof/Requiring Inhibition of Kᵢᵣ3.1 and/or Kᵢᵣ3.4 or Heteromultimers Thereof

The invention is useful for the treatment of a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof. In particular, the heteromultimer may be the heterotetramer Kᵢᵣ3.1/3.4. The invention therefore has use in:
the treatment of cardiovascular diseases, such as atrial fibrillation (AF), atrial flutter (AFL), atrioventricular (AV) dysfunction and sinoatrial node (SAN) dysfunction;
the prevention of recurrence of supraventriclar arrhythmias including AF and AFL;
the maintenance of sinus rhythm;
the termination and cardioversion of supraventriclar arrhythmias;
the treatment of sinus node dysfunction;
the treatment of AV node dysfunction, including AV block;
the treatment of conduction dysfunction;
the prevention or reversal of atrial structural and ionic remodeling;
the prevention of thrombosis, thromboembolism and thromboembolic diseases, such as stroke, myocardial infarction, and peripheral vascular diseases;
the improvement of cardiac contractility;
the treatment of metabolic diseases, such as diabetes mellitus;
the modulation of neuro-endocrine function;
the modulation of the secretion of pituitary hormones;
the treatment of neurological and neuropsychiatric disorders, such as pain, depression, anxiety, attention deficit/hyperactivity disorder and epilepsy; and
the treatment of cancer, such as breast cancer.

### Therapeutic Definitions

As used herein, “treatment” includes curative, modulative (i.e. arresting the development of a disease state) and prophylactic treatment. As used herein, a “patient” means an animal, such as a mammal, such as a human, in need of treatment.

The amount of the compound of the invention administered should be a therapeutically effective amount where the compound or derivative is used for the treatment of a disease or condition, or its modulation, and a prophylactically effective amount where the compound or derivative is used for the prevention of a disease or condition.

The term “therapeutically effective amount” used herein refers to the amount of compound needed to treat or ameliorate a targeted disease or condition. The term “prophylactically effective amount” used herein refers to the amount of compound needed to prevent a targeted disease or condition. The exact dosage will generally be dependent on the patient's status at the time of administration. Factors that may be taken into consideration when determining dosage include the severity of the disease state in the patient, the general health of the patient, the age, weight, gender, diet, time, frequency and route of administration, drug combinations, reaction sensitivities and the patient's tolerance or response to therapy. The precise amount can be determined by routine experimentation, but may ultimately lie with the judgement of the clinician. Generally, an effective dose will be from 0.01 mg/kg/day (mass of drug compared to mass of patient) to 1000 mg/kg/day, e.g. 1 mg/kg/day to 100 mg/kg/day.

Compounds of the invention may be administered in a single daily dose, or the total daily dosage may be administered in divided doses of two, three, or four times daily. The daily oral dosage of the active ingredient may be between 3 and 600 mg either administered once daily or in divided doses administered twice daily. Alternatively, the active ingredient may be administered in doses of 10-20 mg administered twice daily or 40 to 100 mg administered once daily. Alternatively, the active ingredient may be administered a dose of 12.5 mg twice a day or 75 mg once a day. Alternatively, the active ingredient may be administered in doses of 3, 10, 30, 100, 300, and 600 mg administered either once or twice a day. Compositions may be administered individually to a patient or may be administered in combination with other agents, drugs or hormones.

### Administration & Formulation

### General

For pharmaceutical use, the compounds of the invention may be administered as a medicament by enteral or parenteral routes, including intravenous, intramuscular, subcutaneous, transdermal, airway (aerosol), oral, intranasal, rectal, vaginal and topical (including buccal and sublingual) administration. The compounds of the invention should be assessed for their biopharmaceutical properties, such as solubility and solution stability (across pH), permeability, etc., in order to select the most appropriate dosage form and route of administration for treatment of the proposed indication.

The compounds of the invention may be administered as crystalline or amorphous products. The compounds of the invention may be administered alone or in combination with one or more other compounds of the invention or in combination with one or more other drugs (or as any combination thereof). Generally, they will be administered as a formulation in association with one or more pharmaceutically acceptable excipients. The term “excipient” includes any ingredient other than the compound(s) of the invention which may impart either a functional (e.g. drug release rate controlling) and/or a non-functional (e.g. processing aid or diluent) characteristic to the formulations. The choice of excipient will to a large extent depend on factors such as the particular mode of administration, the effect of the excipient on solubility and stability, and the nature of the dosage form.

Typical pharmaceutically acceptable excipients include:
diluents, e.g. lactose, dextrose, sucrose, mannitol, sorbitol, cellulose and/or glycine;
lubricants, e.g. silica, talcum, stearic acid, its magnesium or calcium salt and/or polyethyleneglycol;
binders, e.g. magnesium aluminum silicate, starch paste, gelatin, tragacanth, methylcellulose, sodium carboxymethylcellulose and/or polyvinylpyrrolidone;
disintegrants, e.g. starches, agar, alginic acid or its sodium salt, or effervescent mixtures; and/or
absorbants, colorants, flavors and/or sweeteners.

A thorough discussion of pharmaceutically acceptable excipients is available in Gennaro, Remington: The Science and Practice of Pharmacy 2000, 20th edition (ISBN: 0683306472).

Accordingly, the present invention provides a pharmaceutical composition comprising a compound of the invention and a pharmaceutically acceptable excipient.

Compounds of the invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) may contain from about 1 milligram to about 500 milligrams of active ingredient per dosage unit. In these pharmaceutical compositions the active ingredient will typically be present in an amount of about 0.5-95% by weight based on the total weight of the composition.

### Oral Administration

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions, or solutions in a digestible oil, such as soybean oil, cottonseed oil or olive oil), emulsions or powders; lozenges (including liquid-filled); chews; gels; fast dispersing dosage forms; powders; granules; films; ovules; sprays; and buccal/mucoadhesive patches.

Formulations suitable for oral administration may also be designed to deliver the compounds of the invention in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups, tinctures and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents 2001, 11(6): 981-986.

The formulation of tablets is discussed in H. Lieberman and L. Lachman, Pharmaceutical Dosage Forms: Tablets 1980, vol. 1 (Marcel Dekker, New York).

### Parenteral Administration

The compounds of the invention can be administered parenterally.

The compounds of the invention may be administered directly into the blood stream, into subcutaneous tissue, into muscle, or into an internal organ. Suitable means for administration include intravenous, intraarterial, intraperitoneal, intrathecal, intraventricular, intraurethral, intrasternal, intracranial, intramuscular, intrasynovial and subcutaneous. Suitable devices for administration include needle (including microneedle) injectors, needle-free injectors and infusion techniques.

Parenteral formulations are typically aqueous or oily solutions and may include, as carriers, water, a suitable oil, saline, aqueous dextrose (glucose) and related sugar solutions, and/or glycols such as propylene glycol or polyethylene glycols. Where the solution is aqueous, excipients such as sugars (including but restricted to glucose, mannitol, sorbitol, etc.) salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water (WFI).

Solutions for parenteral administration may contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propylparaben, and chlorobutanol.

Parenteral formulations may include implants derived from degradable polymers such as polyesters (i.e. polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

### Inhalation & Intranasal Administration

The compounds of the invention can be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as 1 leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

Formulations for inhaled/intranasal administration may be formulated to be immediate and/or modified release using, for example, PGLA. Modified release formulations include delayed-, sustained-, pulsed-, controlled-, targeted and programmed release.

### Transdermal Administration

Suitable formulations for transdermal application include a therapeutically effective amount of a compound of the invention with carrier. Advantageous carriers include absorbable pharmacologically acceptable solvents to assist passage through the skin of the host.

Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

### Combination Therapy

A compound of the invention may be administered alone, or may be administered in combination with another therapeutic agent (i.e. a different agent to the compound of the invention). The compound of the invention and the other therapeutic agent may be administered in a therapeutically effective amount.

The compound of the present invention may be administered either simultaneously with, or before or after, the other therapeutic agent. The compound of the present invention and the other therapeutic agent may be administered separately, by the same or different route of administration, or together in the same pharmaceutical composition.

In one embodiment, the invention provides a product comprising a compound of the invention and another therapeutic agent as a combined preparation for simultaneous, separate or sequential use in therapy. In one embodiment, the therapy is the treatment of a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof. Products provided as a combined preparation include a composition comprising the compound of the invention and the other therapeutic agent together in the same pharmaceutical composition, or the compound of the invention and the other therapeutic agent in separate form, e.g. in the form of a kit.

The invention provides a pharmaceutical composition comprising a compound of the invention and another therapeutic agent. Optionally, the pharmaceutical composition may comprise a pharmaceutically acceptable excipient, as described above in “Administration & Formulation”.

The invention provides a kit comprising two or more separate pharmaceutical compositions, at least one of which contains a compound the invention. The kit may comprise means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

The compound of the invention and the other therapeutic agent may be combined in a single dosage unit. Optionally, they may be formulated such that although the active ingredients are combined in a single dosage unit, the physical contact between the active ingredients is minimized. For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material which effects a sustained-release throughout the gastrointestinal tract and also serves to minimize physical contact between the combined active ingredients. Furthermore, the sustained-released component can be additionally enteric coated such that the release of this component occurs only in the intestine. Still another approach can involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

Accordingly, the invention provides the use of a compound of the invention in the manufacture of a medicament for treating a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, wherein the medicament is prepared for administration with another therapeutic agent. The invention also provides the use of another therapeutic agent in the manufacture of medicament for treating a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, wherein the medicament is prepared for administration with a compound of the invention.

The invention also provides a compound of the invention for use in a method of treating a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, wherein the compound of the invention is prepared for administration with another therapeutic agent. The invention also provides another therapeutic agent for use in a method of treating a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, wherein the other therapeutic agent is prepared for administration with a compound of the invention. The invention also provides a compound of the invention for use in a method of treating a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, wherein the compound of the invention is administered with another therapeutic agent. The invention also provides another therapeutic agent for use in a method of treating a disease or condition mediated by Kir3.1 and/or Kir3.4 or any heteromultimers thereof, or that requires inhibition of Kᵢᵣ3.1 and/or Kᵢᵣ3.4 or any heteromultimers thereof, wherein the other therapeutic agent is administered with a compound of the invention.

The invention also provides the use of a compound of the invention in the manufacture of a medicament for treating a disease or condition mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, wherein the patient has previously (e.g. within 24 hours) been treated with another therapeutic agent. The invention also provides the use of another therapeutic agent in the manufacture of a medicament for treating a disease or condition mediated by Kir3.1 and/or Kir3.4 or any heteromultimers thereof, or that requires inhibition of Kᵢᵣ3.1 and/or Kᵢᵣ3.4 or any heteromultimers thereof, wherein the patient has previously (e.g. within 24 hours) been treated with a compound of the invention.

In one embodiment, the other therapeutic agent is selected from other antiarrythmic agents, such as Vaughan-Williams class I, class II, class III, or class IV agents, or from other cardiovascular agents.

### Synthesis

Compounds of formula (I) may be prepared by conventional routes, for example those set out in Schemes 1 to 5 shown below.

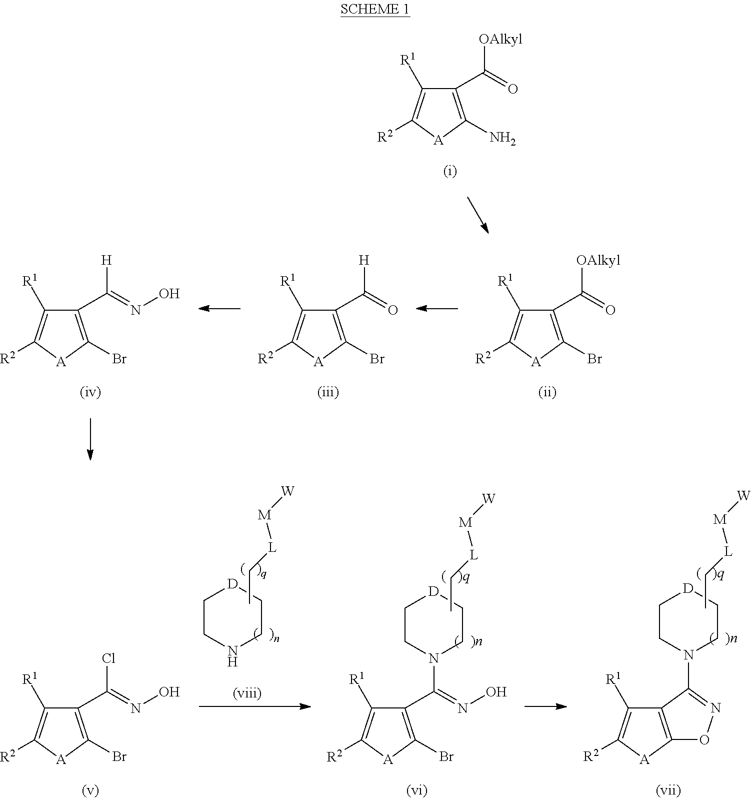

Compounds of formula (vii) may be prepared as shown in scheme 1 from compounds of formula (vi) via a cyclisation in the presence of base such as potassium carbonate. Compounds of formula (vi) may be prepared via reaction of compounds of formula (v) with compounds of formula (viii). Compounds of formula (v) may be prepared from compounds of formula (iv) by chlorination with a suitable reagent such as N-Chlorosuccinimide or oxone/HCl. Compounds of formula (iv) may be prepared from compounds of formula (iii) via reaction with hydroxylamine or hydroxylamine hydrochloride. Compounds of formula (iii) may be prepared from compounds of formula (ii) via reduction using a suitable reducing agent such as diisobutyllithium aluminium hydride. Alternatively, the reaction may be accomplished in two stages with full reduction of the alkyl ester to the alcohol using a suitable reducing agent such as lithium borohydride followed by oxidation to the aldehyde using a suitable oxidising agent such as manganese dioxide or pyridinium chlorochromate. Compounds of formula (ii) may be prepared from compounds of formula (i) via a Sandmeyer-type reaction using a suitable diazotizing agent such as t-butylnitrite and copper (II) bromide. Compounds of formula (i) are known compounds or may be prepared by standard published methods familiar to those skilled in the art.

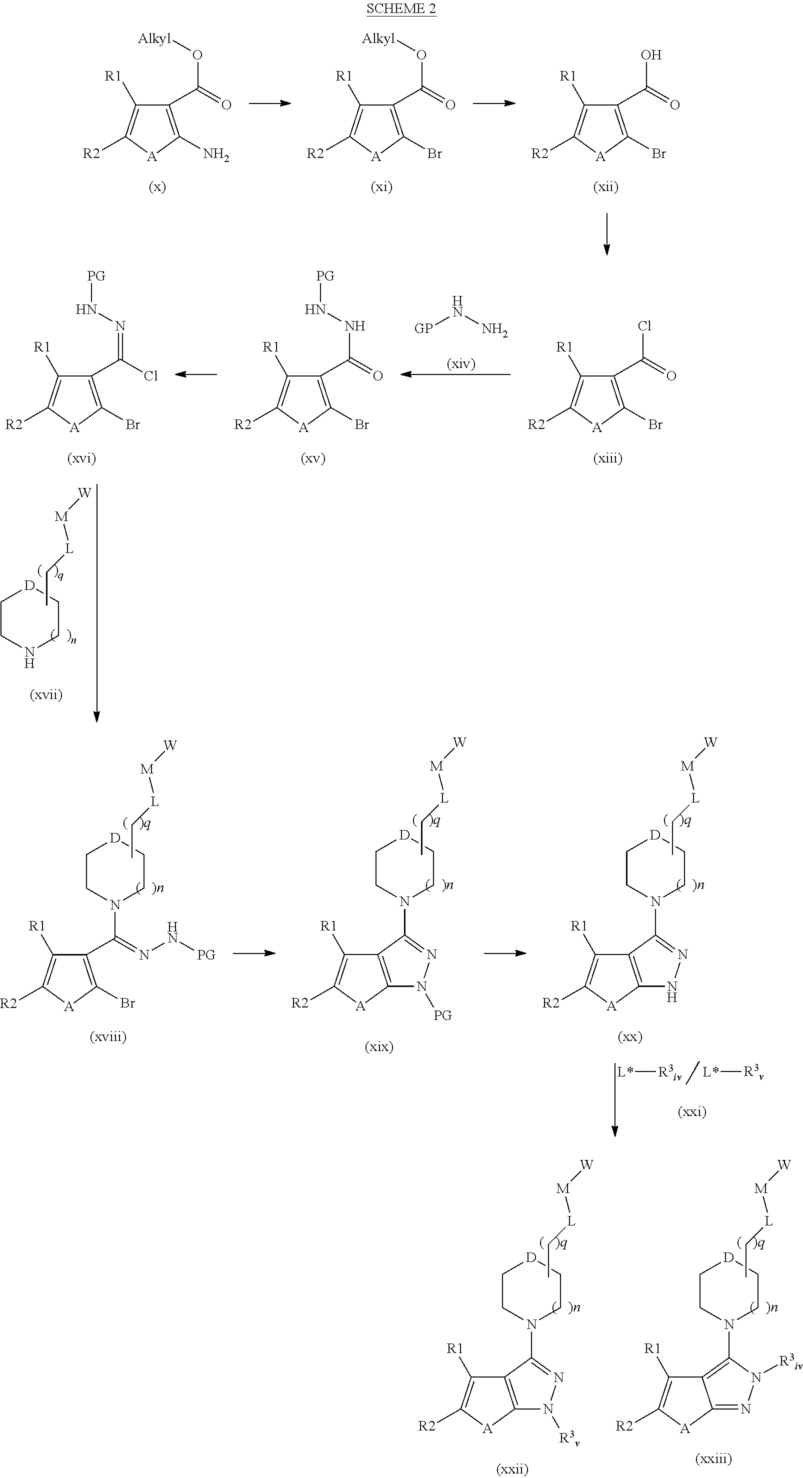

Compounds of formula (xxii) or (xxiii) may be prepared as shown in scheme 2 from compounds of formula (xx) via attack of an electrophile (xxi) (L*-R³_{IV}/L*-R³_{V}, where L* is a suitable leaving group) on the nitrogen. Compounds of formula (xx) may be prepared by removal of a suitable protecting group (PG). Suitable protecting groups include toluenesulfonyl. Compounds of formula (xix) may be prepared by a cyclisation of compounds of formula (xviii) in the presence of base such as potassium carbonate. Compounds of formula (xviii) may be prepared via reaction of compounds of formula (xvi) with compounds of formula (xvii). Compounds of formula (xvii) are known compounds or may be prepared by standard published methods familiar to those skilled in the art, or may be prepared as shown in Scheme 5. Compounds of formula (xvi) may be prepared by chlorination of compounds of formula (xv) with a chlorinating agent such as thionyl chloride. Compounds of formula (xv) may be prepared via reaction of compounds of formula (xiii) with hydrazines of formula (xiv). Compounds of formula (xiv) are known compounds or may be prepared by standard published methods familiar to those skilled in the art. Compounds of formula (xiii) may be prepared from compounds of formula (xii) by chlorination with a suitable reagent such as thionyl chloride or oxalyl chloride. Compounds of formula (xii) may be prepared from compounds of formula (xi) using standard methods familiar to those skilled in the art. Alternatively they may be commercially available. Compounds of formula (xi) may be prepared from compounds of formula (x) via a Sandmeyer-type reaction using a suitable diazotizing agent such as t-butylnitrite and copper (II) bromide. Alternatively they may be commercially available. Compounds of formula (x) are known compounds or may be prepared by standard published methods familiar to those skilled in the art. Alternatively they may be commercially available.

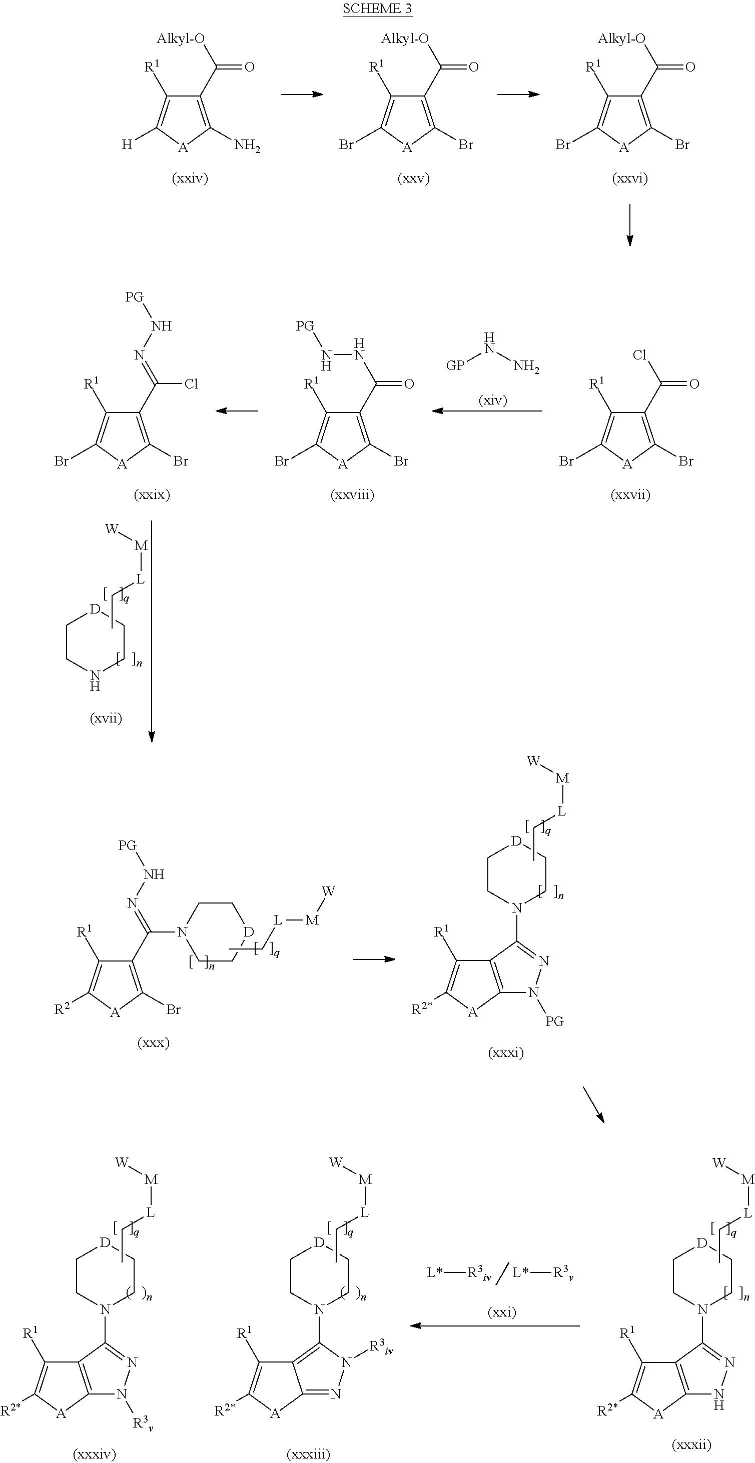

Compounds of formula (xxxiii) or (xxxiv) may be prepared as shown in Scheme 3 from compounds of formula (xxxii) via attack of an electrophile (xxi) (L*-R³_{IV}/L*-R³_{V}, where L* is a suitable leaving group such as chloro) on the nitrogen. Compounds of formula (xxxii) may be prepared by removal of a suitable protecting group (PG) from compounds of formula (xxxi). Suitable protecting groups include toluenesulfonyl. Compounds of formula (xxxi) may be prepared by a cyclisation of compounds of formula (xxx) in the presence of a basic mixture such as potassium carbonate and copper (I) iodide. In this step Br may be replaced by R²* (where R²*=H) under the reaction conditions. Alternatively Br may be replaced by R²* in any of the subsequent steps. Example reactions include cyanation with zinc cyanide catalysed by palladium, hydrogenation and no reaction. R²* may be further transformed in subsequent steps, for example hydrolysis of nitrile to carboxylic acid or ester followed by amide formation or decarboxylation. Compounds of formula (xxx) may be prepared via reaction of compounds of formula (xxix) with compounds of formula (xvii). Compounds of formula (xvii) are known compounds or may be prepared by standard published methods familiar to those skilled in the art, or may be prepared as shown in Scheme 5. Compounds of formula (xxix) may be prepared by chlorination of compounds of formula (xxviii) with a chlorinating agent such as thionyl chloride. Compounds of formula (xxviii) may be prepared via reaction of compounds of formula (xxvii) with hydrazines of formula (xiv). Compounds of formula (xiv) are known compounds or may be prepared by standard published methods familiar to those skilled in the art. Compounds of formula (xxvii) may be prepared from compounds of formula (xxvi) by chlorination with a suitable reagent such as thionyl chloride or oxalyl chloride. Compounds of formula (xxvi) may be prepared from compounds of formula (xxv) using standard methods familiar to those skilled in the art. Alternatively they may be commercially available. Compounds of formula (xxv) may be prepared from compounds of formula (xxiv) via a Sandmeyer-type reaction using a suitable diazotizing agent such as t-butylnitrite and copper (II) bromide. Alternatively they may be commercially available. Compounds of formula (xxiv) are known compounds or may be prepared by standard published methods familiar to those skilled in the art. Alternatively they may be commercially available.

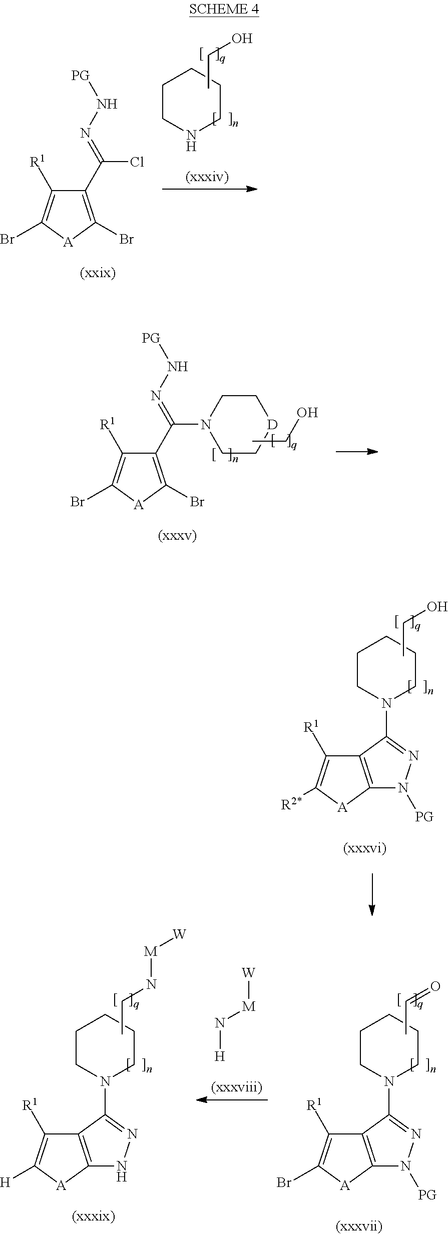

Compounds of formula (xxxix) may be prepared as shown in Scheme 4 from compounds of formula (xxxvii) and primary or secondary amines of formula (xxxviii) by reductive amination catalysed by dibutyltin dichloride followed by treatment with base. Compounds of formula (xxxvii) may be prepared from compounds of formula (xxxvi) by oxidation with a suitable oxidising agent such as Dess Martin Periodinane. Compounds of formula (xxxvi) may be prepared by a cyclisation of compounds of formula (xxxv) in the presence of a basic mixture such as potassium carbonate and copper (I) iodide. Compounds of formula (xxxv) may be prepared by reaction of compounds of formula (xxix) with compounds of formula (xxxiv). Compounds of formula (xxxiv) are known or may be commercially available. Compounds of formula (xxix) may be prepared according to Scheme 3.

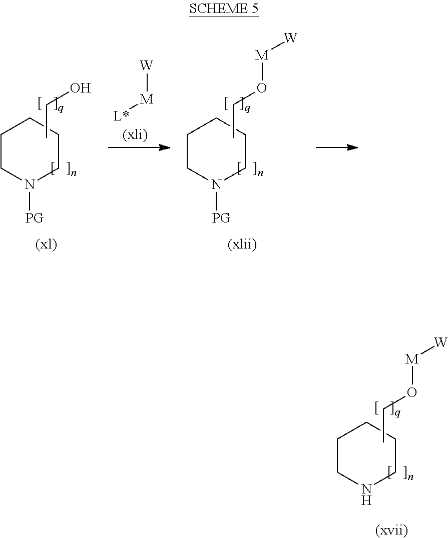

Compounds of formula (xvii) may be prepared as shown in Scheme 5 from compounds of formula (xlii) by removal of a suitable protecting group (PG) using standard methods. Suitable protecting groups include tertbutoxycarbonyl and benzyloxycarbonyl. Compounds of formula (xlii) may be prepared by reaction of compounds of formula (xl) with alkylating agents of formula (xli) (where L* is a suitable leaving group). Compounds of formula (xli) are commercially available. Compounds of formula (xl) are commercially available or may be prepared by standard methods.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

### MODES FOR CARRYING OUT THE INVENTION

### Experimental Section

Many of the starting materials referred to in the reactions described below are available from commercial sources or can be made by methods cited in the literature references.

### Analytical Methods

HPLC analysis was conducted using the following methods:

AGILENT 6110/1200 LCMS system

Solvent: [H₂O-0.1% HCO₂H:MeCN-0.05% HCO₂H:H₂O-0.1% HCO₂H], 10-95% gradient 3 min, 95% 3-5 min, 5.5-5.8 min 95%-20% gradient; 6 min 5%; Column: Phenomenex Gemini 50×4.6 mm i.d., 3 micronC18 reverse phase; Flow rate: 0.75 mL/min. UV detection 220/254 nm, MS Electrospray (+ve and −ve mode).

Preparative HPLC purification was conducted in the following manner:

Solvent: [MeCN-0.05% HCO₂H: H₂O-0.1% HCO₂H], 5-95% gradient 12 min, 95% 3 min; Waters X-Bridge 100×19 mm i.d., C18 reverse phase; Flow rate: 16 mL/min unless otherwise indicated.

HPLC: Agilent HPLC with Waters XBridge C18, 5 μm, 100 mm×19 mm i.d. column and a flow rate of 16 ml/minute. With two G1361A prep pumps, a G2258A duel loop auto sampler, a G1315 diode array detector and a G3064B prep fraction collector. Analysed by ChemStation 3. Solvents, (acidic method) water with 0.01% formic acid and acetonitrile with 0.05% formic acid or (basic method) water with 0.1% ammonia and acetonitrile.

Method a:

Time minutes

| | 0 | 12 | 15 | 15.5 |
|---|---|---|---|---|
| Acetonitrile concentration % | 5 | 95 | 95 | 5 |

Method b:

| | Time minutes | | | | | | |
|---|---|---|---|---|---|---|---|
| | 0 | 1.5 | 13.5 | 14 | 15 | 15.5 | 16 |
| Acetonitrile concentration % | 5 | 40 | 65 | 98 | 98 | 5 | 5 |

Method c:

| | Time minutes | | | | | |
|---|---|---|---|---|---|---|
| | 0 | 1.5 | 14 | 14.5 | 15 | 15.5 |
| Acetonitrile concentration % | 5 | 50 | 75 | 95 | 95 | 5 |

Method d:

| | Time minutes | | | | | |
|---|---|---|---|---|---|---|
| | 0 | 1.5 | 14 | 14.5 | 15 | 15.5 |
| Acetonitrile concentration % | 5 | 50 | 65 | 95 | 95 | 5 |

Method e:

| | Time minutes | | | | |
|---|---|---|---|---|---|
| | 0 | 11 | 12 | 15 | 15 |
| Acetonitrile concentration % | 5 | 35 | 95 | 95 | 5 |

Method f:

| | Time minutes | | | | |
|---|---|---|---|---|---|
| | 0 | 1.5 | 12 | 15 | 15.5 |
| Acetonitrile concentration % | 5 | 50 | 95 | 95 | 5 |

Method g:

| | Time minutes | | | | |
|---|---|---|---|---|---|
| | 0 | 11 | 11.5 | 14.5 | 15 |
| Acetonitrile concentration % | 5 | 55 | 95 | 95 | 5 |

HPLC: Agilent HPLC with Phenomenex Gemini-NX, 5 μm, 100 mm×30 mm i.d. column and a flow rate of 40 ml/minute. With two G1361A prep pumps, a G2258A duel loop auto sampler, a G1315 diode array detector and a G3064B prep fraction collector. Analysed by ChemStation 3. Solvents, (acidic method) water with 0.01% formic acid and acetonitrile with 0.05% formic acid or (basic method) water with 0.1% ammonia and acetonitrile.

Method 1:

Time minutes

| | 0 | 12 | 15 | 15.5 |
|---|---|---|---|---|
| Acetonitrile concentration % | 5 | 95 | 95 | 5 |

Method 2:

| | Time minutes | | | | | | |
|---|---|---|---|---|---|---|---|
| | 0 | 1.5 | 13.5 | 14 | 15 | 15.5 | 16 |
| Acetonitrile concentration % | 5 | 40 | 65 | 98 | 98 | 5 | 5 |

Method 3:

| | Time minutes | | | | | |
|---|---|---|---|---|---|---|
| | 0 | 1.5 | 14 | 14.5 | 15 | 15.5 |
| Acetonitrile concentration % | 5 | 50 | 75 | 95 | 95 | 5 |

Method 4:

| | Time minutes | | | | | |
|---|---|---|---|---|---|---|
| | 0 | 1.5 | 14 | 14.5 | 15 | 15.5 |
| Acetonitrile concentration % | 5 | 50 | 65 | 95 | 95 | 5 |

Method 5:

| | Time minutes | | | | |
|---|---|---|---|---|---|
| | 0 | 11 | 12 | 15 | 15 |
| Acetonitrile concentration % | 5 | 35 | 95 | 95 | 5 |

Method 6:

| | Time minutes | | | | |
|---|---|---|---|---|---|
| | 0 | 1.5 | 12 | 15 | 15.5 |
| Acetonitrile concentration % | 5 | 50 | 95 | 95 | 5 |

Method 7:

| | Time minutes | | | | |
|---|---|---|---|---|---|
| | 0 | 11 | 11.5 | 14.5 | 15 |
| Acetonitrile concentration % | 5 | 55 | 95 | 95 | 5 |

Proton and carbon NMR were acquired on a Bruker Advance 300 at 300 and 75 mHz respectively.

### Intermediate 1: tert-butyl 3-hydroxy-8-azabicyclo[3.2.1]octane-8-carboxylate

For a preparation, see WO2007/063071.

### Intermediate 2: benzyl 3-(hydroxymethyl)pyrrolidine-1-carboxylate

Pyrrolidin-3-ylmethanol (1.613 g, 15.95 mmol; Atlantic Research) and triethylamine (4.49 mL, 31.89 mmol) were stirred in dichloromethane at 0° C. Benzyl chloroformate (4.00 mL, 23.92 mmol) was added and the reaction allowed to warm to room temperature over 1 hour. The reaction mixture was diluted with DCM, washed with water, dried over sodium sulfate and concentrated at reduced pressure to afford the title compound (4.59 g). ¹H NMR (CDCl₃): δ=1.41-1.60 (1H, m), 1.62-1.80 (1H, m), 1.95-2.09 (1H, m), 2.35-2.52 (1H, m), 3.14-3.25 (1H, m), 3.37-3.70 (4H, m), 5.12 (2H, s), 7.28-7.40 (5H, m).

### Intermediate 3: tert-butyl 4-(2-pyrrolidin-1-ylethoxy)piperidine-1-carboxylate

To a stirred mixture of tert-butyl 4-(hydroxymethyl)piperidine-1-carboxylate (7.54 g, 35.0 mmol; Apollo), TBAB (1.13 g, 3.5 mmol) and 1-(2-chloroethyl)pyrrolidine hydrochloride (12.00 g, 70.0 mmol; Alfa Aesar) in toluene (60 mL) was added 10 M aqueous sodium hydroxide solution (60 mL). The resulting mixture was heated at 80° C. for 16 h. The reaction mixture was diluted with EtOAc (100 mL) and the organic phase separated. The aqueous phase was extracted with EtOAc (2×50 mL) and the combined organic phases were washed with brine (20 mL), separated, dried (magnesium sulfate), filtered and concentrated under reduced pressure to give an orange oil (14.77 g). The impure product was purified by flash column chromatography (silica gel, SNAP 100 g, gradient elution: DCM to 10% MeOH/DCM) to give the title compound as a yellow oil (8.31 g, 26.6 mmol, 76%). m/z [M+H]f 313.1. Retention time 3.52 min (LCMS method +ve 10 min).

The following intermediates 4 to 8 were prepared by a similar procedure to that used for intermediate 3 from the appropriate alcohol.

| Inter- | | |
|---|---|---|
| mediate | Name | Characterisation |
| 4 | tert-butyl 5-(2-pyrrolidin-1-ylethoxy)- | m/z [M + H]⁺ 311.2. |
| | 2-azabicyclo[2.2.1]heptane-2- | Retention time 0.81 min |
| | carboxylate | (LCMS method + |
| | | ve 6 min) |
| 5 | tert-butyl 3-(2-pyrrolidin-1-ylethoxy)- | ¹H NMR (CDCl₃) δ = 1.46 |
| | 8-azabicyclo[3.2.1]octane-8- | (9H, s), 1.74-2.11 (12H, |
| | carboxylate | m), 2.55-2.78 (6H, m), |
| | | 3.52-3.65 (3H, m), |
| | | 4.04-4.25 (2H, m). |
| 6 | tert-butyl 3-(2-pyrrolidin-1- | m/z [M + H]⁺ 313.2. |
| | ylethoxymethyl)piperidine-1- | Retention time 2.88 min |
| | carboxylate | (LCMS method + ve |
| | | 6 min). |
| 7 | benzyl 3-(2-pyrrolidin-1- | m/z [M + H]⁺ 373.2. |
| | ylethoxymethyl)-8- | Retention time 3.00 min |
| | azabicyclo[3.2.1]octane-8-carboxylate | (LCMS method + ve |
| | | 6 min). |
| 8 | benzyl 3-(2-pyrrolidin-1- | m/z [M + H]⁺ 333.2. |
| | ylethoxymethyl)pyrrolidine-1- | Retention time 2.98 min |
| | carboxylate | (LCMS method + ve |
| | | 6 min) |

### Intermediate 9: 4-(2-pyrrolidin-1-ylethoxymethyl)piperidine

To a stirred mixture of tert-butyl 4-(2-pyrrolidin-1-ylethoxymethyl)piperidine-1-carboxylate (8.31 g, 26.5 mmol; may be prepared as described in intermediate 3) in dichloromethane (60 mL) was cautiously added neat trifluoroacetic acid (30 mL) and the resulting mixture stirred at room temperature for 2 h. The reaction mixture was diluted with dichloromethane (30 mL) and water (30 mL) and the pH adjusted 12 using 10 M aqueous NaOH. The organic phase was separated and the aqueous phase extracted with DCM (3×50 mL). The combined organic phases were washed with brine (20 ml), separated, dried (magnesium sulfate), filtered and concentrated under reduced pressure to give the title compound as a viscous orange/dark yellow oil (4.79 g, 22.6 mmol, 85%). m/z [M+H]⁺ 213.1. Retention time 0.58 min (LCMS method +ve 10 min).

The following intermediates 10 to 12 were prepared by a similar procedure to that used for intermediate 9 from the appropriate tert-butoxycarbonyl-protected amine.

| Inter- | | |
|---|---|---|
| mediate | Name | Characterisation |
| 10 | 5-(2-pyrrolidin-1- | m/z [M + H]⁺ 211.2. Retention time |
| | ylethoxy)-2- | 0.55 min (LCMS method + ve 6 min). |
| | azabicyclo[2.2.1]heptane | |
| 11 | 3-(2-pyrrolidin-1- | m/z [M + H]⁺ 225.1. Retention time |
| | ylethoxy)-8- | 0.56 min (LCMS method + ve 6 min). |
| | azabicyclo[3.2.1]octane | |
| 12 | 3-(2-pyrrolidin-1- | m/z [M + H]⁺ 213.2. Retention time |
| | ylethoxymethyl)piperidine | 0.54 min (LCMS method + ve 6 min). |

### Intermediate 13: 3-(2-pyrrolidin-1-ylethoxymethyl)-8-azabicyclo[3.2.1]octane

Benzyl 3-(2-pyrrolidin-1-ylethoxymethyl)-8-azabicyclo[3.2.1]octane-8-carboxylate (1.2 g, 3.2 mmol; may be prepared as described in intermediate 7) and 10% palladium on carbon (0.12 g, 1.1276 mmol) were stirred in ethanol (20 mL) under an atmosphere of hydrogen overnight. The reaction mixture was filtered through Celite, washing the catalyst with ethanol. The filtrate was concentrated at reduced pressure to afford the title compound (0.792 g). m/z [M+H]f 239.2. Retention time 0.56 min (LCMS method +ve 6 min).

The following intermediate 14 was prepared by a similar procedure to that used for intermediate 13 from the appropriate benzyloxycarbonyl-protected amine.

| Intermediate | Name | Characterisation |
|---|---|---|
| 14 | 1-[2-(pyrrolidin-3-ylmethoxy) | m/z [M + H]⁺ 199.2. |
| | ethyl]pyrrolidine | Retention time 0.83 min |
| | | (LCMS method +ve 6 min). |

### Intermediate 15: ethyl 2,5-dibromo-4-phenyl-thiophene-3-carboxylate

To a stirred solution of copper (II) bromide (1.4 equiv., 84.92 mmol) in ACN (200 mL, 3830 mmol) at 0° C. was slowly added tert-butyl nitrite (1.15 equiv., 69.75 mmol). The reaction was stirred at 0° C. for 15 minutes before ethyl 2-amino-4-phenyl-thiophene-3-carboxylate (15 g, 60.66 mmol; Fluorochem) was added portionwise. The reaction mixture was allowed to warm to room temperature and stirred overnight. The reaction was partitioned between 2M HCl (200 mL) and ethyl acetate (200 mL) and further extracted with ethyl acetate (2×100 mL). The combined organics were dried over sodium sulfate and concentrated in vacuo. The residue was purified by dry flash column chromatography using silica and hexane: ethyl acetate 0-20% as eluent. The pure fractions were combined and concentrated to give the title compound (7.587 g, 40%). ¹H NMR (CDCl₃) 6=0.98 (3H, t), 4.08 (2H, q), 7.21-7.27 (2H, m), 7.36-7.45 (3H, m). Retention time 5.20 min (LCMS method +ve 6 min).

The following intermediate 16 was prepared by a similar procedure to that used for intermediate 15 from the appropriate thiophene compound.

| Intermediate | Name | Characterisation |
|---|---|---|
| 16 | ethyl 2,5-dibromo-4-(4-fluorophenyl) | ¹H NMR (CDCl₃) δ = 1.04 (3H, t), 4.12 |
| | thiophene-3-carboxylate | (2H, q), 7.08 − 7.14 (2H, m), 7.21 − 7.27 (2H, m). |
| | | Retention time 5.08 min |
| | | (LCMS method +ve 6 min). |

### Intermediate 17: 2,5-dibromo-4-phenyl-thiophene-3-carboxylic acid

Ethyl 2,5-dibromo-4-phenyl-thiophene-3-carboxylate (3.5 g, 11 mmol; may be prepared as described in intermediate 15) and potassium hydroxide (1.3 g, 22 mmol) were stirred in ethanol/water (10 mL/10 mL) at 50° C. for 3 hours. The reaction mixture was acidified to pH7, extracted into DCM, dried over sodium sulfate and concentrated at reduced pressure to afford the title compound (3.35 g). m/z [M+H]f 362.7. Retention time 4.60 min (LCMS method +ve 6 min).

The following intermediate 18 was prepared by a similar procedure to that used for intermediate 17 from the appropriate ester.

| Intermediate | Name | Characterisation |
|---|---|---|
| 18 | 2,5-dibromo-4-(4-fluorophenyl) | Retention time 4.57 min (LCMS |
| | thiophene-3-carboxylic acid | method +ve 6 min) |

### Intermediate 19: 2,5-dibromo-4-phenyl-thiophene-3-carbonyl chloride

2,5-Dibromo-4-phenyl-thiophene-3-carboxylic acid (3.5 g, 9.7 mmol; may be prepared as described in intermediate 17) was stirred in DCM (25 mL) with a drop of NMP. Thionyl chloride (1.3 g, 0.78 mL, 11 mmol) was added and the reaction heated to reflux for 2 hours. The solvent was removed at reduced pressure to afford the title compound (3.32 g). Retention time 5.30 min (LCMS method +ve 6 min).

The following intermediate 20 was prepared by a similar procedure to that used for intermediate 19 from the appropriate carboxylic acid.

| Intermediate | Name | Characterisation |
|---|---|---|
| 20 | 2,5-dibromo-4-(4- | ¹H NMR (CDCl₃) |
| | fluorophenyl)thiophene-3- | δ = 7.12 − 7.18 |
| | carbonyl chloride | (2H, m), 7.26 −7.30 (2H, m). |

### Intermediate 21: 2,5-dibromo-4-phenyl-N′-(p-tolylsulfonyl)thiophene-3-carbohydrazide

2,5-Dibromo-4-phenyl-thiophene-3-carbonyl chloride (3.32 g, 8.73 mmol; may be prepared as described in intermediate 19) and 4-methylbenzenesulfonohydrazide (3.25 g, 17.5 mmol) were heated to 100° C. in toluene (50 mL) for 2 hours. The reaction mixture was allowed to cool to room temperature and the suspension filtered. The solid was slurried with 1N HCl and the suspension filtered. The solid was washed with water and dried in vacuo at 40° C. overnight to afford the title compound (5.32 g). m/z [M+H]f 530.9. Retention time 4.39 min (LCMS method +ve 6 min).

The following intermediates 22 to 23 were prepared by a similar procedure to that used for intermediate 21 from the appropriate acid chlorides and hydrazides.

| Intermediate | Name | Characterisation |
|---|---|---|
| 22 | 2,5-dibromo-4-(4-fluorophenyl)- | m/z [M + H]⁺ 548.7. Retention time 4.40 |
| | N′-(p-tolylsulfonyl)thiophene-3- | min (LCMS method +ve 6 min). |
| | carbohydrazide | |
| 23 | N′-(benzenesulfonyl)-2,5- | m/z [M + H]⁺ 516.7. Retention time 4.38 |
| | dibromo-4-phenyl-thiophene-3- | min (LCMS method +ve 6 min). |
| | carbohydrazide | |

### Intermediate 24: (3Z)-2,5-dibromo-4-phenyl-N-(p-tolylsulfonyl)thiophene-3-carbohydrazonoyl chloride

2,5-Dibromo-4-phenyl-N′-(p-tolylsulfonyl)thiophene-3-carbohydrazide (5.32 g, 10.0 mmol; may be prepared as described in intermediate 21) was heated to 80° C. in thionyl chloride (7.18 g, 4.40 mL, 60.2 mmol) for 1 hour. The reaction mixture was allowed to cool to room temperature. Hexane (50 mL) was added and the resulting precipitate filtered off and dried in vacuo at 40° C. overnight (title compound; 3.8 g). m/z [M+H]⁺ 552.8. Retention time 4.41 min (LCMS method +ve 6 min).

The following intermediates 25 to 26 were prepared by a similar procedure to that used for intermediate 24 from the appropriate carbohydrazides.

| Intermediate Name | Characterisation | |
|---|---|---|
| 25 | (3Z)-2,5-dibromo-4-(4- | m/z [M + H]⁺ 570.7. Retention time |
| | fluorophenyl)-N-(p- | 4.42 min (LCMS method +ve 6 min). |
| | tolylsulfonyl)thiophene-3- | |
| | carbohydrazonoyl chloride | |
| 26 | (3Z)-N-(benzenesulfonyl)-2,5- | m/z [M + H]⁺ 538.7. Retention time |
| | dibromo-4-phenyl-thiophene-3- | 4.39 min (LCMS method +ve 6 min). |
| | carbohydrazonoyl chloride | |

### Intermediate 27: N—[(Z)-[(2,5-dibromo-4-phenyl-3-thienyl)-[4-(2-pyrrolidin-1-ylethoxymethyl)-1-piperidyl]methylene]amino]-4-methyl-benzenesulfonamide

(3Z)-2,5-dibromo-4-phenyl-N-(p-tolylsulfonyl)thiophene-3-carbohydrazonoyl chloride (2 g, 3.645 mmol; may be prepared as described in intermediate 24) was stirred in THF (30 mL) at room temperature. DABCO (0.8178 g, 0.802 mL, 7.290 mmol) and 4-(2-pyrrolidin-1-ylethoxymethyl)piperidine (1.161 g, 5.467 mmol; may be prepared as described in intermediate 9) were added and the reaction stirred overnight at room temperature. The reaction mixture was diluted with DCM (100 mL), washed with water (100 mL), dried over sodium sulfate and concentrated to afford the title compound (1.4 g). m/z [M/2+H]⁺ 363.0. Retention time 3.35 min (LCMS method +ve 6 min).

The following intermediates 28 to 35 were prepared by a similar procedure to that used for intermediate 27 from the appropriate carbohydrazonoyl chlorides and amines.

| Intermediate | Name | Characterisation |
|---|---|---|
| 28 | N-[(Z)-[(2,5-dibromo-4-phenyl-3-thienyl)- | m/z [M + H]⁺ 722.7. |
| | [5-(2-pyrrolidin-1-ylethoxy)-2- | Retention time 4.39 min |
| | azabicyclo[2.2.1]heptan-2- | (LCMS method +ve 6 min). |
| | yl]methylene]amino]-4-methyl- | |
| | benzenesulfonamide | |
| 29 | N-[(Z)-[[2,5-dibromo-4-(4-fluorophenyl)-3- | m/z [M + H]⁺ 741.0. |
| | thienyl]-[5-(2-pyrrolidin-1-ylethoxy)-2- | Retention time 3.30 min |
| | azabicyclo[2.2.1]heptan-2- | (LCMS method +ve 6 min). |
| | yl]methylene]amino]-4-methyl- | |
| | benzenesulfonamide | |
| 30 | N-[(Z)-[(2,5-dibromo-4-phenyl-3-thienyl)- | m/z [M + H]⁺ 750.9. |
| | [3-(2-pyrrolidin-1-ylethoxymethyl)-8- | Retention time 3.36 min |
| | azabicyclo[3.2.1]octan-8- | (LCMS method +ve 6 min) |
| | yl]methylene]amino]-4-methyl- | |
| | benzenesulfonamide | |
| 31 | N-[(Z)-[(2,5-dibromo-4-phenyl-3-thienyl)- | m/z [M + H]⁺ 737.0. |
| | [3-(2-pyrrolidin-1-ylethoxy)-8- | Retention time 0.81 min |
| | azabicyclo[3.2.1]octan-8- | (LCMS method +ve 6 min) |
| | yl]methylene]amino]-4-methyl- | |
| | benzenesulfonamide | |
| 32 | N-[(Z)-[(2,5-dibromo-4-phenyl-3-thienyl)- | m/z [M + H]⁺ 725.0. |
| | [3-(2-pyrrolidin-1-ylethoxymethyl)-1- | Retention time 3.53 min |
| | piperidyl]methylene]amino]-4-methyl- | (LCMS method +ve 6 min) |
| | benzenesulfonamide | |
| 33 | N-[(Z)-[(2,5-dibromo-4-phenyl-3-thienyl)- | m/z [M + H]⁺ 710.9. |
| | [3-(2-pyrrolidin-1- | Retention time 3.36 min |
| | ylethoxymethyl)pyrrolidin-1- | (LCMS method +ve 6 min) |
| | yl]methylene]amino]-4-methyl- | |
| | benzenesulfonamide | |
| 34 | N-[(Z)-[(2,5-dibromo-4-phenyl-3-thienyl)- | m/z [M + H]⁺ 710.9. |
| | [4-(2-pyrrolidin-1-ylethoxymethyl)-1- | Retention time 3.35 min |
| | piperidyl]methylene]amino] | (LCMS method +ve 6 min) |
| | benzenesulfonamide | |
| 35 | N-[(Z)-[(2,5-dibromo-4-phenyl-3-thienyl)- | m/z [M + H]⁺ 625.8. |
| | (3-hydroxy-8-azabicyclo[3.2.1]octan-8- | Retention time 6.50 min |
| | yl)methylene]amino]benzenesulfonamide | (LCMS method +ve 10 min) |

### Intermediate 36: 4-phenyl-1-(p-tolylsulfonyl)-3-[4-(2-pyrrolidin-1-ylethoxymethyl)-1-piperidyl]thieno[2,3-c]pyrazole

N—[(Z)-[(2,5-dibromo-4-phenyl-3-thienyl)-[4-(2-pyrrolidin-1-ylethoxymethyl)-1-piperidyl]methylene]amino]-4-methyl-benzenesulfonamide (1.37 g, 1.89 mmol; may be prepared as described in intermediate 27), copper(I) iodide (0.0360 g, 0.00641 mL, 0.189 mmol), potassium carbonate (0.523 g, 3.78 mmol) in NMP (2 mL) were heated to 180° C. in the microwave for 1 hour. The reaction mixture was diluted with ethyl acetate (50 mL) and filtered through celite. The filtrate was washed with water (50 mL), dried over Na2SO4 and concentrated at reduced pressure. The resulting residue was purified by flash chromatography, eluting with a gradient of DCM-93/7/0.7 DCM/MeOH/NH₄OH to afford the title compound (264 mg). m/z [M+H]⁺ 565.2. Retention time 3.42 min (LCMS method +ve 6 min).

### Intermediate 37: 5-bromo-4-phenyl-1-(p-tolylsulfonyl)-3-[5-(2-pyrrolidin-1-ylethoxy)-2-azabicyclo[2.2.1]heptan-2-yl]thieno[2,3-c]pyrazole

N—[(Z)-[(2,5-dibromo-4-phenyl-3-thienyl)-[5-(2-pyrrolidin-1-ylethoxy)-2-azabicyclo[2.2.1]heptan-2-yl]methylene]amino]-4-methyl-benzenesulfonamide (3 g, 4.152 mmol; may be prepared as described in intermediate 28), copper(I) iodide (0.0141 mL, 0.4152 mmol), potassium carbonate (1.148 g, 8.303 mmol) were heated to 100° C. in the microwave for 15 mins. The reaction mixture was diluted with ethyl acetate and filtered through Celite. The filtrate was washed with water, dried over sodium sulfate and concentrated at reduced pressure. The resulting residue was purified by flash chromatography, eluting with a gradient of DCM-93/3/0.3 DCM/MeOH/NH₄OH to afford the target compound (1.354 g). m/z [M+H]⁺ 641.1/643.1. Retention time 0.83 min (LCMS method +ve 6 min).

The following intermediates 38 to 44 were prepared by a similar procedure to that used for intermediate 37 from the appropriate sulfonamide-amidines.

| Intermediate | Name | Characterisation |
|---|---|---|
| 38 | 5-bromo-4-(4-fluorophenyl)-1-(p- | m/z [M + H]⁺ 659.0/660.9. |
| | tolylsulfonyl)-3-[5-(2-pyrrolidin-1- | Retention time 3.54 min |
| | ylethoxy)-2-azabicyclo[2.2.1]heptan-2- | (LCMS method +ve 6 min) |
| | yl]thieno[2,3-c]pyrazole | |
| 39 | 5-bromo-4-phenyl-1-(p-tolylsulfonyl)-3-[3- | m/z [M + H]⁺ 669.0/671.0. |
| | (2-pyrrolidin-1-ylethoxymethyl)-8- | Retention time 3.60 min |
| | azabicyclo[3.2.1]octan-8-yl-thieno[2,3- | (LCMS method +ve 6 min) |
| | c]pyrazole | |
| 40 | 5-bromo-4-phenyl-1-(p-tolylsulfonyl)-3-[3- | m/z [M + H]⁺ 655.1/657.1. |
| | (2-pyrrolidin-1-ylethoxy)-8- | Retention time 3.27 min |
| | azabicyclo[3.2.1]octan-8-yl-]thieno[2,3- | (LCMS method +ve 6 min) |
| | c]pyrazole | |
| 41 | 5-bromo-4-phenyl-1-(p-tolylsulfonyl)-3-[3- | m/z [M + H]⁺ 643.1/645.1. |
| | (2-pyrrolidin-1-ylethoxymethyl)-1- | Retention time 3.91 min |
| | piperidyl]thieno[2,3-c]pyrazole | (LCMS method +ve 6 min) |
| 42 | 5-bromo-4-phenyl-1-(p-tolylsulfonyl)-3-[3- | m/z [M + H]⁺ 629.0/631.0. |
| | (2-pyrrolidin-1-ylethoxymethyl) | Retention time 3.54 min |
| | pyrrolidin-1-yl]thieno[2,3-c]pyrazole | (LCMS method +ve 6 min) |
| 43 | 1-(benzenesulfonyl)-5-bromo-4-phenyl-3- | m/z [M + H]⁺ 629.0/631.0. |
| | [4-(2-pyrrolidin-1-ylethoxymethyl)-1- | Retention time 3.54 min |
| | piperidyl]thieno[2,3-c]pyrazole | (LCMS method +ve 10 min) |
| 44 | 8-[1-(benzenesulfonyl)-5-bromo-4-phenyl- | m/z [M + H]⁺ 543.9/545.9. |
| | thieno[2,3-c]pyrazol-3-yl]-8- | Retention time 5.06 min |
| | azabicyclo[3.2.1]octan-3-ol | (LCMS method +ve 6 min) |

### Intermediate 45: 8-[1-(benzenesulfonyl)-5-bromo-4-phenyl-thieno[2,3-c]pyrazol-3-yl]-8-azabicyclo[3.2.1]octan-3-one

To a stirred solution of 8-[1-(benzenesulfonyl)-5-bromo-4-phenyl-thieno[2,3-c]pyrazol-3-yl]-8-azabicyclo[3.2.1]octan-3-ol (980 mg, 1.800 mmol; may be prepared as described in intermediate 44) in dichloromethane (30 mL) at room temperature was added Dess-Martin Periodinane (1.3 equiv., 2.340 mmol) in one portion and the reaction stirred over the weekend. The reaction was filtered and the filtrate washed with water and brine, dried over sodium sulfate, filtered and concentrated in vacuo to yield the title compound (960 mg). m/z [M+H]⁺ 541.9/543.9. Retention time 5.14 min (LCMS method +ve 6 min).

### Intermediate 46: 1-(benzenesulfonyl)-4-phenyl-3-[4-(2-pyrrolidin-1-ylethoxymethyl)-1-piperidyl]thieno[2,3-c]pyrazole-5-carbonitrile

In a sealed microwave tube nitrogen was bubbled through a stirred solution of 1-(benzenesulfonyl)-5-bromo-4-phenyl-3-[4-(2-pyrrolidin-1-ylethoxymethyl)-1-piperidyl]thieno[2,3-c]pyrazole (6.00 g, 9.530 mmol; may be prepared as described in intermediate 43), zinc cyanide (1.287 g, 10.96 mmol), and diphenylphosphino ferrocene (0.545 g, 0.953 mmol) in DMF (30 mL) for 30 minutes at room temperature. To the stirred reaction was added tris(dibenzylideneacetone) dipalladium (0.436 g, 0.477 mmol), the vessel sealed and heated in a microwave reactor at 140° C. for 60 minutes. The reaction was diluted with ethyl acetate (200 mL) and water (200 mL), the organic layer washed with brine, dried over sodium sulphate, filtered and concentrated under reduced pressure. The material was passed through a pad of silica eluting with DCM: methanol (0-20%) as eluent and concentrated to give the named product (5.01 g, ⁹1%). m/z [M+H]⁺ 576.1. Retention time 3.42 min (LCMS method +ve 6 min).

### Intermediate 47: 5-bromo-4-phenyl-3-[4-(2-pyrrolidin-1-ylethoxymethyl)-1-piperidyl]-1H-thieno[2,3-c]pyrazole

The title compound was made in a similar manner to the preparation of compound 1, replacing 4-phenyl-1-(p-tolylsulfonyl)-3-[4-(2-pyrrolidin-1-ylethoxymethyl)-1-piperidyl]thieno[2,3-c]pyrazole with 1-(benzenesulfonyl)-5-bromo-4-phenyl-3-[4-(2-pyrrolidin-1-ylethoxymethyl)-1-piperidyl]thieno[2,3-c]pyrazole (may be prepared as described in intermediate 43). m/z [M+H]⁺ 489.0/491.0. Retention time 3.30 min (LCMS method +ve 6 min).

### Intermediate 48: 5-bromo-1-methylsulfonyl-4-phenyl-3-[4-(2-pyrrolidin-1-ylethoxymethyl)-1-piperidyl]thieno[2,3-c]pyrazole

The title compound was made in a similar manner to the preparation of compound 13, replacing 4-phenyl-3-[4-(2-pyrrolidin-1-ylethoxymethyl)-1-piperidyl]-1H-thieno[2,3-c]pyrazole with 5-bromo-4-phenyl-3-[4-(2-pyrrolidin-1-ylethoxymethyl)-1-piperidyl]-1H-thieno[2,3-c]pyrazole (may be prepared as described in intermediate 47), and acetyl chloride with methanesulfonyl chloride. m/z [M+H]⁺ 566.9/569.0. Retention time 3.38 min (LCMS method +ve 6 min).

### Intermediate 49: 1-methyl-4-phenyl-3-[4-(2-pyrrolidin-1-ylethoxymethyl)-1-piperidyl]thieno[2,3-c]pyrazole-5-carbonitrile

To a stirred solution of 4-phenyl-3-[4-(2-pyrrolidin-1-ylethoxymethyl)-1-piperidyl]-1H-thieno[2,3-c]pyrazole-5-carbonitrile (140 mg, 0.3214 mmol; may be prepared as described in compound 3) in THF (10 mL, 123 mmol) at room temperature was added potassium tert-butoxide (1.15 equiv., 0.3696 mmol) in one portion and the reaction stirred for 15 mins. The reaction was cooled to −30° C. and methyl iodide was added, 1 equivalent initially, followed by a second equivalent after 1 hr. The reaction was stirred for 2 hr at room temperature. The reaction was diluted with water and ethyl acetate, washed with brine, dried over sodium sulfate, filtered and concentrated to give the title compound (110 mg). m/z [M+H]f 450.1. Retention time 3.27 min (LCMS method +ve 6 min).

### Intermediate 50: 1-methyl-4-phenyl-3-[4-(2-pyrrolidin-1-ylethoxymethyl)-1-piperidyl]thieno[2,3-c]pyrazole-5-carboxylic acid

1-Methyl-4-phenyl-3-[4-(2-pyrrolidin-1-ylethoxymethyl)-1-piperidyl]thieno[2,3-c]pyrazole-5-carbonitrile (110 mg, 0.2447 mmol; may be prepared as described in intermediate 49), 2M sodium hydroxide (2 mL) and methanol (3 mL) were placed in a microwave vial and heated at 125° C. for 2 hours, and then for 20 mins at 110° C. The methanol was removed under reduced pressure and the reaction neutralised with sulfuric acid. The reaction was diluted with water and DCM, extracted with DCM and the layers separated. The aqueous layer was passed through a 103 catch and release cartridge eluting with MeOH to give the title compound (18 mg). m/z [M+H]⁺ 469.1. Retention time 3.93 min (LCMS method +ve 6 min).

### Intermediate 51: 4-phenyl-3-[4-(2-pyrrolidin-1-ylethoxymethyl)-1-piperidyl]-1H-thieno[2,3-c]pyrazole-5-carboxylic acid

A mixture of 1-(benzenesulfonyl)-4-phenyl-3-[4-(2-pyrrolidin-1-ylethoxymethyl)-1-piperidyl]thieno[2,3-c]pyrazole-5-carbonitrile (0.500 g, 0.8685 mmol; may be prepared as described in intermediate 46), lithium hydroxide (0.365 g, 8.685 mmol), methanol (5 mL) and water (5 mL) were placed in a microwave vial and heated at 130° C. for 3 hours. The methanol was removed under reduced pressure and the reaction acidified to pH 4 with sulfuric acid. The reaction was further diluted with water and DCM, extracted with CHCl₃:IPA 3:1 and the layers separated. The organic layer was dried over sodium sulfate, filtered and concentrated in vacuo. The crude product was purified by prep chromatography using acidic eluent and concentrated to afford the title compound (0.170 g, 43%). m/z [M+H]⁺ 454.1. Retention time 2.86 min (LCMS method +ve 6 min).

### Intermediate 52: methyl 4-phenyl-3-[4-(2-pyrrolidin-1-ylethoxymethyl)-1-piperidyl]-1H-thieno[2,3-c]pyrazole-5-carboxylate

A mixture of 4-phenyl-3-[4-(2-pyrrolidin-1-ylethoxymethyl)-1-piperidyl]-1H-thieno[2,3-c]pyrazole-5-carboxylic acid (0.120 g, 0.264 mmol; may be prepared as described in intermediate 51), methanol (3 mL) and sulphuric acid (1 mL) were placed in a microwave vial and heated at 100° C. for 2 hours. The methanol was removed under reduced pressure and the reaction poured into saturated bicarbonate solution (10 mL) and DCM (10 mL). The reaction was further extracted with DCM (2×10 mL), dried over sodium sulphate, filtered and concentrated under reduced pressure to afford the named product (0.098 g, 80%). m/z [M+H]⁺ 469.1. Retention time 3.09 min (LCMS method +ve 6 min).

### Compound 1: 4-phenyl-3-[4-(2-pyrrolidin-1-ylethoxymethyl)-1-piperidyl]-1H-thieno[2,3-c]pyrazole

4-Phenyl-1-(p-tolylsulfonyl)-3-[4-(2-pyrrolidin-1-ylethoxymethyl)-1-piperidyl]thieno[2,3-c]pyrazole (0.26 g, 0.4603 mmol; may be prepared as described in intermediate 36) and potassium hydroxide (0.1291 g, 2.302 mmol) were combined in methanol (5 mL) and heated to reflux for 30 minutes. The solvent was removed at reduced pressure. The resulting residue was taken up in DCM (50 mL) washed with water (50 mL), dried over sodium sulfate and concentrated at reduced pressure. The residue was purified by basic prep HPLC (method 6) to afford the title compound (45 mg). ¹H NMR (CDCl₃): δ=1.28 (2H, qd), 1.6-1.8 (8H, m), 2.48-2.72 (8H, m), 3.32 (2H, d), 3.04 (3H, d), 3.36 (1H, br), 3.56 (2H, t), 6.80 (1H, s), 7.32 (1H, d) 7.44 (2H, t), 7.72 (2H, d). m/z [M+H]⁺ 411.1. Retention time 3.05 min (LCMS method +ve 6 min).

The following compounds 2 to 3 were prepared by a similar procedure to that used for compound 1 from the appropriate sulfonyl-protected thienopyrazoles.

| Compound | Name | Characterisation |
|---|---|---|
| 2 | 5-bromo-4-phenyl-3-[5-(2-pyrrolidin-1- | m/z [M +H]⁺ 487.1/489.1. |
| | ylethoxy)-2-azabicyclo[2.2.1]heptan-2- | Retention time 3.15 min |
| | yl]-1H-thieno[2,3-c]pyrazole | (LCMS method +ve 6 min) |
| 3 | 4-phenyl-3-[4-(2-pyrrolidin-1- | m/z [M + H]⁺ 436.1. |
| | ylethoxymethyl)-1-piperidyl]-1H- | Retentiontime 3.23 min |
| | thieno[2,3-c]pyrazole-5-carbonitrile | (LCMS method +ve 6 min) |

### Compound 4: 4-phenyl-3-[5-(2-pyrrolidin-1-ylethoxy)-2-azabicyclo[2.2.1]heptan-2-yl]-1H-thieno[2,3-c]pyrazole

5-Bromo-4-phenyl-1-(p-tolylsulfonyl)-3-[5-(2-pyrrolidin-1-ylethoxy)-2-azabicyclo[2.2.1]heptan-2-yl]thieno[2,3-c]pyrazole (0.800 g, 1.25 mmol, may be prepared as described in intermediate 37), triphenylphosphine (0.0661 g, 0.249 mmol), potassium carbonate (0.345 g, 2.49 mmol) and palladium(II) acetate (0.0140 g, 0.0623 mmol) were combined and heated in 1-butanol (5 mL) in the microwave at 150° C. for 30 minutes. The solvent was removed at reduced pressure. The resulting residue was taken up in DCM, washed with water, dried over sodium sulfate and concentrated at reduced pressure. The residue was purified by flash chromatography, eluting with DCM-90/10/1 DCM/MeOH/NH₄OH to afford the title compound (0.3 g). m/z [M+H]⁺ 409.2. Retention time 5.16 min (LCMS method +ve 6 min vv polar).

The following compound 5 was prepared by a similar procedure to that used for compound 4 from the appropriate sulfonyl-protected bromothienopyrazole.

| Compound | Name | Characterisation |
|---|---|---|
| 5 | 4-(4-fluorophenyl)-3-[5-(2-pyrrolidin-1- | m/z [M + H]⁺ 427.2. |
| | ylethoxy)-2-azabicyclo[2.2.1]heptan- | Retention time 5.14 min |
| | 2-yl]-1H-thieno | (LCMS method +ve 6 min vvpolar). |

### Compound 6: 4-phenyl-3-[3-(2-pyrrolidin-1-ylethoxymethyl)-8-azabicyclo[3.2.1]octan-8-yl]-1H-thieno[2,3-c]pyrazole

To a solution of 5-bromo-4-phenyl-1-(p-tolylsulfonyl)-3-[3-(2-pyrrolidin-1-ylethoxymethyl)-8-azabicyclo[3.2.1]octan-8-yl]thieno[2,3-c]pyrazole (250 mg, 0.3733 mmol; may be prepared as described in intermediate 39) and dibutyltin dichloride (0.2 equiv.) in THF (3 mL) in a capped microwave vial was added phenylsilane (1.25 equiv., 0.4666 mmol) in one portion. The reaction was heated in a microwave at 100° C. for 30 minutes. 3M sodium hydroxide solution (1.5 mL) was carefully added to the reaction and placed back in the microwave for 30 minutes at 100° C. The reaction was diluted with water and ethyl acetate, extracted with ethyl acetate, dried over sodium sulfate, filtered and concentrated in vacuo. The crude reaction was purified by LCUV using basic eluent, method 1. Product-containing fractions were combined and concentrated to give the title compound m/z [M+H]⁺ 437.1. Retention time 3.12 min (LCMS method +ve 6 min).

The following compounds 7 to 9 were prepared by a similar procedure to that used for compound 6 from the appropriate sulfonyl-protected bromothienopyrazoles.

| Compound | Name | Characterisation |
|---|---|---|
| 7 | 4-phenyl-3-[3-(2-pyrrolidin-1- | m/z [M + H]⁺ 423.2. |
| | ylethoxy)-8-azabicyclo[3.2.1]octan-8- | Retention time 5.17 min |
| | yl]-1H-thieno[2,3-c]pyrazole | (LCMS method +ve 6 min vvpolar) |
| 8 | 4-phenyl-3-[3-(2-pyrrolidin-1- | m/z [M + H]⁺ 411.1. |
| | ylethoxymethyl)-1-piperidyl[-1H- | Retention time 3.22 min |
| | thieno[2,3-c]pyrazole | (LCMS method +ve 6 min) |
| 9 | 4-phenyl-3-[3-(2-pyrrolidin-1- | m/z [M + H]⁺ 397.1. |
| | ylethoxymethyl)pyrrolidin-1-yl]-1H- | Retention time 3.41 min |
| | thieno[2,3-c]pyprazole | (LCMS method +ve 10 min). |

### Compound 10: N-cyclobutyl-8-(4-phenyl-1H-thieno[2,3-c]pyrazol-3-yl)-8-azabicyclo[3.2.1]octan-3-amine

A mixture of 8-[1-(benzenesulfonyl)-5-bromo-4-phenyl-thieno[2,3-c]pyrazol-3-yl]-8-azabicyclo[3.2.1]octan-3-one (200 mg, 0.3687 mmol; may be prepared as described in intermediate 45), cyclobutylamine (2 equiv., 0.7373 mmol), dibutyltin dichloride (0.2 equiv.) and phenylsilane (1.25 equiv., 0.4608 mmol) in THF (2 mL, 24.6 mmol) were combined in a microwave vial and the reaction heated at 100° C. for 30 minutes. The microwave vial was opened and 3M NaOH solution (2 mL) carefully added dropwise (some material lost as the reaction effervesced). The re-capped reaction was further heated in the microwave at 120° C. for 1 hour. The reaction was diluted with water (5 mL) and ethyl acetate (20 mL) and further extracted with ethyl acetate (2×10 mL). The combined extracts were washed with brine, dried over sodium sulfate, filtered and concentrated under reduced pressure. The crude material was purified by preparatory HPLC chromatography using basic eluent to give the title compound (39.5 mg). m/z [M+H]⁺ 379.1. Retention time 3.07 min (LCMS method +ve 6 min).

The following compounds 11 to 12 were prepared by a similar procedure to that used for compound 10 from the appropriate amines.

| Compound | Name | Characterisation |
|---|---|---|
| 11 | 3-[3-(azetidin-1-yl)-8- | m/z [M + H]⁺ 365.1. Retention time |
| | azabicyclo[3.2.1]octan-8-yl]-4- | 3.00 min (LCMS method +ve 6 min) |
| | phenyl-1H-thieno[2,3-c]pyrazole | |
| 12 | N-isopropyl-8-(4-phenyl-1H- | m/z [M + H]⁺ 367.1. Retention time |
| | thieno[2,3-c]pyrazol-3-yl)-8- | 3.06 min (LCMS method +ve 6 min) |
| | azabicyclo[3.2.1]octan-3-amine | |

### Compound 13: 1-[4-phenyl-3-[4-(2-pyrrolidin-1-ylethoxymethyl)-1-piperidyl]thieno[2,3-c]pyrazol-1-yl]ethanone

4-Phenyl-3-[4-(2-pyrrolidin-1-ylethoxymethyl)-1-piperidyl]-1H-thieno[2,3-c]pyrazole (35 mg, 0.085 mmol; may be prepared as described in compound 1) and triethylamine (0.024 mL, 0.17 mmol) were stirred at 0° C. in dichloromethane (10 mL). Acetyl chloride (0.009 mL, 0.13 mmol) was added and the reaction was allowed to warm to room temperature over 1 hour. The reaction mixture was washed with water, passed through a hydrophobic frit and concentrated at reduced pressure. The resulting residue was purified by basic prep HPLC (method 6) to afford the title compound (15 mg). m/z [M+H]⁺ 453.2. Retention time 5.27 min (LCMS method +ve 6 min).

The following compounds 14 to 19 were prepared by a similar procedure to that used for compound 13 from the appropriate thienopyrazoles and electrophiles.

| Compound | Name | Characterisation |
|---|---|---|
| 14 | 1-[4-phenyl-3-[5-(2-pyrrolidin-1- | m/z [M + H]⁺ 451.2. Retention |
| | ylethoxy)-2-azabicyclo[2.2.1]heptan-2- | time 5.23 min (LCMS method |
| | yl]thieno[2,3-c]pyrazol-1-yl]ethanone | +ve 6 min vv polar) |
| 15 | 1-[4-(4-fluorophenyl)-3-[5-(2-pyrrolidin-1- | m/z [M + H]⁺ 469.1. Retention |
| | ylethoxy)-2-azabicyclo[2.2.1]heptan-2- | time 5.22 min (LCMS method |
| | yl]thieno[2,3-c]pyrazol-1-yl]ethanone | +ve 6 min vv polar) |
| 16 | 4-(4-fluorophenyl)-1-methylsulfonyl-3-[5- | m/z +[M + H]⁺ 505.2. Retention |
| | (2-pyrrolidin-1-ylethoxy)-2- | time 3.21 min (LCMS method |
| | azabicyclo[2.2.1]heptan-2-yl]thieno[2,3- | +ve 6 min) |
| | c]pyrazole | |
| 17 | 1-[4-phenyl-3-[3-(2-pyrrolidin-1- | m/z [M + H]⁺ 465.2. Retention |
| | ylethoxy)-8-azabicyclo[3.2.1]octan-8- | time 5.20 min (LCMS method |
| | yl]thieno[2,3-c]pyrazol-1-yl]ethanone | +ve 6 min vv polar) |
| 18 | 1-[4-phenyl-3-[3-(2-pyrrolidin-1- | m/z [M + H]⁺ 453.1. Retention |
| | ylethoxymethyl)-1-piperidyl]thieno[2,3- | time 4.58 min (LCMS method |
| | c]pyrazol-1-yl]ethanone | +ve 10 min) |
| 19 | 1-[4-phenyl-3-[3-(2-pyrrolidin-1- | m/z [M + H]⁺ 439.1. Retention |
| | ylethoxymethyl)pyrrolidin-1-yl]thieno[2,3- | time 3.85 min (LCMS method |
| | c]pyrazol-1-yl]ethanone | +ve 10 min) |

### Compound 20: 1-methylsulfonyl-4-phenyl-3-[4-(2-pyrrolidin-1-ylethoxymethyl)-1-piperidyl]thieno[2,3-c]pyrazole

To a stirred solution of 5-bromo-1-methylsulfonyl-4-phenyl-3-[4-(2-pyrrolidin-1-ylethoxymethyl)-1-piperidyl]thieno[2,3-c]pyrazole (60 mg, 0.1057 mmol; may be prepared as described in intermediate 48), potassium carbonate (2 equiv., 0.2114 mmol), and triphenylphosphine (0.2 equiv., 0.02114 mmol) in n-butanol (2 mL) and ACN (2 mL) under nitrogen in a microwave vial was added palladium(II) acetate, trimer (0.05 equiv., 0.005285 mmol) in one portion and the tube sealed. The reaction was heated in a microwave for 30 min at 120° C. The reaction was concentrated then purified by LCUV (acidic method 1). The pure fractions were combined and concentrated, then passed through an SCX cartridge and eluted with MeOH/NH₃ to give the title compound (10 mg). m/z [M+H]⁺ 498.0. Retention time 3.24 min (LCMS method +ve 6 min).

### Compound 21: 1-methyl-4-phenyl-3-[4-(2-pyrrolidin-1-ylethoxymethyl)-1-piperidyl]thieno[2,3-c]pyrazole

1-Methyl-4-phenyl-3-[4-(2-pyrrolidin-1-ylethoxymethyl)-1-piperidyl]thieno[2,3-c]pyrazole-5-carbonitrile (110 mg, 0.2447 mmol; may be prepared as described in intermediate 49), 2M sodium hydroxide (2 mL) and methanol (3 mL) were placed in a microwave vial and heated at 125° C. for 2 hours, and then for 20 mins at 110° C. The methanol was removed under reduced pressure and the reaction neutralised with sulfuric acid. The reaction was diluted with water and DCM, extracted with DCM and the layers separated. The organic layer was concentrated in vacuo and the by-product purified by LCUV (acidic method 1). The dried pure sample was passed through an SCX cartridge and eluted with NH₃/MeOH to give the target compound (6 mg). m/z [M+H]⁺ 425.1. Retention time 3.22 min (LCMS method +ve 6 min).

### Compound 22: N,N,1-trimethyl-4-phenyl-3-[4-(2-pyrrolidin-1-ylethoxymethyl)-1-piperidyl]thieno[2,3-c]pyrazole-5-carboxamide

To a stirred solution of 1-methyl-4-phenyl-3-[4-(2-pyrrolidin-1-ylethoxymethyl)-1-piperidyl]thieno[2,3-c]pyrazole-5-carboxylic acid (0.018 g, 0.038 mmol; may be prepared as described in intermediate 50) in a mixture of DCM (1 mL) and DMF (2 mL) at room temperature was added HATU (0.029 g, 0.077 mmol) and 2M Dimethylamine (0.08 mL, 0.154 mmol) in THF. The reaction was stirred at room temperature over the course of a weekend, diluted with DCM (10 mL) and water (10 mL), the aqueous extracted with DCM (3×10 mL) and the combined organics dried over sodium sulfate, filtered and concentrated under reduced pressure. The crude reaction mixture was purified by LCUV (basic method 1) to give the title compound (0.0013 g, 0.029 mmol). m/z [M+H]f 496.1. Retention time 3.05 min (LCMS method +ve 6 min).

### Compound 23: N,N-dimethyl-4-phenyl-3-[4-(2-pyrrolidin-1-ylethoxymethyl)-1-piperidyl]-1H-thieno[2,3-c]pyrazole-5-carboxamide

To a stirred solution of methyl 4-phenyl-3-[4-(2-pyrrolidin-1-ylethoxymethyl)-1-piperidyl]-1H-thieno[2,3-c]pyrazole-5-carboxylate (0.098 g, 0.209 mmol; may be prepared as described in intermediate 52) and DCE (3 mL) in a microwave vial was added trimethyl aluminium 2M solution in hexane (0.314 mL, 0.628 mmol) and the reaction stirred at room temperature for 15 minutes. Dimethylamine 2M solution in THF (0.314 mL, 0.628 mmol) was added and the reaction heated in a microwave at 110° C. for 2 hours. The reaction was diluted with DCM (20 mL) and water (20 mL) and the organic phase separated. The aqueous phase was further extracted with DCM (2×10 mL) and the combined extracts dried over sodium sulphate, filtered and concentrated under reduced pressure. The crude material was purified by preparatory HPLC using acidic eluent and the pure fractions combined and evaporated under reduced pressure to afford the title compound as the formate salt (0.020 g, 20%). m/z [M+H]⁺ 482.2. Retention time 3.00 min (LCMS method +ve 6 min).

The following compounds 24 to 25 were prepared by a similar procedure to that used for compound 23 from the appropriate thienopyrazoles and amines.

| Compound | Name | Characterisation |
|---|---|---|
| 24 | N-isopropyl-4-phenyl-3-[4-(2-pyrrolidin-1- | m/z [M + H]⁺ 496.2. Retention |
| | ylethoxymethyl)-1-piperidyl]-1H-thieno[2,3- | time 3.04 min (LCMS |
| | c]pyrazole-5-carboxamide | method +ve 6 min) |
| 25 | N-methyl-4-phenyl-3-[4-(2-pyrrolidin-1- | m/z [M + H]⁺ 468.1. Retention |
| | ylethoxymethyl)-1-piperidyl]-1H-thieno[2,3- | time 2.96 min (LCMS |
| | c]pyrazole-5-carboxamide | method +ve 6 min) |

### Biological Testing

Compound activity against the recombinant G-protein activated inward rectifier current encoded by the heterotetramer Kir3.1/3.4 was assessed using manual whole-cell patch technique. The heterotetramer forms the pore-forming channel that conducts the acetylcholine/adenosine-activated potassium current in the heart.

### Kir3.1/3.4 Electrophysiology Method

For whole-cell patch-clamp studies, cells (Human Embryonic Kidney 293 stably transfected with rat Kir3.1/3.4) were seeded onto glass coverslips before recordings were made. Cells were seeded in sterile 30 mm Petri dishes at a density to enable isolated cells to be selected for patch clamp experiments. The dishes were stored in a humidified, gassed (5% CO₂) incubator at 37° C. until use.

Whole-cell patch-clamp recordings of membrane currents were made following gigaohm seal formation between the patch electrode and the cell using HEKA EPC-9/10 amplifiers controlled by Pulse Software (Ver8.5x/8.6x/8.7x, HEKA, Germany). Coverslips seeded with cells were placed in a recording chamber mounted on the stage of an inverted microscope. During the experiment, the cell of interest was continuously superfused with bather solution delivered via a cannula placed in close proximity to the cell to enable control of the extracellular solution environment. Only those cells with a current <−500 pA (current at −140 mV) were used for experiments. During experiments, series resistance was compensated by a minimum of 70%.

Electrophysiology voltage-step protocols and analysis of data were performed as follows. Data was sampled at 5 kHz, and filtered with a −3 dB bandwidth of 2.5 kHz. Cells were held at a voltage of −60 mV. Currents were evoked by a depolarising voltage step to +60 mV (100 ms) before a ramp-repolarisation (0.4 V·s⁻¹) to −140 mV (100 ms) before returning to −60 mV. The command waveform was repeatedly applied every 10 s throughout the experiment. Mean currents during 1-99% of the time at −140 mV were analysed using Pulsefit software (v8.x, HEKA, Germany). The voltage protocol was repeatedly applied to achieve a stable current baseline in bather before the test substance was superfused via the cannula in close proximity to the cell under investigation. The test substance was allowed to equilibrate during which time voltage protocol was repeatedly applied and recorded. On reaching steady-state inhibition, the cell was superfused with an identical bather solution containing zero external potassium chloride (replaced by equimolar NaCl). The identical current measurement was made in the absence of potassium to assess the passive leak at −140 mV. The leak current was subtracted from the control and steady-state drug current values. The percentage inhibition of the leak-subtracted current in the presence of test substance was calculated relative to the control leak-subtracted pre-drug value. Internal patch-pipette solution contained in mM: 110 KCl, 20 NaCl, 0.9 GTPγS, 5 Mg-ATP, 5 EGTA, 10 HEPES, pH7.2 corrected with KOH. The external superfusate composition in mM was: 150 (or 160) NaCl, 10 (or 0) KCl, 3 CaCl₂, 1 MgCl, 10 HEPES, pH 7.4 corrected with NaOH.

IC₅₀ data are provided in TABLE 1:

A corresponds to an IC₅₀ of less than 500 nM;
B corresponds to an IC₅₀ of greater than 500 nM but less than 3000 nM; and
C corresponds to an IC₅₀ of greater than 3000 nM but less than 10,000 nM.
A compound is considered to be “active” if its IC₅₀ is below 10,000 nM.

TABLE 1
Summary of biological activity

| Example | Chemical name | IC₅₀ |
|---|---|---|
| Compound | 4-phenyl-3-[4-(2-pyrrolidin-1-ylethoxymethyl)-1-piperidyl]- | A |
| 1 | 1H-thieno[2,3-c]pyrazole | |
| Compound | 5-bromo-4-phenyl-3-[5-(2-pyrrolidin-1-ylethoxy)-2- | A |
| 2 | azabicyclo[2.2.1]heptan-2-yl]-1H-thieno[2,3-c]pyrazole | |
| Compound | 4-phenyl-3-[4-(2-pyrrolidin-1-ylethoxymethyl)-1-piperidyl]- | A |
| 3 | 1H-thieno[2,3-c]pyrazole-5-carbonitrile | |
| Compound | 4-phenyl-3-[5-(2-pyrrolidin-1-ylethoxy)-2-azabicyclo[2.2.1] | A |
| 4 | heptan-2-yl]-1H-thieno[2,3-c]pyrazole | |
| Compound | 4-(4-fluorophenyl)-3-[5-(2-pyrrolidin-1-ylethoxy)-2- | A |
| 5 | azabicyclo[2.2.1]heptan-2-yl]-1H-thieno[2,3-c]pyrazole | |
| Compound | 4-phenyl-3-[3-(2-pyrrolidin-1-ylethoxymethyl)-8- | A |
| 6 | azabicyclo[3.2.1]octan-8-yl]-1H-thieno[2,3-c]pyrazole | |
| Compound | 4-phenyl-3-[3-(2-pyrrolidin-1-ylethoxy)-8-azabicyclo[3.2.1] | A |
| 7 | octan-8-yl]-1H-thieno[2,3-c]pyrazole | |
| Compound | 4-phenyl-3[3-(2-pyrrolidin-1-ylethoxymethyl)-1-piperidyl]- | A |
| 8 | 1H-thieno[2,3-c]pyrazole | |
| Compound | 4-phenyl-3-[3-(2-pyrrolidin-1-ylethoxymethyl)pyrrolidin-1- | A |
| 9 | yl]-1H-thieno[2,3-c]pyrazole | |
| Compound | N-cyclobutyl-8-(4-phenyl-1H-thieno[2,3-c]pyrazole-3-yl)- | A |
| 10 | 8-azabicyclo[3.2.1]octan-3-amine | |
| Compound | 3-[3-(azetidin-1-yl)-8-azabicyclo[3.2.1]octan-8-yl]-4- | B |
| 11 | phenyl-1H-thieno[2,3-c]pyrazole | |
| Compound | N-isopropyl-8-(4-phenyl-1H-thieno[2,3-c]pyrazole-3-yl)- | B |
| 12 | 8-azabicyclo[3.2.1]octan-3-amine | |
| Compound | 1-[4-phenyl-3-[4-(2-pyrrolidin-1-ylethoxymethyl)-1- | A |
| 13 | piperidyl]thieno[2,3-c]pyrazole-1-yl]ethanone | |
| Compound | 1-[4-phenyl-3-[5-(2-pyrrolidin-1-ylethoxy)-2-azabicyclo[2.2.1] | A |
| 14 | heptan-2-yl]thieno[2,3-c]pyrazole-1-yl]ethanone | |
| Compound | 1-[4-(4-fluorophenyl)-3-[5-(2-pyrrolidin-1-ylethoxy)-2- | A |
| 15 | azabicyclo[2.2.1]heptan-2-yl]thieno[2,3-c]pyrazol-1- | |
| | yl]ethanone | |
| Compound | 4-(4-fluorophenyl)-1-methylsulfonyl-3-[5-(2-pyrrolidin-1- | A |
| 16 | ylethoxy)-2-azabicyclo[2.2.1]heptan-2-yl]thieno[2,3- | |
| | c]pyrazole | |
| Compound | 1-[4-phenyl-3-[3-(2-pyrrolidin-1-ylethoxy)-8-azabicyclo[3.2.1] | A |
| 17 | octan-8-yl]thieno[2,3-c]pyrazol-1-yl]ethanone | |
| Compound | 1-[4-phenyl-3-[3-(2-pyrrolidin-1-ylethoxymethyl)-1- | A |
| 18 | piperidyl]thieno[2,3-c]pyrazol-1-yl]ethanone | |
| Compound | 1-[4-phenyl-3-[3-(2-pyrrolidin-1-ylethoxymethyl)pyrrolidin-1- | A |
| 19 | yl]thieno[2,3-c]pyrazol-1-yl]ethanone | |
| Compound | 1-methylsulfonyl-4-phenyl-3-[4-(2-pyrrolidin-1- | A |
| 20 | ylethoxymethyl)-1-piperidyl]thieno[2,3-c]pyrazole | |
| Compound | 1-methyl-4-phenyl-3-[4-(2-pyrrolidin-1-ylethoxymethyl)-1- | A |
| 21 | piperidyl]thieno[2,3-c]pyrazole | |
| Compound | N,N,1-trimethyl-4-phenyl-3-[4-(2-pyrrolidin-1-ylethoxymethyl)- | B |
| 22 | 1-piperidyl]thieno[2,3-c]pyrazole-5-carboxamide | |
| Compound | N,N-dimethyl-4-phenyl-3-[4-(2-pyrrolidin-1-ylethoxymethyl)- | B |
| 23 | 1-piperidyl]-1H-thieno[2,3-c]pyrazole-5-carboxamide | |
| Compound | N-isopropyl-4-phenyl-3-[4-(2-pyrrolidin-1-ylethoxymethyl)- | A |
| 24 | 1-piperidyl]-1H-thieno[2,3-c]pyrazole-5-carboxamide | |
| Compound | N-methyl-4-phenyl-3-[4-(2-pyrrolidin-1-ylethoxymethyl)- | A |
| 25 | 1-piperidyl]-1H-thieno[2,3-c]pyrazole-5-carboxamide | |

### REFERENCES

Berg, Tom Christian; Bakken, Vebjoern; Gundersen, Lise-Lotte; Petersen, Dirk Cyclization and rearrangement products from coupling reactions between terminal o-alkynylphenols or o-ethynyl(hydroxymethyl)benzene and 6-halopurines Tetrahedron, 2006, vol. 62, #25 p. 6121-6131.
Jingjun yin, Buchwald, Stephen L.; Palladium-Catalyzed Intermolecular Coupling of Aryl Halides and Amides; Org. Lett. 2000, Vol. 2, (8), p. 1101-1104.
Munchof et al., Design and SAR of thienopyrimidine and thienopyridine inhibitors of VEGFR-2 kinase activity. Bioorganic & Medicinal Chemistry Letters, 14(1), 21-24, 2004.
Barker et al., Thienopyridines Part 6. Synthesis and nucleophilic substitution of some chlorothieno[2,3b]pyridine derivatives and comparison with the analogous quinoline compounds. J. Chem Res. (Miniprint), 1985, 2501-2509.
Charvát et al., Diethyl Acetonedicarboxylate—a Precursor for the Synthesis of new Substituted 4-Aminoquinolines and Fused 4-Aminopyridines. Monatsheft. Chem. 126, 333-340, 1995.
Gewald et al., Synthesen von 4-Amino-thieno[2,3-b]pyridinen, Monatsheft. Chem. 110, 1189-1196, 1979.
Chinchilla, Rafael, and Njera, Carmen, The Sonogashira Reaction: A Booming Methodology in Synthetic Organic Chemistry; Chem. Rev., 2007, 107 (3), pp 874-922.
Greene et al., Protective groups in organic synthesis, 3^{rd} edn, Wiley & Sons, 1999.
Han et al, Efficient and library-friendly synthesis of furo- and thieno[2,3-d]pyrimidin-4-amine derivatives by microwave irradiation, Tett. Lett., 51, 629-632, 2010.
Jang et al, Synthesis immunosuppressive activity and structure-activity relationship study of a new series of 4-N-piperazinyl-thieno[2,3-d]pyrimidine analogues, Bioorg. Med. Chem. Lett., 20, 844-847, 2010.
Modica et al., Synthesis and binding properties of novel selective 5HT₃ receptor ligands, Bioorg. Med. Chem. Lett., 12, 3891-3901, 2004.
Tasler et al., Thienopyrimidines as β3-adrenoreceptor agonists: Hit to lead optimization, Bioorg. Med. Chem. Lett., 20, 6108-6115, 2010.
Gorja et al., C—C(alkynylation) vs C—O (ether) bond formation under Pd/C—Cu catalysis: synthesis and pharmacological evaluation of 4-alkynylthieno[2,3-d]pyrimidines, Beilstein J. Org. Chem., 7, 338-345, 2011.
(1973). The sick sinus syndrome. Br Med J 2, 677-678.
ALANIS J, GONZALEZ H, & LOPEZ E (1958). The electrical activity of the bundle of His. J Physiol 142, 127-140.
ALANIS J, LOPEZ E, MANDOKI JJ, & Pilar G (1959). Propagation of impulses through the atrioventricular node. Am J Physiol 197, 1171-1174.
Altomare C, Barbuti A, Viscomi C, Baruscotti M, & DiFrancesco D (2000). Effects of dronedarone on Acetylcholine-activated current in rabbit SAN cells. Br J Pharmacol 130, 1315-1320.
Appel, Rolf (1975). Tertiary phosphane/tetrachloroethane, a versatile reagent for chlorination, dehydration and P-N linkage, Angew. Chem. Intl. Ed. Eng., 14 (12), 801-811.
Armstrong C M & Hille B (1998). Voltage-gated ion channels and electrical excitability. Neuron 20, 371-380.
Belardinelli L, Shryock J C, Song Y, Wang D, & Srinivas M (1995). Ionic basis of the electrophysiological actions of adenosine on cardiomyocytes. FASEB J 9, 359-365.
Borchard R, Van B M, Wickenbrock I, Prull M W, Pott L, & Trappe H J. [Inhibition of the muscarinic potassium current by KB130015, a new antiarrhythmic agent to treat atrial fibrillation]. Med Klin. (Munich) 100[11], 697-703. 2005. Ref Type: Abstract
Bosch R F, Zeng X, Grammer J B, Popovic K, Mewis C, & Kuhlkamp V (1999). Ionic mechanisms of electrical remodeling in human atrial fibrillation. Cardiovascular Research 44, 121-131.
Brodde O E & Michel M C (1999). Adrenergic and muscarinic receptors in the human heart. Pharmacol Rev 51, 651-690.
Brundel B J, Van Gelder I C, Henning R, Tieleman R G, Tuinenburg A E, Wietses M, Grndjean J G, Van Gilst W H, & Crijns H J (2001a). Alterations in potassium channel gene expression in atria of patients with persistent and paroxysmal atrial fibrillation: differential regulation of protein and mRNA levels for K+ channels. ACC Current Journal Review 10, 71-72.
Brundel B J, Van G, Henning R H, Tieleman R G, Tuinenburg A E, Wietses M, Grandjean J G, Van G, & Crijns H J (2001b). Ion channel remodeling is related to intraoperative atrial effective refractory periods in patients with paroxysmal and persistent atrial fibrillation. Circulation 103, 684-690.
Brundel BJJM, Ausma J, van Gelder I C, Van Der Want J J L, Van Gilst W H, Crijns HJGM, & Henning R H (2002a). Activation of proteolysis by calpains and structural changes in human paroxysmal and persistent atrial fibrillation. Cardiovascular Research 54, 380-389.
Brundel BJJM, Henning R H, Kampinga H H, van Gelder I C, & Crijns HJGM (2002b). Molecular mechanisms of remodeling in human atrial fibrillation. Cardiovascular Research 54, 315-324.
Burashnikov A & Antzelevitch C (2006). Late-phase 3 EAD. A unique mechanism contributing to initiation of atrial fibrillation. Pacing Clin Electrophysiol 29, 290-295.
Burashnikov A, Sicouri S, Di Diego J M, Belardinelli L, & Antzelevitch C (2010). Synergistic effect of the combination of ranolazine and dronedarone to suppress atrial fibrillation. J Am Coll Cardiol 56, 1216-1224.
Camerino D C, Desaphy J F, Tricarico D, Pierno S, & Liantonio A (2008). Therapeutic approaches to ion channel diseases. Adv Genet 64, 81-145.
Cha T J, Ehrlich J R, Chartier D, Qi X Y, Xiao L, & Nattel S (2006). Kir3-based inward rectifier potassium current: potential role in atrial tachycardia remodeling effects on atrial repolarization and arrhythmias. Circulation 113, 1730-1737.
Chan K W, Langan M N, Sui J L, Kozak J A, Pabon A, Ladias J A, & Logothetis D E (1996). A recombinant inwardly rectifying potassium channel coupled to GTP-binding proteins. The Journal Of General Physiology 107, 381-397.
Chiou C W, Eble J N, & Zipes D P (1997). Efferent vagal innervation of the canine atria and sinus and atrioventricular nodes. The third fat pad. Circulation 95, 2573-2584.
Colatsky T J, Follmer C H, & Starmer C F (1990). Channel specificity in antiarrhythmic drug action. Mechanism of potassium channel block and its role in suppressing and aggravating cardiac arrhythmias. Circulation 82, 2235-2242.
Corey S & CLAPHAM DE (1998). Identification of native atrial G-protein-regulated inwardly rectifying K⁺ (GIRK4) channel homomultimers. J Biol Chem 273, 27499-27504.
Corey S, Krapivinsky G, Krapivinsky L, & CLAPHAM DE (1998). Number and stoichiometry of subunits in the native atrial G-protein-gated K⁺ channel, I_{KACh}. J Biol Chem 273, 5271-5278.
Coumel P (1994). Paroxysmal atrial fibrillation: a disorder of autonomic tone? Eur Heart J 15 SupplA, 9-16.
Coumel P (1996). Autonomic influences in atrial tachyarrhythmias. J Cardiovasc Electrophysiol 7, 999-1007.
Dhar M S & Plummer H K, III (2006). Protein expression of G-protein inwardly rectifying potassium channels (GIRK) in breast cancer cells. BMC Physiol 6, 8.
Dobrev D, Friedrich A, Voigt N, Jost N, Wettwer E, Christ T, Knaut M, & Ravens U (2005). The G protein-gated potassium current I(_{K,ACh}) is constitutively active in patients with chronic atrial fibrillation. Circulation 112, 3697-3706.
Dobrev D, Graf E, Wettwer E, Himmel H M, Hala O, Doerfel C, Christ T, Schuler S, & Ravens U (2001). Molecular basis of downregulation of G-protein-coupled inward rectifying K⁺ current (I_{K,ACh}) in chronic human atrial fibrillation: decrease in GIRK4 mRNA correlates with reduced I_{K,ACh} and muscarinic receptor-mediated shortening of action potentials. Circulation 104, 2551-2557.
Dobrzynski H, Boyett M R, & Anderson R H (2007). New insights into pacemaker activity: promoting understanding of sick sinus syndrome. Circulation 115, 1921-1932.
Drici M D, Diochot S, Terrenoire C, Romey G, & Lazdunski M (2000). The bee venom peptide tertiapin underlines the role of IKACh in acetylcholine-induced atrioventricular blocks. Br J Pharmacol 131, 569-577.
Duprat F, Lesage F, Guillemare E, Fink M, Hugnot J P, Bigay J, Lazdunski M, Romey G, & Barhanin J (1995). Heterologous multimeric assembly is essential for K⁺ channel activity of neuronal and cardiac G-protein-activated inward rectifiers. Biochem Biophys Res Commun 212, 657-663.
Ehrlich J R (2008). Inward rectifier potassium currents as a target for atrial fibrillation therapy. J Cardiovasc Pharmacol 52, 129-135.
Ehrlich J R, Cha T J, Zhang L, Chartier D, Villeneuve L, Hebert T E, & Nattel S (2004). Characterization of a hyperpolarization-activated time-dependent potassium current in canine cardiomyocytes from pulmonary vein myocardial sleeves and left atrium. J Physiol 557, 583-597.
Ehrlich J R, Nattel S, & Hohnloser S H (2007). Novel anti-arrhythmic drugs for atrial fibrillation management. Curr Vasc Pharmacol 5, 185-195.
Ezekowitz M D, Aikens T H, Brown A, & Ellis Z (2010). The evolving field of stroke prevention in patients with atrial fibrillation. Stroke 41, S17-S20.
Ferrer J, Nichols C G, Makhina E N, SALKOFF L, Bernstein J, Gerhard D, Wasson J, Ramanadham S, & Permutt A (1995). Pancreatic Islet Cells Express a Family of Inwardly Rectifying K[IMAGE] Channel Subunits Which Interact to Form G-protein-activated Channels. J Biol Chem 270, 26086-26091.
Ferrer M I (1968). The sick sinus syndrome in atrial disease. JAMA 206, 645-646.
Ford J W, Stevens E B, Treherne J M, Packer J, & Bushfield M (2002). Potassium channels: gene family, therapeutic relevance, high-throughput screening technologies and drug discovery. Prog Drug Res 58, 133-168.
Gaborit N, Le B S, Szuts V, Varro A, Escande D, Nattel S, & Demolombe S (2007a). Regional and Tissue Specific Transcript Signatures of Ion Channel Genes in the Non-diseased Human Heart. J Physiol.
Gaborit N, Le B S, Szuts V, Varro A, Escande D, Nattel S, & Demolombe S (2007b). Regional and tissue specific transcript signatures of ion channel genes in the non-diseased human heart. J Physiol 582, 675-693.
Geibel J P (2005). Role of potassium in acid secretion. World J Gastroenterol 11, 5259-5265.
Gögelein H, Brendel J, Steinmeyer K, Strubing C, Picard N, Rampe D, Kopp K, Busch A E, & Bleich M (2004). Effects of the atrial antiarrhythmic drug AVE0118 on cardiac ion channels. Naunyn Schmiedebergs Arch Pharmacol 370, 183-192.
Gomes J A, Kang P S, Matheson M, Gough W B, Jr., & El-Sherif N (1981). Coexistence of sick sinus rhythm and atrial flutter-fibrillation. Circulation 63, 80-86.
Gregerson K A, Flagg T P, O'Neill T J, Anderson M, Lauring O, Horel J S, & Welling P A (2001). Identification of G protein-coupled, inward rectifier potassium channel gene products from the rat anterior pituitary gland. Endocrinology 142, 2820-2832.
Guillemare E, Marion A, Nisato D, & Gautier P (2000). Inhibitory effects of dronedarone on muscarinic K+ current in guinea pig atrial cells. J Cardiovasc Pharmacol 36, 802-805.
Gutman G A, Chandy K G, Adelman J P, Aiyar J, Bayliss D A, Clapham D E, Covarriubias M, Desir G V, Furuichi K, & Ganetzky et a (2003). International Union of Pharmacology. XLI. Compendium of voltage-gated ion channels: potassium channels. Pharmacological Reviews 55, 583-586.
Haissaguerre M, Jais P, Shah D C, Takahashi A, Hocini M, Quiniou G, Garrigue S, Le M A, Le M P, & Clementy J (1998). Spontaneous initiation of atrial fibrillation by ectopic beats originating in the pulmonary veins. N Engl J Med 339, 659-666.
Han S Y & Bolter C P (2011). The muscarinic-activated potassium channel always participates in vagal slowing of the guinea-pig sinoatrial pacemaker. Auton Neurosci 164, 96-100.
Hara Y & Kizaki K (2002). Antimalarial drugs inhibit the acetylcholine-receptor-operated potassium current in atrial myocytes. Heart Lung Circ 11, 112-116.
Hashimoto N, Yamashita T, Fujikura N, & Tsuruzoe N (2007). NIP-141, a multiple ion channel blocker, terminates aconitine-induced atrial fibrillation and prevents the rapid pacing-induced atrial effective refractory period shortening in dogs. Europace 9, 246-251.
Hashimoto N, Yamashita T, & Tsuruzoe N (2006). Tertiapin, a selective I_{KACh} blocker, terminates atrial fibrillation with selective atrial effective refractory period prolongation. Pharmacol Res 54, 136-141.
Hashimoto N, Yamashita T, & Tsuruzoe N (2008). Characterization of In Vivo and In Vitro Electrophysiological and Antiarrhythmic Effects of a Novel I_{KACh} Blocker, NIP-151: A Comparison With an I_{Kr}-Blocker Dofetilide. J Cardiovasc Pharmacol 51, 162-169.
Hedin K E, Lim N F, & CLAPHAM DE (1996). Cloning of a Xenopus laevis inwardly rectifying K+ channel subunit that permits GIRK1 expression of IKACh currents in oocytes. Neuron 16, 423-429.
Hibino H, Inanobe A, Furutani K, Murakami S, Findlay I, & Kurachi Y (2010). Inwardly rectifying potassium channels: their structure, function, and physiological roles. Physiol Rev 90, 291-366.
Hille B, Armstrong C M, & MacKinnon R (1999). Ion channels: from idea to reality. Nat Med 5, 1105-1109.
Hollopeter G, Jantzen H M, Vincent D, Li G, England L, Ramakrishnan V, Yang R B, Nurden P, Nurden A, Julius D, & Conley P B (2001). Identification of the platelet ADP receptor targeted by antithrombotic drugs. Nature 409, 202-207.
Hong C M, Zheng Q S, Liu X T, Shang F J, Wang H T, & Jiang W R (2009). Effects of autoantibodies against M2 muscarinic acetylcholine receptors on rabbit atria in vivo. Cardiology 112, 180-187.
Horikawa-Tanami T, Hirao K, Furukawa T, & Isobe M (2007). Mechanism of the conversion of a pulmonary vein tachycardia to atrial fibrillation in normal canine hearts: role of autonomic nerve stimulation. J Cardiovasc Electrophysiol 18, 534-541.
Huang J L, Wen Z C, Lee W L, Chang M S, & Chen S A (1998). Changes of autonomic tone before the onset of paroxysmal atrial fibrillation. Int J Cardiol 66, 275-283.
Iwanir S & Reuveny E (2008). Adrenaline-induced hyperpolarization of mouse pancreatic islet cells is mediated by G protein-gated inwardly rectifying potassium (GIRK) channels. Pflugers Arch.
Jayachandran J V, Sih H J, Winkle W, Zipes D P, Hutchins G D, & Olgin J E (2000). Atrial fibrillation produced by prolonged rapid atrial pacing is associated with heterogeneous changes in atrial sympathetic innervation. Circulation 101, 1185-1191.
Jin W, Klem A M, Lewis J H, & Lu Z (1999). Mechanisms of inward-rectifier K+ channel inhibition by tertiapin-Q. Biochemistry 38, 14294-14301.
Jin W & Lu Z (1998). A novel high-affinity inhibitor for inward-rectifier K+ channels. Biochemistry 37, 13291-13299.
Jin W & Lu Z (1999). Synthesis of a Stable Form of Tertiapin: A High-Affinity Inhibitor for Inward-Rectifier K+ Channels. Biochemistry 38, 14286-14293.
Kabell G, Buchanan L V, Gibson J K, & Belardinelli L (1994). Effects of adenosine on atrial refractoriness and arrhythmias. Cardiovasc Res 28, 1385-1389.
Kent K M, Epstein S E, Cooper T, & Jacobowitz D M (1974). Cholinergic innervation of the canine and human ventricular conducting system. Anatomic and electrophysiologic correlations. Circulation 50, 948-955.
Kobayashi T, Hirai H, Iino M, Fuse I, Mitsumura K, Washiyama K, Kasai S, & Ikeda K (2009). Inhibitory effects of the antiepileptic drug ethosuximide on G protein-activated inwardly rectifying K⁺ channels. Neuropharmacology 56, 499-506.
Kobayashi T & Ikeda K (2006). G protein-activated inwardly rectifying potassium channels as potential therapeutic targets. Curr Pharm Des 12, 4513-4523.
Kobayashi T, Washiyama K, & Ikeda K (2003) Inhibition of G protein-activated inwardly rectifying K+ channels by fluoxetine (Prozac). Br J Pharmacol 138, 1119-1128.
Kobayashi T, Washiyama K, & Ikeda K (2004) Inhibition of G protein-activated inwardly rectifying K⁺ channels by various antidepressant drugs. Neuropsychopharmacology 29, 1841-1851.
Kobayashi T, Washiyama K, & Ikeda K (2006) Inhibition of G protein-activated inwardly rectifying K⁺ channels by the antidepressant paroxetine. J Pharmacol Sci 102, 278-287.
Kobayashi T, Washiyama K, & Ikeda K (2010) Inhibition of G-protein-activated inwardly rectifying K+ channels by the selective norepinephrine reuptake inhibitors atomoxetine and reboxetine. Neuropsychopharmacology 35, 1560-1569.
Koo S H, Wakili R, Heo J H, Chartier D, Kim H S, Kim S J, Lee J W, Qi X Y, Nattel S, & Cha T J (2010). Role of constitutively active acetylcholine-mediated potassium current in atrial contractile dysfunction caused by atrial tachycardia remodelling. Europace 12, 1490-1497.
Koumi S, Arentzen C E, Backer C L, & Wasserstrom J A (1994). Alterations in muscarinic K+ channel response to acetylcholine and to G protein-mediated activation in atrial myocytes isolated from failing human hearts. Circulation 90, 2213-2224.
Koumi S & Wasserstrom J A (1994). Acetylcholine-sensitive muscarinic K+ channels in mammalian ventricular myocytes. Am J Physiol 266, H1812-H1821.
Kovoor P, Wickman K, Maguire C T, Pu W, Gehrmann J, Berul C I, & Clapham D E (2001). Evaluation of the role of IKACh in atrial fibrillation using a mouse knockout model. Journal of the American College of Cardiology 37, 2136-2143.
Krapivinsky G, Gordon E A, Wickman K, Velimirovic B, Krapivinsky L, & Clapham D (1995). The G-protein-gated atrial K⁺ channel I_{KACh} is a heteromultimer of two inwardly rectifying K⁺-channel proteins. Nature 374, 135-141.
Kurachi Y, Nakajima T, & Sugimoto T (1987). Quinidine inhibition of the muscarine receptor-activated K+ channel current in atrial cells of guinea pig. Naunyn Schmiedebergs Arch Pharmacol 335, 216-218.
Liu L & Nattel S (1997). Differing sympathetic and vagal effects on atrial fibrillation in dogs: role of refractoriness heterogeneity. The American Journal Of Physiology 273, H805-H816.
Lo L W, Chen Y C, Chen Y J, Wongcharoen W, Lin C I, & Chen S A (2007). Calmodulin kinase II inhibition prevents arrhythmic activity induced by alpha and beta adrenergic agonists in rabbit pulmonary veins. Eur J Pharmacol 571, 197-208.
Lomax A E, Rose R A, & Giles W R (2003). Electrophysiological evidence for a gradient of G protein-gated K+ current in adult mouse atria. Br J Pharmacol 140, 576-584.
Luscher C & Slesinger P A (2010). Emerging roles for G protein-gated inwardly rectifying potassium (GIRK) channels in health and disease. Nat Rev Neurosci 11, 301-315.
Machida T, Hashimoto N, Kuwahara I, Ogino Y, Matsuura J, Yamamoto W, Itano Y, Zamma A, Matsumoto R, Kamon J, Kobayashi T, Ishiwata N, Yamashita T, Ogura T, & Nakaya H (2011). Effects of a highly selective acetylcholine-activated K+ channel blocker on experimental atrial fibrillation. Circ Arrhythm Electrophysiol 4, 94-102.
Makary S, Voigt N, Maguy A, Wakili R, Nishida K, Harada M, Dobrev D, & Nattel S (2011). Differential Protein Kinase C Isoform Regulation and Increased Constitutive Activity of Acetylcholine-Regulated Potassium Channels in Atrial Remodeling. Circ Res.
Marban E (2002). Cardiac channelopathies. Nature 415, 213-218.
Mark M D & Herlitze S (2000). G-protein mediated gating of inward-rectifier K+ channels. Eur J Biochem 267, 5830-5836.
Martin P (1977). The influence of the parasympathetic nervous system on atrioventricular conduction. Circ Res 41, 593-599.
Mathie A & Veale E L (2007). Therapeutic potential of neuronal two-pore domain potassium-channel modulators. Curr Opin Investig Drugs 8, 555-562.
Matsuda T, Ito M, Ishimaru S, Tsuruoka N, Saito T, Iida-Tanaka N, Hashimoto N, Yamashita T, Tsuruzoe N, Tanaka H, & Shigenobu K (2006). Blockade by NIP-142, an Antiarrhythmic Agent, of Carbachol-Induced Atrial Action Potential Shortening and GIRK1/4 Channel. Journal of Pharmacological Sciences 101, 303-310.
Miyauchi M, Kobayashi Y, Miyauchi Y, Abe J, Morita N, Iwasaki Y K, Hayashi M, & Takano T (2004). Parasympathetic blockade promotes recovery from atrial electrical remodeling induced by short-term rapid atrial pacing. Pacing Clin Electrophysiol 27, 33-37.
Nagasawa H, Fujiki A, Fujikura N, Matsuda T, Yamashita T, & Inoue H (2002). Effects of a novel class III antiarrhythmic agent, NIP-142, on canine atrial fibrillation and flutter. Circulation Journal: Official Journal Of The Japanese Circulation Society 66, 185-191.
Novelli G, Predazzi I M, Mango R, Romeo F, Mehta J L, Ezekowitz M D, Aikens T H, Brown A, Ellis Z, Rorsman P, Bokvist K, Ammala C, Arkhammar P, Berggren P O, Larsson O, & Wahlander K (2010). Role of genomics in cardiovascular medicine The evolving field of stroke prevention in patients with atrial fibrillation Activation by adrenaline of a low-conductance G protein-dependent K+ channel in mouse pancreatic B cells. World J Cardiol 2, 428-436.
Ogawa M, Zhou S, Tan A Y, Song J, Gholmieh G, Fishbein M C, Luo H, Siegel R J, Karagueuzian H S, Chen L S, Lin S F, & Chen P S (2007). Left stellate ganglion and vagal nerve activity and cardiac arrhythmias in ambulatory dogs with pacing-induced congestive heart failure. J Am Coll Cardiol 50, 335-343.
Pappone C, Rosanio S, Oreto G, Tocchi M, Gugliotta F, Vicedomini G, Salvati A, Dicandia C, Mazzone P, Santinelli V, Gulletta S, & Chierchia S (2000). Circumferential radiofrequency ablation of pulmonary vein ostia: A new anatomic approach for curing atrial fibrillation. Circulation 102, 2619-2628.
Pappone C, Santinelli V, Manguso F, Vicedomini G, Gugliotta F, Augello G, Mazzone P, Tortoriello V, Landoni G, Zangrillo A, Lang C, Tomita T, Mesas C, Mastella E, & Alfieri O (2004). Pulmonary vein denervation enhances long-term benefit after circumferential ablation for paroxysmal atrial fibrillation. Circulation 109, 327-334.
Patterson E, Lazzara R, Szabo B, Liu H, Tang D, Li Y H, Scherlag B J, & Po S S (2006).
Sodium-calcium exchange initiated by the Ca2+ transient: an arrhythmia trigger within pulmonary veins. J Am Coll Cardiol 47, 1196-1206.
Patterson E, Po S S, Scherlag B J, & Lazzara R (2005). Triggered firing in pulmonary veins initiated by in vitro autonomic nerve stimulation. Heart Rhythm 2, 624-631.
Philipson L H, Kuznetsov A, Toth P T, Murphy J F, Szabo G, Ma G H, & Miller R J (1995). Functional expression of an epitope-tagged G protein-coupled K+ channel (GIRK1). J Biol Chem 270, 14604-14610.
Plummer H K, III, Yu Q, Cakir Y, & Schuller H M (2004). Expression of inwardly rectifying potassium channels (GIRKs) and beta-adrenergic regulation of breast cancer cell lines. BMC Cancer 4, 93.
Po S S, Scherlag B J, Yamanashi W S, Edwards J, Zhou J, Wu R, Geng N, Lazzara R, & Jackman W M (2006). Experimental model for paroxysmal atrial fibrillation arising at the pulmonary vein-atrial junctions. Heart Rhythm 3, 201-208.
Rodriguez-Martinez M, rechiga-Figueroa I A, Moreno-Galindo E G, Navarro-Polanco R A, & Sanchez-Chapula J A (2011). Muscarinic-activated potassium current mediates the negative chronotropic effect of pilocarpine on the rabbit sinoatrial node. Pflugers Arch 462, 235-243.
Rorsman P, Bokvist K, Ammala C, Arkhammar P, Berggren P O, Larsson O, & Wahlander K (1991). Activation by adrenaline of a low-conductance G protein-dependent K+ channel in mouse pancreatic B cells. Nature 349, 77-79.
Sarmast F, Kolli A, Zaitsev A, Parisian K, Dhamoon A S, Guha P K, Warren M, Anumonwo J M, Taffet S M, Berenfeld O, & Jalife J (2003). Cholinergic atrial fibrillation: I(K,ACh) gradients determine unequal left/right atrial frequencies and rotor dynamics. Cardiovasc Res 59, 863-873.
Schauerte P, Scherlag B J, Pitha J, Scherlag M A, Reynolds D, Lazzara R, & Jackman W M (2000). Catheter ablation of cardiac autonomic nerves for prevention of vagal atrial fibrillation. Circulation 102, 2774-2780.
Scherlag B J, Yamanashi W, Patel U, Lazzara R, & Jackman W M (2005). Autonomically induced conversion of pulmonary vein focal firing into atrial fibrillation. J Am Coll Cardiol 45, 1878-1886.
Schotten U, Verheule S, Kirchhof P, & Goette A (2011). Pathophysiological mechanisms of atrial fibrillation: a translational appraisal. Physiol Rev 91, 265-325.
Shankar H, Murugappan S, Kim S, Jin J, Ding Z, Wickman K, & Kunapuli S P (2004). Role of G protein-gated inwardly rectifying potassium channels in P2Y12 receptor-mediated platelet functional responses. Blood 104, 1335-1343.
Sharifov O F, Fedorov V V, Beloshapko G G, Glukhov A V, Yushmanova A V, & Rosenshtraukh L V (2004). Roles of adrenergic and cholinergic stimulation in spontaneous atrial fibrillation in dogs. J Am Coll Cardiol 43, 483-490.
Shieh C C, Coghlan M, Sullivan J P, & Gopalakrishnan M (2000). Potassium channels: molecular defects, diseases, and therapeutic opportunities. Pharmacol Rev 52, 557-594.
Sicouri S, Burashnikov A, Belardinelli L, & Antzelevitch C (2009). Synergistic Electrophysiologic and Antiarrhythmic Effects of the Combination of Ranolazine and Chronic Amiodarone in Canine Atria. Circ Arrhythm Electrophysiol.
Steinberg J S (2004). Atrial fibrillation: an emerging epidemic? Heart 90, 239-240.
Sun H, Xing D, Lloyd J, Hennan J K, & Levesque P C. Abstract 21061: Mild IKr inhibition Significantly Enhances IKur-induced Selective Prolongation of Atrial Refractoriness. Circulation 122, A21061. 2010. Ref Type: Abstract
Takahashi Y, Jais P, Hocini M, Sanders P, Rotter M, Rostock T, Hsu L F, Sacher F, Clementy J, & Haissaguerre M (2006). Shortening of fibrillatory cycle length in the pulmonary vein during vagal excitation. J Am Coll Cardiol 47, 774-780.
Tamargo J, Caballero R, Gomez R, Valenzuela C, & Delpon E (2004). Pharmacology of cardiac potassium channels. Cardiovascular Research 62, 9-33.
Tan A Y, Li H, Wachsmann-Hogiu S, Chen L S, Chen P S, & Fishbein M C (2006). Autonomic innervation and segmental muscular disconnections at the human pulmonary vein-atrial junction: implications for catheter ablation of atrial-pulmonary vein junction. J Am Coll Cardiol 48, 132-143.
Tanaka H & Hashimoto N (2007). A Multiple Ion Channel Blocker, NIP-142, for the Treatment of Atrial Fibrillation. Cardiovasc Drug Rev 25, 342-356.
Tellez J O, Dobrzynski H, Greener I D, Graham G M, Laing E, Honjo H, Hubbard S J, Boyett M R, & Billeter R (2006). Differential expression of ion channel transcripts in atrial muscle and sinoatrial node in rabbit. Circ Res 99, 1384-1393.
Thery C, Gosselin B, Lekieffre J, & Warembourg H (1977). Pathology of sinoatrial node. Correlations with electrocardiographic findings in 111 patients. Am Heart J 93, 735-740.
Voigt N, Maguy A, Yeh Y, Qi X, Ravens U, Dobrev D, & Nattel S (2008). Changes in I_{K,ACh} single-channel activity with atrial tachycardia remodelling in canine atrial cardiomyocytes. Cardiovascular Research 77, 35-43.
Voigt N, Rozmaritsa N, Trausch A, Zimniak T, Christ T, Wettwer E, Matschke K, Dobrev D, & Ravens U (2010a) Inhibition of IK,ACh current may contribute to clinical efficacy of class I and class III antiarrhythmic drugs in patients with atrial fibrillation. Naunyn Schmiedebergs Arch Pharmacol 381, 251-259.
Voigt N, Trausch A, Knaut M, Matschke K, Varro A, Van Wagoner D R, Nattel S, Ravens U, & Dobrev D (2010b). Left-to-Right Atrial Inward-Rectifier Potassium Current Gradients in Patients with Paroxysmal Versus Chronic Atrial Fibrillation. Circ Arrhythm Electrophysiol.
Wagner V, Stadelmeyer E, Riederer M, Regitnig P, Gorischek A, Devaney T, Schmidt K, Tritthart H A, Hirschberg K, Bauernhofer T, & Schreibmayer W (2010). Cloning and characterisation of GIRK1 variants resulting from alternative RNA editing of the KCNJ3 gene transcript in a human breast cancer cell line. J Cell Biochem 110, 598-608.
Watanabe Y, Hara Y, Tamagawa M, & Nakaya H (1996) Inhibitory effect of amiodarone on the muscarinic acetylcholine receptor-operated potassium current in guinea pig atrial cells. J Pharmacol Exp Ther 279, 617-624.
Wettwer E, Hala O, Christ T, Heubach J F, Dobrev D, Knaut M, Varro A, & Ravens U (2004). Role of I_{Kur} in controlling action potential shape and contractility in the human atrium: influence of chronic atrial fibrillation. Circulation 110, 2299-2306.
Wickman K, Karschin C, Karschin A, Picciotto M R, & CLAPHAM DE (2000). Brain localization and behavioral impact of the G-protein-gated K+ channel subunit GIRK4. J Neurosci 20, 5608-5615.
Wickman K, NEMEC J, Gendler S J, & CLAPHAM DE (1998). Abnormal heart rate regulation in GIRK4 knockout mice. Neuron 20, 103-114.
Wongcharoen W, Chen Y C, Chen Y J, Chen S Y, Yeh H I, Lin C I, & Chen S A (2007). Aging increases pulmonary veins arrhythmogenesis and susceptibility to calcium regulation agents. Heart Rhythm 4, 1338-1349.
Woodward R, Stevens E B, & Murrell-Lagnado R D (1997). Molecular determinants for assembly of G-protein-activated inwardly rectifying K+ channels. J Biol Chem 272, 10823-10830.
Workman A J, Kane K A, & Rankin A C (2008). Cellular bases for human atrial fibrillation. Heart Rhythm 5, S1-S6.
Wulff H, Castle N A, & Pardo L A (2009). Voltage-gated potassium channels as therapeutic targets. Nat Rev Drug Discov 8, 982-1001.
Wulff H & Zhorov B S (2008). K+ Channel Modulators for the Treatment of Neurological Disorders and Autoimmune Diseases. Chem Rev 108, 1744-1773.
Wulfsen I, Hauber H P, Schiemann D, Bauer C K, & Schwarz J R (2000). Expression of mRNA for voltage-dependent and inward-rectifying K channels in GH3/B6 cells and rat pituitary. J Neuroendocrinol 12, 263-272.
Yamashita T, Murakawa Y, Sezaki K, Inoue M, Hayami N, Shuzui Y, & Omata M (1997). Circadian variation of paroxysmal atrial fibrillation. Circulation 96, 1537-1541.
Yang D, Xi Y, Ai T, Wu G, Sun J, Razavi M, Delapasse S, Shurail M, Gao L, Mathuria N, Elayda M, & Cheng J (2011). Vagal stimulation promotes atrial electrical remodeling induced by rapid atrial pacing in dogs: evidence of a noncholinergic effect. Pacing Clin Electrophysiol 34, 1092-1099.
Yoshimoto Y, Fukuyama Y, Horio Y, Inanobe A, Gotoh M, & Kurachi Y (1999). Somatostatin induces hyperpolarization in pancreatic islet alpha cells by activating a G protein-gated K+ channel. FEBS Lett 444, 265-269.
Zhang C, Yuan G H, Cheng Z F, Xu M W, Hou L F, & Wei F P (2009). The Single Nucleotide Polymorphisms of Kir3.4 Gene and Their Correlation with Lone Paroxysmal Atrial Fibrillation in Chinese Han Population. Heart Lung Circ.

1. A compound of formula (I) 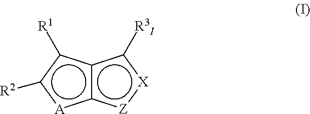
or a pharmaceutically acceptable derivative thereof, wherein:
A is O or S;
X is selected from N, O, CR³_{II} and NR³_{IV};
Z is selected from N, O, CR³_{III} and NR³_{C};
R¹ is selected from H, optionally substituted alkyl, optionally substituted cycloalkyl, optionally substituted aryl, and optionally substituted heteroaryl;
R² is selected from H, halo, —CN, trifluoromethyl, optionally substituted alkyl, optionally substituted alkoxy, —NR⁴R⁵, —NR⁶C(O)R⁷, —NR⁶S(O)₂R⁷, —S(O)₂NR⁴R⁵, —CONR⁴R⁵, —CO₂R⁷, optionally substituted oxazolinyl, —SR¹⁴, —S(O)R¹⁴ and —S(O)₂R¹⁴;
R³_{I} is selected from H, halo, —CN, trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, —C≡C-J, optionally substituted cycloalkyl-J and —(NR^{a}R^{b})-J;
Each of R³_{II} and R³_{III} is independently selected from H, halo, —CN, trifluoromethyl, optionally substituted alkyl, optionally substituted alkoxy, optionally substituted heterocycloalkoxy, optionally substituted heterocycloalkylalkyl, —NR⁶C(O)R⁷, —NR⁶S(O)₂R⁷, —S(O)₂NR⁴R⁵, —CONR⁴R⁵, optionally substituted -alkylene-CONR⁴R⁵, —CO₂R⁷, —SO₂R⁷, —NR¹⁰R¹¹, —C≡C-J, optionally substituted cycloalkyl-J and —(NR^{a}R^{b})-J;
Each of R³_{IV} and R³_{V} is independently selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, —C≡C-J, and optionally substituted cycloalkyl-J;
provided that at least one of R³_{I}, R³_{II} and R³_{III} is present as —C≡C-J, optionally substituted cycloalkyl-J or —(NR^{a}R^{b})-J, or at least one of R³_{IV} and R³_{V} is present as —C≡C-J or optionally substituted cycloalkyl-J;
wherein R^{a} and R^{b} are linked to form an optionally substituted 4 to 7 membered heterocycloalkyl ring, which is optionally bridged by a bond, optionally substituted C₁₋₂alkylene, —NR⁶—, —O—, or —S(O)_{z}—;
J is selected from H and —(CR¹²R¹³)_{q}-L-M-W,
wherein
q is 0, 1 or 2;
L is —O— or —N(G)-; and
G is selected from hydrogen, optionally substituted alkyl, and optionally substituted cycloalkyl;
M is —(CR¹²R¹³)ₜ—;
t is 0, 1, 2 or 3;
W is selected from the group consisting of optionally substituted alkyl, optionally substituted alkoxy, optionally substituted alkenyl, optionally substituted cycloalkyl, optionally substituted heterocycloalkyl, optionally substituted aryl, optionally substituted heteroaryl and —NR⁸R⁹,
wherein when W is optionally substituted cycloalkyl it may optionally be bridged by a bond or optionally substituted C₁₋₂alkylene, and
wherein when W is optionally substituted heterocycloalkyl it may optionally be bridged by a bond, optionally substituted C₁₋₂alkylene, —NR⁶—, —O—, or S(O)_{z}—;
alternatively, when L=—N(G)-, L, G, M and W may be linked to form an optionally substituted heterocycloalkyl, an optionally substituted heterocycloalkenyl, or an optionally substituted heteroaryl;
z is 0, 1 or 2;
R⁴ and R⁵ are, at each instance, independently selected from H, optionally substituted alkyl, optionally substituted aryl, optionally substituted heteroaryl, and optionally substituted cycloalkyl, or are linked to form an optionally substituted heterocycloalkyl;
R⁶ and R⁷ are, at each instance, independently selected from H and optionally substituted alkyl, or are linked to form an optionally substituted heterocycloalkyl;
R⁸ and R⁹ are, at each instance, independently selected from H, optionally substituted alkyl, optionally substituted aryl, optionally substituted heterocycloalkyl, optionally substituted heteroaryl, and optionally substituted cycloalkyl; and
R¹⁰ and R¹¹ are, at each instance, independently selected from H, optionally substituted alkyl, optionally substituted aryl, optionally substituted heterocycloalkyl, optionally substituted heteroaryl, and optionally substituted cycloalkyl;
R¹² and R¹³ are, at each instance, independently selected from H, hydroxy, and optionally substituted alkyl, or may be linked to form an optionally substituted cycloalkyl ring, or may together form ═O; and
R¹⁴ is optionally substituted alkyl,
wherein the optional substitutents are independently selected from halo, trihalomethyl, trihaloethyl, trihalomethoxy, trihaloethoxy, —OH, —NO₂, —CN, —CO₂H, —CO₂C₁₋₆alkyl, —SO₃H, —SOC₁₋₆alkyl, —SO₂C₁₋₆alkyl, —NHSO₂C₁₋₆alkyl, —NC₁₋₆ alkylSO₂C₁₋₆alkyl, —SO₂NH₂, —SO₂NHC₁₋₆alkyl, —SO₂N(C₁₋₆alkyl)₂, —NHSO₂NH₂, —NHSO₂NHC₁₋₆alkyl, —NHSO₂N(C₁₋₆alkyl)₂, —NC₁₋₆alkylSO₂NH₂, —NC₁₋₆alkylSO₂NHC₁₋₆alkyl, —NC₁₋₆alkylSO₂N(C₁₋₆alkyl)₂, —C(═O)H, —C(═O)C₁₋₆alkyl, —NHC(═O)C₁₋₆alkyl, —NC₁₋₆alkylC(═O)C₁₋₆alkyl, C₁₋₆alkylenedioxy, ═O, —N(C₁₋₆alkyl)₂, —C(═O)NH₂, —C(═O)NHC₁₋₆alkyl, —C(═O)N(C₁₋₆alkyl)₂, —NHC(═O)NH₂, —NHC(═O)NHC₁₋₆alkyl, —NHC(═O)N(C₁₋₆alkyl)₂, —NC₁₋₆alkylC(═O)NH₂, —NC₁₋₆alkylC(═O)NHC₁₋₆ alkyl, —NC₁₋₆alkylC(═O)N(C₁₋₆alkyl)₂, —C(═NH)NH₂, —C(═NH)NHC₁₋₆alkyl, —C(═NH)N(C₁₋₆alkyl)₂, —C(═NC₁₋₆alkyl)NH₂, —C(—NC₁₋₆alkyl)NHC₁₋₆alkyl, —C(═NC₁₋₆alkyl)N(C₁₋₆alkyl)₂, —C₁₋₆alkyl, —C₃₋₆cycloalkyl, —C₃₋₆heterocycloalkyl, 2-imidazolidinon-3-yl, 1-C₁₋₆alkyl-2-imidazolidinon-3-yl, C₁₋₆alkylC₃₋₆heterocycloalkyl, aryl, haloaryl, C₁₋₆alkoxyaryl, —C₁₋₆alkylene-NHSO₂C₁₋₆alkyl, —C₁₋₆alkylene-NC₁₋₆alkylSO₂C₁₋₆alkyl, —C₁₋₆alkylene-SO₂NH₂, —C₁₋₆alkylene-SO₂NHC₁₋₆alkyl, —C₁₋₆alkylene-SO₂N(C₁₋₆alkyl)₂, —Z^{t}H, —Z^{t}—C₁₋₆alkyl, —C₁₋₆alkylene-Z^{t}H, —Z^{t}—C₃₋₆cycloalkyl, or —C(═O)NHC₁₋₆alkylene-Z^{t}H wherein Z^{t} is independently O, S, NH or N(C₁₋₆alkyl).

2. The compound of claim 1, wherein A is S, X is N and Z is NR³_{V}.

3. The compound of claim 1, wherein R¹ is phenyl.

4. The compound of claim 1, wherein R² is selected from H, trifluoromethyl, substituted alkyl, optionally substituted alkoxy, —NR⁴R⁵, —NR⁶C(O)R⁷, —S(O)₂NR⁴R⁵, —CONR⁴R⁵, —CO₂R⁷ optionally substituted oxazolinyl, —SR¹⁴, —S(O)R¹⁴ and —S(O)₂R¹⁴.

5. The compound of claim 1, wherein R³_{I} is selected from trifluoromethyl, optionally substituted alkyl, optionally substituted alkoxy, optionally substituted heterocycloalkoxy, —NR⁶C(O)R⁷, —NR⁶S(O)₂R⁷, —S(O)₂NR⁴R⁵, —CONR⁴R⁵, —CO₂R⁷, —NR⁸R⁹, optionally substituted cycloalkyl-J and —(NR^{a}R^{b})-J.

6. The compound of claim 1, wherein R³_{V} is selected from H, —CN, trifluoromethyl, optionally substituted alkyl, optionally substituted heterocycloalkylalkyl, —S(O)₂NR⁴R⁵, —CONR⁴R⁵, optionally substituted -alkylene-CONR⁴R⁵, —CO₂R⁷, —C(O)R⁷, —SO₂R⁷, and optionally substituted cycloalkyl-J.

7. The compound of claim 1, wherein R³_{V} is selected from H, optionally substituted alkyl, —C(O)R⁷, and —SO₂R⁷.

8. The compound of claim 1, wherein R³_{I} is —(NR^{a}R^{b})-J and J is —(CR¹²R¹³)_{q}-L-M-W.

9. The compound of claim 1, wherein q is 0 or 1.

10. The compound of claim 1, wherein q is 1.

11. The compound of claim 1, wherein t is 0, 1 or 2.

12. The compound of claim 1, wherein t is 2.

13. The compound of claim 1, wherein L is O.

14. The compound of claim 1, wherein L is —N(G)-.

15. The compound of claim 1, wherein R¹² and R¹³ are, at each instance, H.

16. The compound of claim 1, wherein W is optionally substituted heterocycloalkyl.

17. A pharmaceutical composition comprising at least one compound as claimed in claim 1 and, optionally, one or more pharmaceutically acceptable excipients.

18. A compound or composition as claimed in claim 1 for use in therapy.

19. A method for the treatment of a disease or condition that is mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, comprising administering to a subject an effective amount of at least one compound or composition as claimed in claim 1.

20. The method of claim 19, wherein the method is for the treatment of cardiovascular diseases, such as atrial fibrillation (AF), atrial flutter (AFL), atrioventricular (AV) dysfunction and sinoatrial node (SAN) dysfunction; the prevention of recurrence of supraventriclar arrhythmias including AF and AFL; the maintenance of sinus rhythm; the termination and cardioversion of supraventriclar arrhythmias; the treatment of sinus node dysfunction; the treatment of AV node dysfunction, including AV block; the treatment of conduction dysfunction; the prevention or reversal of atrial structural and ionic remodeling; the prevention of thrombosis, thromboembolism and thromboembolic diseases, such as stroke, myocardial infarction, and peripheral vascular diseases; the improvement of cardiac contractility; the treatment of metabolic diseases, such as diabetes mellitus; the modulation of neuro-endocrine function; the modulation of the secretion of pituitary hormones; the treatment of neurological and neuropsychiatric disorders, such as pain, depression, anxiety, attention deficit/hyperactivity disorder and epilepsy; and the treatment of cancer, such as breast cancer.

21. A compound or composition as claimed in claim 1 for use in a method for the treatment of a disease or condition that is mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof, comprising administering to a subject an effective amount of at least one compound of formula (I) or composition comprising at least one compound of formula (I).

22. The compound or composition as claimed in claim 21, wherein the method is for the treatment of cardiovascular diseases, such as atrial fibrillation (AF), atrial flutter (AFL), atrioventricular (AV) dysfunction and sinoatrial node (SAN) dysfunction; the prevention of recurrence of supraventriclar arrhythmias including AF and AFL; the maintenance of sinus rhythm; the termination and cardioversion of supraventriclar arrhythmias; the treatment of sinus node dysfunction; the treatment of AV node dysfunction, including AV block; the treatment of conduction dysfunction; the prevention or reversal of atrial structural and ionic remodeling; the prevention of thrombosis, thromboembolism and thromboembolic diseases, such as stroke, myocardial infarction, and peripheral vascular diseases; the improvement of cardiac contractility; the treatment of metabolic diseases, such as diabetes mellitus; the modulation of neuro-endocrine function; the modulation of the secretion of pituitary hormones; the treatment of neurological and neuropsychiatric disorders, such as pain, depression, anxiety, attention deficit/hyperactivity disorder and epilepsy; and the treatment of cancer, such as breast cancer.

23. The use of a compound as claimed in claim 1 for the manufacture of a medicament for the treatment of a disease or condition that is mediated by Kᵢᵣ3.1 and/or Kᵢᵣ3.4 or any heteromultimers thereof, or that requires inhibition of Kᵢᵣ3.1 and/or Kᵢᵣ3.4 or any heteromultimers thereof.

24. The use of claim 23 wherein the medicament is for the treatment of cardiovascular diseases, such as atrial fibrillation (AF), atrial flutter (AFL), atrioventricular (AV) dysfunction and sinoatrial node (SAN) dysfunction; the prevention of recurrence of supraventriclar arrhythmias including AF and AFL; the maintenance of sinus rhythm; the termination and cardioversion of supraventriclar arrhythmias; the treatment of sinus node dysfunction; the treatment of AV node dysfunction, including AV block; the treatment of conduction dysfunction; the prevention or reversal of atrial structural and ionic remodeling; the prevention of thrombosis, thromboembolism and thromboembolic diseases, such as stroke, myocardial infarction, and peripheral vascular diseases; the improvement of cardiac contractility; the treatment of metabolic diseases, such as diabetes mellitus; the modulation of neuro-endocrine function; the modulation of the secretion of pituitary hormones; the treatment of neurological and neuropsychiatric disorders, such as pain, depression, anxiety, attention deficit/hyperactivity disorder and epilepsy; and the treatment of cancer, such as breast cancer.
